(19) United States
(12) Patent Application Publication
(10) Pub. No.: US 2025/0084128 A1
(43) Pub. Date: Mar. 13, 2025

(54) ANTIVIRAL STRUCTURALLY-STAPLED SARS-CoV-2 PEPTIDE-CHOLESTEROL CONJUGATES AND USES THEREOF

(71) Applicant: Dana-Farber Cancer Institute, Inc., Boston, MA (US)

(72) Inventors: Loren D. Walensky, Newton, MA (US); Gregory H. Bird, Pelham, NH (US)

(21) Appl. No.: 18/689,565

(22) PCT Filed: Sep. 8, 2022
(86) PCT No.: PCT/US2022/076114

§ 371 Date: Mar. 6, 2024

Related U.S. Application Data
(60) Provisional application No. 63/241,722, filed on Sep. 8, 2021.

(51) Int. Cl. C07K 14/005; A61K 47/54; A61K 47/60; A61P 31/14

(52) U.S. Cl. CPC C07K 14/005; A61K 47/554; A61K 47/60; A61P 31/14; C12N 2770/20022; C12N 2770/20033

(57) ABSTRACT

Disclosed herein are cross-linked peptides either alone or conjugated to PEG(n)-cholesterol (or thiocholesterol) moieties useful for interfering with and inhibiting or preventing coronavirus infection (e.g., infection by SARS-CoV-2). Also disclosed are methods of treating and/or preventing a coronavirus infection (e.g., COVID-19.

### CROSS-REFERENCE TO RELATED APPLICATION

This application is a U.S. National Stage application of International Application No. PCT/US2022/076114, filed Sep. 8, 2022, which claims the benefit of priority of U.S. Provisional Application No. 63/241,722, filed Sep. 8, 2021, the contents of which are incorporated by reference herein in their entirety.

### SEQUENCE LISTING

This application contains a Sequence Listing that has been submitted electronically as an XML file named “00530-0414US1_SL_ST26.XML.” The XML file, created on Nov. 12, 2024, is 74,438 bytes in size. The material in the XML file is hereby incorporated by reference in its entirety.

### TECHNICAL FIELD

This disclosure relates to structurally-stabilized SARS-CoV-2 antiviral peptides alone or conjugated with a generated PEG(n)-cholesterol or PEG(n)-thiocholesterol derivatization to further optimize activity and methods for making and using such stapled peptide cholesterol conjugates in the prevention and treatment of a coronavirus infection.

### BACKGROUND

No anti-viral therapeutic currently exists to prevent, treat, or fully treat infection by novel coronavirus (nCoV) outbreaks, such as COVID-19 caused by the Wuhan nCoV (also known as 2019-nCoV or SARS-CoV-2). COVID-19 has been declared a high-risk global health emergency by the World Health Organization (WHO) and has, as of September 2022, caused 603,711,760 cases of respiratory disease and 6,484,136 deaths worldwide. Subsequent variants have posed an ongoing public health challenge, including thwarting the efficacy of vaccine, antibody, and molecular therapeutics.

SARS-CoV-2 contains a surface protein that undergoes a conformational change upon engagement with the host cell, resulting in formation of a six-helix bundle that brings the host and viral membranes together. Although peptide-based inhibition of viral fusion processes is mechanistically feasible and clinically effective (e.g., Fuzeon (i.e., enfurvirtide), approved by the FDA in 2003), the biophysical and pharmacologic liabilities of peptides, including loss of bioactive shape and rapid proteolysis in vivo (e.g., 100 mg self-injected twice daily), have limited broader application of this validated approach. Thus, new strategies for the prophylaxis and/or treatment of COVID-19 infection are urgently required to effectively mitigate outbreaks.

### SUMMARY

This application relates to compositions and methods disclosing peptide stabilizing technology (e.g., stapling) that recapitulates and fortifies the structure of bioactive helices, combined with a method for PEG(n)-cholesterol or thiocholesterol derivatization to generate an optimized and targeted prophylactic and therapeutic agent for prevention and/or treatment of coronavirus (e.g., betacoronavirus such as SARS-CoV-2) infection. By inserting “staples” (e.g., all-hydrocarbon staples) into natural peptides, bioactive-helical structure can be restored and remarkable protease resistance can be conferred by burying the otherwise labile amide bonds at the core of the helical structure and/or restraining amide bonds in a manner that precludes their recognition and proteolysis by the body's proteases. Here, hydrocarbon-stapled and PEG(n)-cholesterol or thiocholesterol derivatized peptide inhibitors of coronavirus (e.g., betacoronavirus such as SARS-CoV-2) are disclosed. These structurally-stabilized peptide inhibitors are used to prevent and/or treat coronavirus (e.g., betacoronavirus such as SARS-CoV-2) infection such as COVID-19.

This disclosure features novel SARS-CoV-2 HR2 stapled peptides. In some instances the HR2 stapled peptide is at least 80%, at least 85%, at least 90%, at least 91%, at least 92%, at least 93%, at least 94%, or at least 95% identical to the amino acid sequence of SEQ ID NO: 6. In some instances the HR2 stapled peptide differs from SEQ ID NO: 10 at 1, 2, 3, 4, 5, 6, 7, 8, 9, or 10 amino acid positions. In certain cases, SEQ ID NO:6 is modified to include the staples in any one of Staple Constructs D, G, K, or N (see, FIG. 12). In some instances these HR2 stapled peptides bind recombinant 5-HB of SARS-CoV-2. In one instance, the 5-HB of SARS-CoV-2 comprises or consists of the sequence of SEQ ID NO: 78. In certain instances, substitutions to SEQ ID NO:6 are made to one or more of the residues on the non-interacting face of the helix (see, FIG. 9) or to one or more of the unbound residues of FIG. 24A. In some instances the HR2 stapled peptide is at least 80%, at least 85%, at least 90%, at least 91%, at least 92%, at least 93%, at least 94%, or at least 95% identical to the amino acid sequence of SEQ ID NO: 10, 13, 17, or 20. In some instances the HR2 stapled peptide differs from SEQ ID NO: 10, 13, 17, or 20 at 1, 2, 3, 4, 5, 6, 7, 8, 9, or 10 amino acid positions except that the two non-natural amino acids that form the intermolecular staple in SEQ ID NO: 10, 13, 17, or 20 are not substituted. In some instances these HR2 stapled peptides bind recombinant 5-HB of SARS-CoV-2. In one instance, the 5-HB of SARS-CoV-2 comprises or consists of the sequence of SEQ ID NO: 78. In some cases, the 1, 2, 3, 4, 5, 6, 7, 8, 9, or 10 amino acid positions that are substituted are on the non-interacting face or the boundary between the interacting and non-interacting faces of the alpha helix (see, FIG. 9). In certain instances, substitutions are made to one or more of the unbound residues of FIG. 24A. In certain instances, 1, 2, 3, 4, 5, 6, 7, 8, 9, or 10 amino acids at the N- and/or C-terminal of SEQ ID NO: 10, 13, 17, or 20 can be deleted. In some instances the stapled peptide is 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some cases, one or more (1, 2, 3, 4, 5, 6, 7, 8, 9, 10, 11, 12, or 13) of the N-terminal amino acids upstream of the first stapling amino acid of SEQ ID NO: 10 (i.e., DISGINASVVNIQ (SEQ ID NO: 35) can be deleted. In some cases, one or more (1, 2, 3, 4, 5, 6, 7, 8, 9, 10, 11, 12, 13, 14, 15, or 16) of the C-terminal amino acids downstream of the second stapling amino acid of SEQ ID NO:10 (i.e., VAKNLNESLIDLQELG (SEQ ID NO: 76) can be deleted. In some cases the stapled peptide is at least 80%, at least 85%, at least 90%, at least 91%, at least 92%, at least 93%, at least 94%, or at least 95% identical to the amino acid sequence of SEQ ID NO: 4 and has the two non-natural amino acids that form the intermolecular staple that are found in SEQ ID NO: 10, 13, 17, or 20. In some cases, the 1, 2, 3, 4, 5, 6, 7, 8, 9, or 10 amino acid positions in SEQ ID NO:4 that are substituted are on the interacting face of the alpha helix In some cases, the 1, 2, 3, 4, 5, 6, 7, 8, 9, or 10 amino acid positions in SEQ ID NO:4 that are substituted are on the non-interacting face or the boundary between the interacting and non-interacting faces of the alpha helix (see, FIG. 9) or are one or more of the unbound residues of FIG. 24A. In some instances these HR2 stapled peptides bind recombinant 5-HB of SARS-CoV-2. In one instance, the 5-HB of SARS-CoV-2 comprises or consists of the sequence of SEQ ID NO: 78.

In some cases, the amino acid sequence comprises or consists of the amino acid sequence set forth in SEQ ID NO: 10 (DISGINASVVNIQ8EIDRLNXVAKNLNESLIDLQELGK), wherein 8 is an (R)-α-(7′-octenyl)alanine or (R)-α-(4′-pentenyl)alanine group, and X is a (S)-α-(4′-pentenyl)alanine or (S)-α-(7′-octenyl)alanine group. In some cases, structurally-stabilized polypeptide comprises an amino acid sequence that is at least 94% identical to the sequence set forth in SEQ ID NO: 10 (DISGINASVVNIQ8EIDRLNXVAKNLNESLIDLQELGK), wherein 8 is a (R)-α-(7′-octenyl)alanine group, and X is a (S)-α-(4′-pentenyl)alanine group. In some cases, the structurally-stabilized polypeptide comprises the amino acid sequence set forth in SEQ ID NO: 10 (DISGINASVVNIQ8EIDRLNXVAKNLNESLIDLQELGK) or variant thereof in which there is one amino acid substitution, wherein 8 is a (R)-α-(7′-octenyl)alanine group, and X is a (S)-α-(4′-pentenyl)alanine group. In some cases, the amino acid sequence comprises or consists of the amino acid sequence set forth in SEQ ID NO: 13 (DISGINASVVNIQKEI8RLNEVAXNLNESLIDLQELGK), wherein 8 is an (R)-α-(7′-octenyl)alanine or (R)-α-(4′-pentenyl)alanine group, and X is an (S)-α-(4′-pentenyl)alanine or (S)-α-(7′-octenyl)alanine group. In some cases, the amino acid sequence comprises or consists of the amino acid sequence set forth in SEQ ID NO: 17 (DISGINASVVNIQKEIDRLN8VAKNLNXSLIDLQELGK), wherein 8 is an (R)-α-(7′-octenyl)alanine or (R)-α-(4′-pentenyl)alanine group, and X is an (S)-α-(4′-pentenyl)alanine or (S)-α-(7′-octenyl)alanine group. In some cases, 8 is a (R)-α-(7′-octenyl)alanine group, and X is a (S)-α-(4′-pentenyl)alanine group. In some cases, the amino acid sequence comprises or consists of the amino acid sequence set forth in SEQ ID NO: 17, wherein 8 is an (R)-α-(7′-octenyl)alanine or (R)-α-(4′-pentenyl)alanine group, and X is an (S)-α-(4′-pentenyl)alanine or (S)-α-(7′-octenyl)alanine group. In some cases, 8 is a (R)-α-(7′-octenyl)alanine group, and X is a (S)-α-(4′-pentenyl)alanine group. In some cases, the amino acid sequence comprises or consists of the amino acid sequence set forth in SEQ ID NO: 20, wherein 8 is an (R)-α-(7′-octenyl)alanine or (R)-α-(4′-pentenyl)alanine group, and X is an (S)-α-(4′-pentenyl)alanine or (S)-α-(7′-octenyl)alanine group. In some cases, 8 is a (R)-α-(7′-octenyl)alanine group, and X is a (S)-α-(4′-pentenyl)alanine group.

In some cases, each of the above stapled peptides has either a GK or a K at the C-terminal of the stapled peptide. Each of these stapled peptides can inhibit and/or prevent infection of a cell (e.g., lung epithelial cell) by a SARS-CoV-2 or a variant thereof. In some cases, the stapled peptide inhibits infection of a cell by SARS-CoV-2 in pseudovirus and/or live SARS-CoV-2 virus assays. In some cases, the stapled peptide prevents infection of a cell by SARS-CoV-2 in the pseudovirus and/or the live SARS-CoV-2 virus assays. In some cases, these stapled peptides are conjugated to cholesterol or thiocholesterol. The conjugation may be via a linker such as polyethylene glycol. In some cases, the linker is PEG(n) wherein n=1 to 36. In some cases, n=3-10, 12, 16, 20, 24, or 36. In some cases, n=4 or 8. In some cases, n=4, 5, 6, 7, 8, 10, 12, 14, 16, 18, 20, 22, 24, 26, 28, 30, 32, 34, or 36. In some cases the linker is a peptide linker. In some cases, the linker has a length of or about the same as (PEG)₄ or (PEG)₈. In some cases, the linker/cholesterol has the structure shown as “*” in FIG. 12. These stapled peptides and stapled peptide conjugates can be used to treat or prevent a SARS-CoV-2 infection in a subject in need thereof. In some cases, the subject is a human subject.

The disclosure also features a linker comprising PEG(n)-cholesterol or PEG(n)-thiocholesterol, wherein n=3 to 36. In some cases, n=3-10, 12, 16, 20, 24, or 36. In some cases, n=4, 5, 6, 7, or 8. In some cases, n=4, 5, 6, 7, 8, 10, 12, 14, 16, 18, 20, 22, 24, 26, 28, 30, 32, 34, or 36.

The disclosure also features a stapled HR2 SARS-CoV-2 peptide conjugated to cholesterol or thiocholesterol via a linker (e.g., PEG). In some cases there are 3 to 36 repeats of PEG. The stapled HR2 peptide can be 18 to 60 amino acids in length (e.g., 19 to 50, or 38 to 50 amino acids in length). In some cases the peptide comprises a sequence that is at least 60%, 65%, 70%, 75%, 80%, 85%, 90%, or 95% identical to SEQ ID NO: 3. In some cases the peptide comprises a sequence that is at least 60%, 65%, 70%, 75%, 80%, 85%, 90%, or 95% identical to SEQ ID NO: 4. In some cases, the 1, 2, 3, 4, 5, 6, 7, 8, 9, or 10 amino acid positions that are substituted are on the interacting face of the alpha helix In some cases, the 1, 2, 3, 4, 5, 6, 7, 8, 9, or 10 amino acid positions that are substituted are on the non-interacting face or the boundary between the interacting and non-interacting faces of the alpha helix (see, FIG. 9). In some cases the staple is inserted as the same positions as in SEQ ID NO: 10. In some cases the staple is inserted as the same positions as in SEQ ID NO: 13. In some cases the staple is inserted as the same positions as in SEQ ID NO: 17. In some cases the staple is inserted as the same positions as in SEQ ID NO: 20. In some cases, the stapled peptide inhibits infection of a cell by SARS-CoV-2 in pseudovirus and/or live SARS-CoV-2 virus assays. In some cases, the stapled peptide prevents infection of a cell by SARS-CoV-2 in the pseudovirus and/or the live SARS-CoV-2 virus assays. In some cases, the stapled peptide binds to a polypeptide comprising or consisting of the amino acid sequence of SEQ ID NO: 78.

In a first aspect, the disclosure provides a structurally-stabilized polypeptide comprising an amino acid sequence that is at least 55%, 60%, 65%, 70%, 75%, 80%, 85%, 90%, or 94% identical to sequence set forth in SEQ ID NO: 6 (DISGINASVVNIQKEIDRLNEVAKNLNESLIDLQELGK). In some instances, amino acids at positions of SEQ ID NO:6 selected from (wherein position 1 is the N-terminal Aspartic Acid and position 38 is the C-terminal Lysine of SEQ ID NO: 6): (i) positions 14 and 21, (ii) positions 17 and 24, (iii) positions 20 and 27, (iv) positions 21 and 28, or (v) positions 24 and 31, are replaced by α, α-disubstituted non-natural amino acids with olefinic side chains. In one instance, positions 14 and 21 are replaced by α, α-disubstituted non-natural amino acids with olefinic side chains. In one instance, positions 17 and 24 are replaced by α, α-disubstituted non-natural amino acids with olefinic side chains. In one instance, positions 21 and 28 are replaced by α, α-disubstituted non-natural amino acids with olefinic side chains. In one instance, positions 24 and 31 are replaced by α, α-disubstituted non-natural amino acids with olefinic side chains. In some instances, the structurally-stabilized peptide is 36 to 60 amino acids in length, optionally 38 to 50 amino acids in length (e.g., 36, 37, 38, 39, 40, 41, 42, 43, 44, 45, 46, 47, 48, 49, 50, 51, 52, 53, 54, 55, 56, 57, 58, 59, or 60 amino acids in length. In some instances, the structurally-stabilized peptide is 38 amino acids in length. In some instances, the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally-stabilized peptide binds to a polypeptide comprising or consisting of the amino acid sequence of SEQ ID NO: 78.

In some instances, the amino acid sequence of the structurally-stabilized peptide is at least 70% identical (e.g., at least 70%, 75%, 80%, 85%, 90%, or 94% identical) to the sequence set forth in SEQ ID NO: 6. In some instances, the amino acid sequence of the structurally-stabilized peptide is at least 80% identical (e.g., at least 80%, 85%, 90%, or 94% identical) to the sequence set forth in SEQ ID NO: 6.

In some instances, the amino acid sequence of the structurally-stabilized peptide comprises or consists of the amino acid sequence set forth in SEQ ID NO: 10 (DISGINASVVNIQ8EIDRLNXVAKNLNESLIDLQELGK). In some instances, the amino acid sequence of the structurally-stabilized peptide comprises or consists of the amino acid sequence set forth in SEQ ID NO: 13 (DISGINASVVNIQKEI8RLNEVAXNLNESLIDLQELGK). In some instances, the amino acid sequence of the structurally-stabilized peptide comprises or consists of the amino acid sequence set forth in SEQ ID NO: 17. In some instances, the amino acid sequence of the structurally-stabilized peptide comprises or consists of the amino acid sequence set forth in SEQ ID NO: 20.

In some instances, the structurally-stabilized polypeptide is 38 to 45 (38, 39, 40, 41, 42, 43, 44, or 45) amino acids in length. In some instances, the structurally-stabilized polypeptide is 38 to 40 (38, 39, or 40) amino acids in length.

In some instances, positions 37 and/or 38 of SEQ ID NO: 6 are not substituted.

In a second aspect, the disclosure relates to a structurally stabilized peptide comprising an amino acid sequence set forth in SEQ ID NO: 10, 13, 17, or 20 with at least one to eighteen of the amino acids at positions 3, 4, 6, 9, 11, 13, 15, 18, 20, 22, 25, 27, 29, 32, 34, 35, 37, or 38 in SEQ ID NO: 10 substituted by any other natural or non-natural amino acid wherein the non-natural amino acids at positions 14 and 21, 17 and 24, 21 and 28, or 24 and 31, respectively, of SEQ ID NO: 6 are not substituted, and, wherein the structurally stabilized peptide is no greater than 45 amino acids in length and wherein the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in pseudovirus and/or live SARS-CoV-2 virus assays. In some instances, the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally-stabilized peptide binds to a polypeptide comprising or consisting of the sequence of SEQ ID NO: 78.

In a third aspect, the disclosure features conjugates having any one of the structurally-stabilized polypeptides previously described (e.g., having any one of SEQ ID NO: 7-21). In some instances, the conjugate also includes polyethylene glycol (PEG) and/or a cholesterol moiety (e.g., cholesterol; thiocholesterol). In some instances, the PEG and/or cholesterol are linked to the C-terminus of the structurally-stabilized polypeptide. In some instances, the conjugate comprise PEG and cholesterol. In some instances, the conjugate comprises PEG(n)-cholesterol, wherein n is 1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36), optionally wherein n is 4, 5, 6, 7, or 8. In some cases, n=4, 5, 6, 7, 8, 10, 12, 14, 16, 18, 20, 22, 24, 26, 28, 30, 32, 34, or 36. In some instances, the conjugate comprise PEG and thiocholesterol. In some instances, the conjugate comprises PEG(n)-thiocholesterol, wherein n is 1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36), optionally wherein n is 4, 5, 6, 7, or 8. In some cases, n=4, 5, 6, 7, 8, 10, 12, 14, 16, 18, 20, 22, 24, 26, 28, 30, 32, 34, or 36.

Also featured herein is a conjugate comprising the structurally-stabilized polypeptide of SEQ ID NO: 10 (DISGINASVVNIQ8EIDRLNXVAKNLNESLIDLQELGK) or any one of SEQ ID NOs.: 13, 17, or 20, and a PEG(4)-cholesterol moiety linked to the C-terminal lysine of the structurally-stabilized polypeptide. In some instances, the PEG(4)-cholesterol moiety comprises the formula:

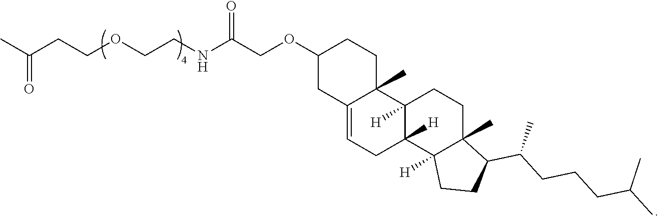

In some instances, the PEG(4)-cholesterol moiety has the formula:

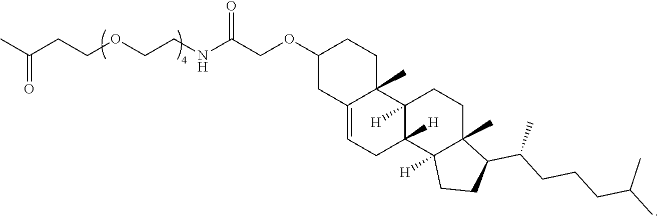

Additionally, featured herein is a conjugate comprising the structurally-stabilized polypeptide of SEQ ID NO: 10 (DISGINASVVNIQ8EIDRLNXVAKNLNESLIDLQELGK), or any one of SEQ ID NO: 13, 17, or 20, and a PEG(4)-thiocholesterol moiety linked to the C-terminal lysine of the structurally-stabilized polypeptide. In some instances, the PEG(4)-thiocholesterol moiety comprises the formula:

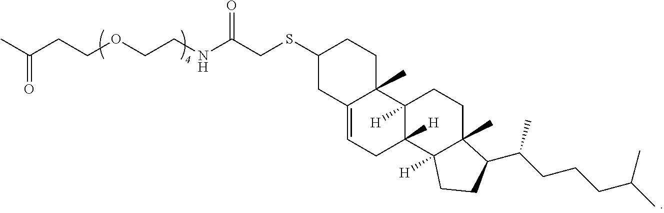

In some instances, the PEG(4)-thiocholesterol moiety has the formula:

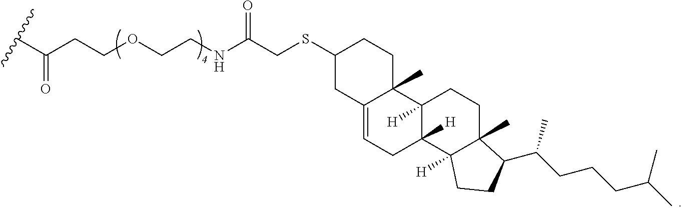

Further featured herein is a conjugate comprising the structurally-stabilized polypeptide of SEQ ID NO: 10 (DISGINASVVNIQ8EIDRLNXVAKNLNESLIDLQELGK), or any one of SEQ ID NO: 13, 17, or 20, and a PEG(8)-cholesterol moiety linked to the C-terminal lysine of the structurally-stabilized polypeptide. In some instances, the PEG(8)-cholesterol moiety comprises the formula:

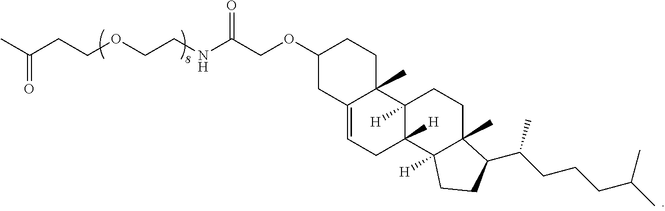

In some instances, the PEG(8)-cholesterol moiety has the formula:

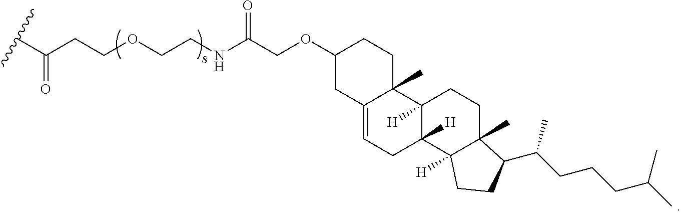

Also featured herein is a conjugate comprising the structurally-stabilized polypeptide of SEQ ID NO: 10 (DISGINASVVNIQ8EIDRLNXVAKNLNESLIDLQELGK), or any one of SEQ ID NO: 13, 17, or 20, and a PEG(8)-thiocholesterol moiety linked to the C-terminal lysine of the structurally-stabilized polypeptide. In some instances, the PEG(8)-thiocholesterol moiety comprises the formula:

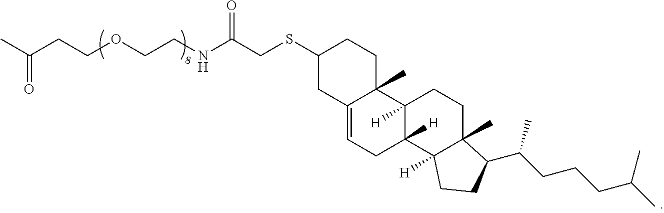

In some instances, the PEG(8)-thiocholesterol moiety has the formula:

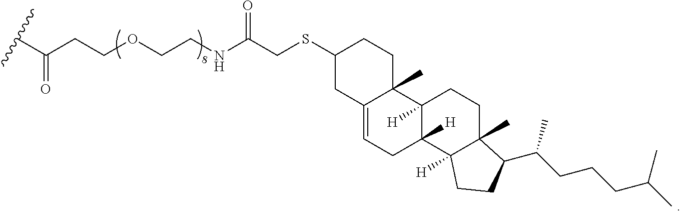

Additionally featured herein is a conjugate comprising the structurally-stabilized polypeptide of SEQ ID NO: 10 (DISGINASVVNIQ8EIDRLNXVAKNLNESLIDLQELGK), or any one of SEQ ID NO: 13, 17, or 20, and a PEG(n)-cholesterol moiety linked to the C-terminal lysine of the structurally-stabilized polypeptide, wherein (n)=4, 5, 6, 7, or 8. In some cases, n=4, 5, 6, 7, 8, 10, 12, 14, 16, 18, 20, 22, 24, 26, 28, 30, 32, 34, or 36. In some instances, the PEG(n)-cholesterol moiety comprises the formula:

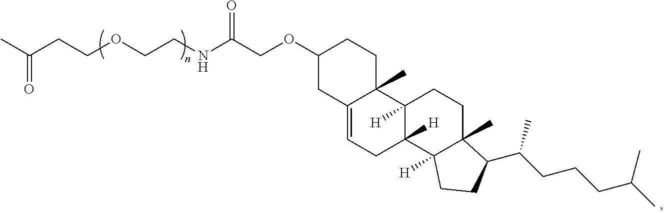

wherein n=4, 5, 6, 7, or 8. In some cases, n=4, 5, 6, 7, 8, 10, 12, 14, 16, 18, 20, 22, 24, 26, 28, 30, 32, 34, or 36.
   In some instances, the PEG(n)-cholesterol moiety has the formula:

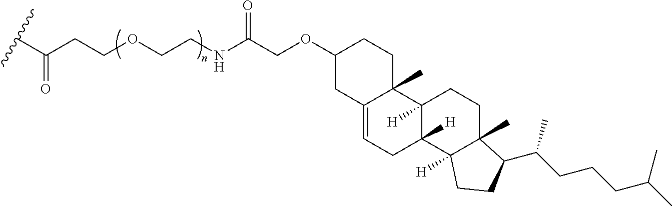

wherein n=4, 5, 6, 7, or 8. In some cases, n=4, 5, 6, 7, 8, 10, 12, 14, 16, 18, 20, 22, 24, 26, 28, 30, 32, 34, or 36.

Finally, also featured herein is a conjugate comprising the structurally-stabilized polypeptide of SEQ ID NO: 10 (DISGINASVVNIQ8EIDRLNXVAKNLNESLIDLQELGK), or any one of SEQ ID NO: 13, 17, or 20, and a PEG(n)-thiocholesterol moiety linked to the C-terminal lysine of the structurally-stabilized polypeptide, wherein (n)=4, 5, 6, 7, or 8. In some cases, n=4, 5, 6, 7, 8, 10, 12, 14, 16, 18, 20, 22, 24, 26, 28, 30, 32, 34, or 36. In some instances, the PEG(n)-thiocholesterol moiety comprises the formula:

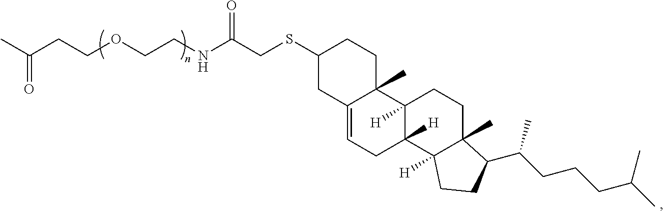

wherein n=4, 5, 6, 7, or 8. In some cases, n=4, 5, 6, 7, 8, 10, 12, 14, 16, 18, 20, 22, 24, 26, 28, 30, 32, 34, or 36.

In some instances, the PEG(n)-thiocholesterol moiety has the formula:

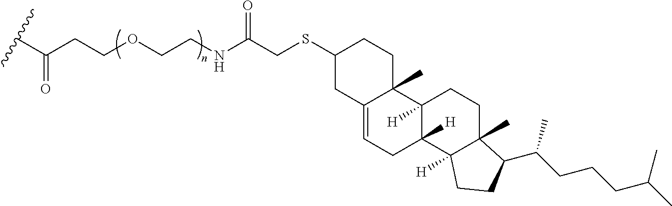

wherein n=4, 5, 6, 7, or 8. In some cases, n=4, 5, 6, 7, 8, 10, 12, 14, 16, 18, 20, 22, 24, 26, 28, 30, 32, 34, or 36.

Also featured herein is a conjugate comprising: a structurally-stabilized polypeptide having an amino acid sequence that is at least 94% identical to SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(4)-cholesterol moiety linked to the C-terminal lysine of the structurally-stabilized polypeptide.

In some instances, the conjugate comprises: the structurally-stabilized polypeptide of SEQ ID NO: 10 (DISGINASVVNIQ8EIDRLNXVAKNLNESLIDLQELGK), or any one of SEQ ID NO: 13, 17, or 20, wherein 8 is a (R)-α-(7′-octenyl)alanine group, and X is a (S)-α-(4′-pentenyl)alanine group; and a PEG(4)-cholesterol moiety linked to the C-terminal lysine of the structurally-stabilized polypeptide.

In some instances, the PEG(4)-cholesterol moiety comprises the formula:

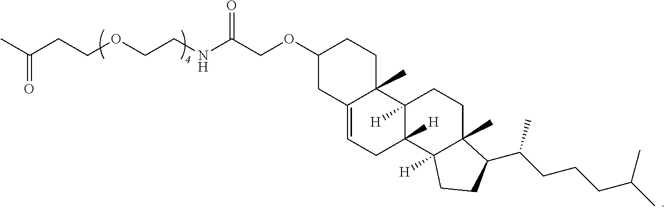

In some instances, the PEG(4)-cholesterol moiety has the formula:

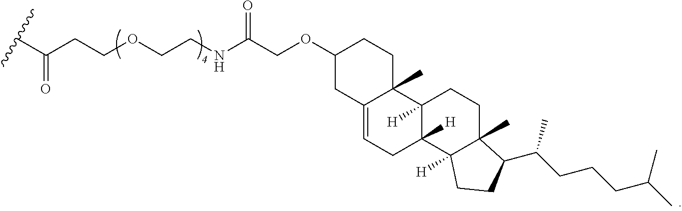

Also featured herein is a conjugate comprising: a structurally-stabilized polypeptide having an amino acid sequence that is at least 94% identical to SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(4)-thiocholesterol moiety linked to the C-terminal lysine of the structurally-stabilized polypeptide.

In some instances, the conjugate comprises: the structurally-stabilized polypeptide of SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(4)-thiocholesterol moiety linked to the C-terminal lysine of the structurally-stabilized polypeptide.

In some instances, the PEG(4)-thiocholesterol moiety comprises the formula:

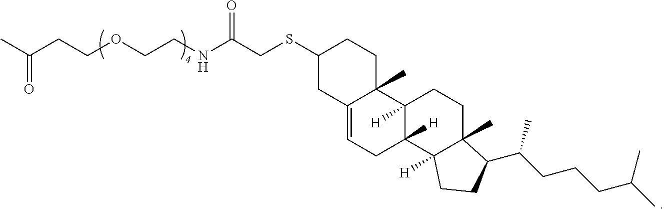

In some instances, the PEG(4)-thiocholesterol moiety has the formula:

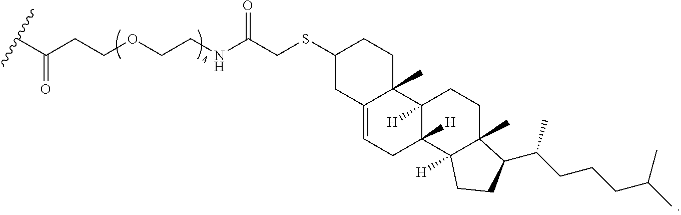

Also featured herein is a conjugate comprising: a structurally-stabilized polypeptide having an amino acid sequence that is at least 94% identical to SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(8)-cholesterol moiety linked to the C-terminal lysine of the structurally-stabilized polypeptide.

In some instances, the conjugate comprises: the structurally-stabilized polypeptide of SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(8)-cholesterol moiety linked to the C-terminal lysine of the structurally-stabilized polypeptide. In some instances, the PEG(8)-cholesterol moiety comprises the formula:

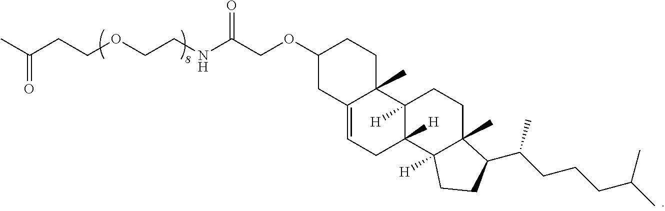

In some instances, the PEG(8)-cholesterol moiety has the formula:

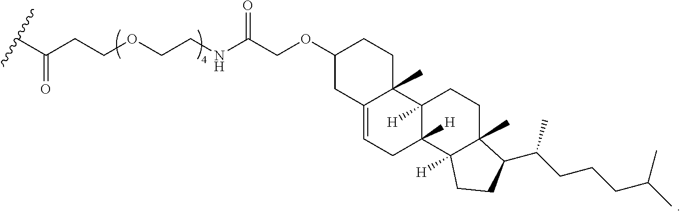

Also featured herein is a conjugate comprising: a structurally-stabilized polypeptide having an amino acid sequence that is at least 94% identical to SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(8)-thiocholesterol moiety linked to the C-terminal lysine of the structurally-stabilized polypeptide. In some instances, the conjugate comprises: the structurally-stabilized polypeptide of SEQ ID NO: 10 (DISGINASVVNIQ8EIDRLNXVAKNLNESLIDLQELGK), or any one of SEQ ID NO:13, 17, or 20, wherein 8 is a (R)-α-(7′-octenyl)alanine group, and X is a (S)-α-(4′-pentenyl)alanine group; and a PEG(8)-thiocholesterol moiety linked to the C-terminal lysine of the structurally-stabilized polypeptide.

In some instances, the PEG(8)-thiocholesterol moiety comprises the formula:

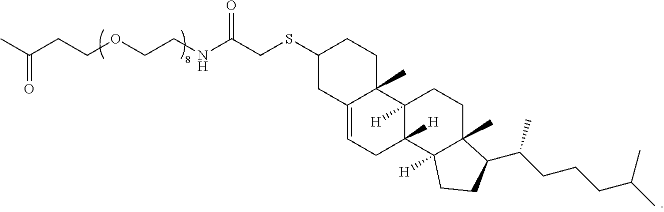

In some instances, the PEG(8)-thiocholesterol moiety has the formula:

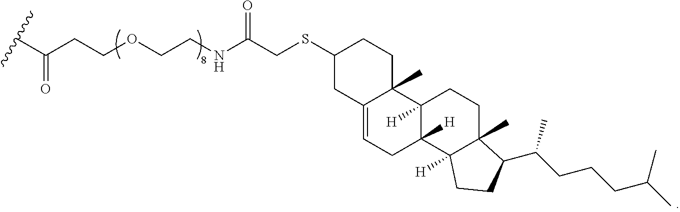

Also featured herein is a conjugate comprising: the structurally-stabilized polypeptide of SEQ ID NO: 10 (DISGINASVVNIQ8EIDRLNXVAKNLNESLIDLQELGK), or any one of SEQ ID NO:13, 17, or 20, wherein 8 is an (R)-α-(7′-octenyl)alanine or (R)-α-(4′-pentenyl)alanine group, and X is a (S)-α-(4′-pentenyl)alanine or (S)-α-(7′-octenyl)alanine group; and a PEG(n)-cholesterol moiety linked to the C-terminal lysine of the structurally-stabilized polypeptide, wherein (n)=4, 5, 6, 7, or 8. In some cases, n=4, 5, 6, 7, 8, 10, 12, 14, 16, 18, 20, 22, 24, 26, 28, 30, 32, 34, or 36. In some instances, the PEG(n)-cholesterol moiety has the formula:

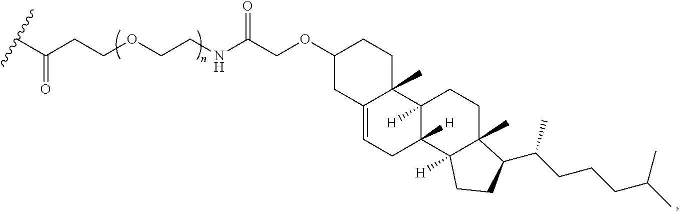

wherein n=4, 5, 6, 7, or 8. In some cases, n=4, 5, 6, 7, 8, 10, 12, 14, 16, 18, 20, 22, 24, 26, 28, 30, 32, 34, or 36.

Also featured herein is a conjugate comprising: the structurally-stabilized polypeptide of SEQ ID NO: 10 (DISGINASVVNIQ8EIDRLNXVAKNLNESLIDLQELGK), or any one of SEQ ID NO:13, 17, or 20, wherein 8 is an (R)-α-(7′-octenyl)alanine or (R)-α-(4′-pentenyl)alanine group, and X is a (S)-α-(4′-pentenyl)alanine or (S)-α-(7′-octenyl)alanine group; and a PEG(n)-thiocholesterol moiety linked to the C-terminal lysine of the structurally-stabilized polypeptide, wherein (n)=4, 5, 6, 7, or 8. In some cases, n=4, 5, 6, 7, 8, 10, 12, 14, 16, 18, 20, 22, 24, 26, 28, 30, 32, 34, or 36. In some instances, the PEG(n)-thiocholesterol moiety has the formula:

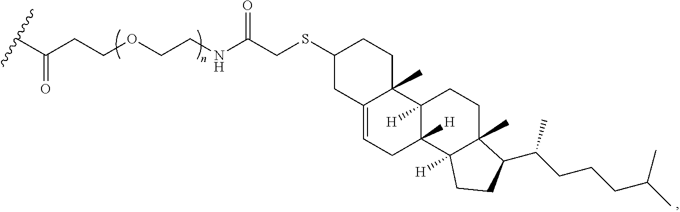

wherein n=4, 5, 6, 7, or 8. In some cases, n=4, 5, 6, 7, 8, 10, 12, 14, 16, 18, 20, 22, 24, 26, 28, 30, 32, 34, or 36.

In some instances, 8 is a (R)-α-(7′-octenyl)alanine group, and X is a (S)-α-(4′-pentenyl)alanine group.

Next, featured herein are structurally-stabilized peptides comprising the formula:

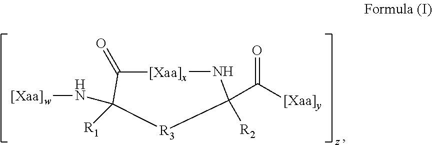

or a pharmaceutically acceptable salt thereof, wherein each R₁ and R₂ is H or a C1 to C10 alkyl, alkenyl, alkynyl, arylalkyl, cycloalkylalkyl, heteroarylalkyl, or heterocyclylalkyl, any of which is substituted or unsubstituted; each R₃ is independently alkane alkylene, alkenylene, or alkynylene, any of which is substituted or unsubstituted; z is 1, 2, 3, 4, 5, 6, 7, 8, 9, or 10; each [Xaa]_{w} is DISGINASVVNIQ (SEQ ID NO: 35), each [Xaa]ₓ is EIDRLN (SEQ ID NO: 36), and each [Xaa]_{y} is VAKNLNESLIDLQELGK (SEQ ID NO: 37); and wherein the structurally-stabilized peptide inhibits infection of a cell by SARS-CoV-2 in in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

In some instances, R₁ is an alkyl. In some instances, R₁ is a methyl group. In some instances, R₃ is an alkyl. In some instances, R₃ is a methyl group. In some instances, R₂ is an alkenyl. In some instances, R₂ is an alkyl. In some instances, R₂ is a methyl group. In some instances, R₃ is an alkenyl. In some instances, the pharmaceutically acceptable salt comprises hydrochloride, sodium, sulfate, acetate, phosphate or diphosphate, chloride, potassium, maleate, calcium, citrate, mesylate, nitrate, tartrate, aluminum, gluconate, and any combination thereof.

In some instances, the structurally-stabilized peptide or pharmaceutically acceptable salt described herein is at most 50 (e.g., 38, 39, 40, 41, 42, 43, 44, 45, 46, 47, 48, 49, or 50) amino acids in length, optionally at most 45 (e.g., 45, 46, 47, 48, 49, or 50) amino acids in length. In some instances, the structurally-stabilized peptide or pharmaceutically acceptable salt thereof is 38 amino acids in length.

Also featured herein is a structurally stabilized peptide comprising Formula I-1, wherein Formula I-1 is defined by:

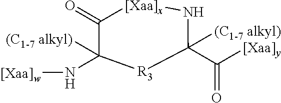

or a pharmaceutically acceptable salt thereof;
R₃ is alkenylene;

[Xaa]_{w} is
(SEQ ID NO: 35)
DISGINASVVNIQ;
[Xaa]ₓ is
(SEQ ID NO: 36)
EIDRLN;
and
[Xaa]_{y} is
(SEQ ID NO: 37)
VAKNLNESLIDLQELGK;

wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In some instances, the structurally stabilized peptide consists of Formula I-1a. In some instances, the structurally stabilized peptide comprises Formula I-la, which is defined by

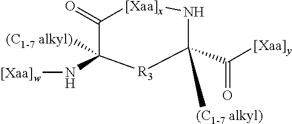

or a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula I-A.

Also featured herein is a structurally stabilized peptide comprising Formula I-A, wherein Formula I-A is defined by:

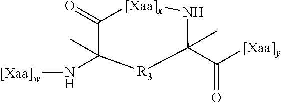

or a pharmaceutically acceptable salt thereof; R₃ is alkenylene;

[Xaa]_{w} is
(SEQ ID NO: 35)
DISGINASVVNIQ;
[Xaa]ₓ is
(SEQ ID NO: 36)
EIDRLN;
and
[Xaa]_{y} is
(SEQ ID NO: 37)
VAKNLNESLIDLQELGK;

wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In some instances, the structurally stabilized peptide consists of Formula I-A.

In some instances, the structurally stabilized peptide comprises Formula Ia, which is defined by

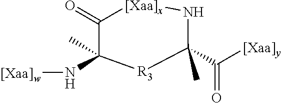

or a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula I-A.

In some instances, the structurally stabilized peptide consists of Formula Ia.

In some instances, the structurally stabilized peptide comprises

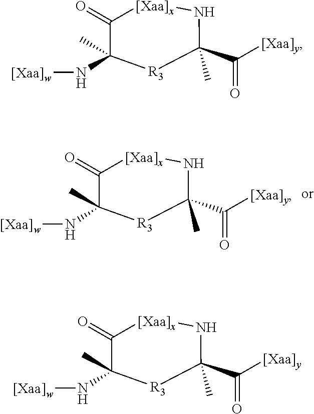

O or a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula I-A.

In some instance, the structurally stabilized peptide consists of

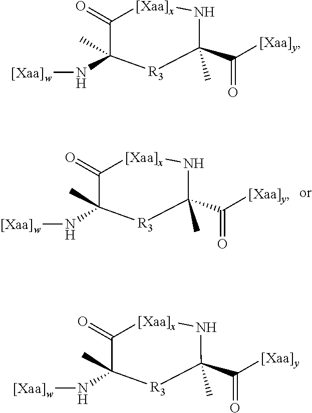

a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula I-A.

In some instances, R₃ is C₇₋₁₅ alkenylene. In some instances, R₃ is C₉₋₁₃ alkenylene. In some instances, R₃ is C₁₁ alkenylen In some instances, R₃ is —(CH₂)₃₋₇—CH═CH—(CH₂)₃₋₇—. In some instances, R₃ is —(CH₂)₅₋₇—CH═CH—(CH₂)₃₋₄—. In some instances, R₃ is —(CH₂)₆—CH═CH—(CH₂)₃—. In some instances, the amino group of the N-terminal aspartic acid in [Xaa]_{w} is replaced with —N(H)C(O)—(C₁₋₄ alkyl). In some instances, the amino group of the N-terminal aspartic acid in [Xaa]_{w} is replaced with —N(H)C(O)CH₃. In some instances, the carboxylic acid group of the C-terminal lysine in [Xaa]_{y} is replaced with —C(O)NH₂

Another feature of the disclosure is a conjugate comprising the structurally-stabilized peptide or pharmaceutically acceptable salt thereof having Formula (I) and PEG and/or cholesterol. In some instances, the conjugate comprises PEG and cholesterol. In some instances, the cholesterol is thiocholesterol. In some instances, the conjugate comprises conjugate comprises PEG(n)-cholesterol, optionally wherein n is 1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36), optionally wherein n is 4, 5, 6, 7, or 8. In some cases, n=4, 5, 6, 7, 8, 10, 12, 14, 16, 18, 20, 22, 24, 26, 28, 30, 32, 34, or 36. In some instances, the conjugate comprises conjugate comprises PEG(n)-thiocholesterol, wherein n is 1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36), optionally wherein n is 4, 5, 6, 7, or 8. In some cases, n=4, 5, 6, 7, 8, 10, 12, 14, 16, 18, 20, 22, 24, 26, 28, 30, 32, 34, or 36.

Also feature herein is a structurally stabilized peptide conjugate comprising Formula II, wherein Formula II is defined by:

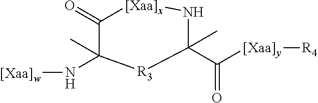

or a pharmaceutically acceptable salt thereof, wherein:
R₃ is alkenylene;
R₄ is **—C(O)—(C₂₋₆ alkylene)-[O—CH₂CH₂]ₘ—N(R₅)C(O)—(C₁₋₆ alkylene)-R₆, wherein ** is the point of attachment to the amino group in the side chain of the C-terminal lysine in [Xaa]_{y};
R₅ is hydrogen or C₁₋₄ alkyl;
R₆ is one of the following:

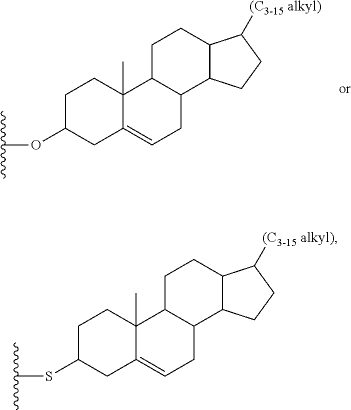

optionally wherein each of which is substituted by t occurrences of R₇;
R₇ represents independently for each occurrence C₁₋₃ alkyl, hydroxyl, or C₁₋₃ alkoxyl:

wherein
[Xaa]_{w} is
(SEQ ID NO: 35)
DISGINASVVNIQ;
[Xaa]ₓ is
(SEQ ID NO: 36)
EIDRLN;
[Xaa]_{y} is
(SEQ ID NO: 37)
VAKNLNESLIDLQELGK;

m is 3, 4, 5, 6, 7, 8, 9, 10, 11, 12, 13, 14, 15, or 16; and
t is 0, 1, 2, or 3;
wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In some embodiments, the structurally stabilized peptide consists of Formula II. In some embodiments, the structurally stabilized peptide comprises Formula IIa, which is defined by:

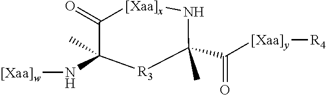

or a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula II. In some embodiments, the structurally stabilized peptide consists of Formula IIa. In some embodiments, the structurally stabilized peptide comprises:

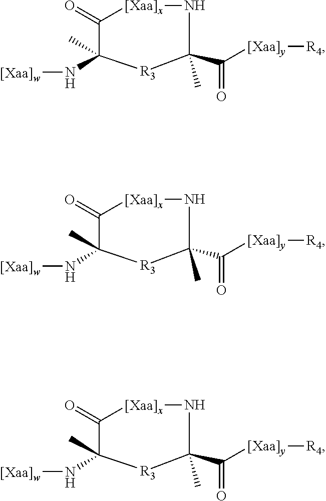

or a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula II.

In some embodiments, the structurally stabilized peptide consists of

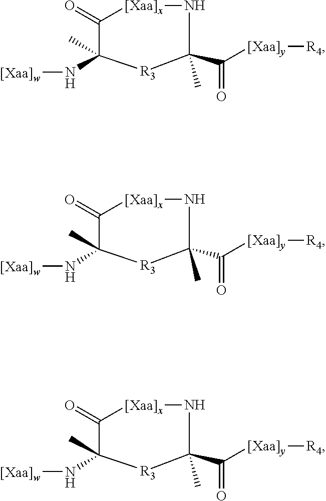

or a pharmaceutically acceptable salt thereof, wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula II.

In some embodiments, R₃ is C₇₋₁₅ alkenylene. In some embodiments, R₃ is C₉₋₁₃ alkenylene. In some embodiments, R₃ is C₁₁ alkenylene. In some embodiments, R₃ is —(CH₂)₃₋₇—CH═CH—(CH₂)₃₋₇—. In some embodiments, R₃ is —(CH₂)₅₋₇—CH═CH—(CH₂)₃₋₄—. In some embodiments, R₃ is —(CH₂)₆—CH═CH—(CH₂)₃—.

Also featured herein is a structurally stabilized peptide conjugate comprising Formula III, wherein Formula III is defined by:

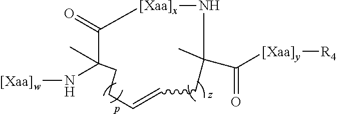

or a pharmaceutically acceptable salt thereof, wherein:
R₄ is **—C(O)—(C₂₋₆ alkylene)-[O—CH₂CH₂]ₘ—N(R₅)C(O)—(C₁₋₆ alkylene)-R₆,
wherein ** is the point of attachment to the amino group in the side chain of the C-terminal lysine in [Xaa]_{y};
R₅ is hydrogen or C₁₋₄ alkyl;
R₆ is one of the following:

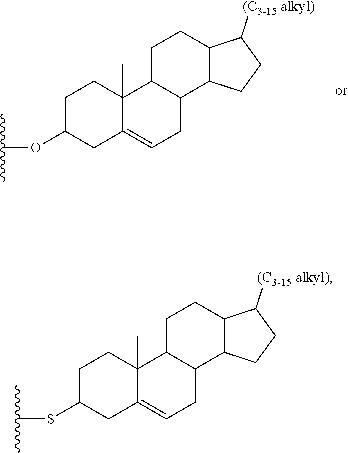

optionally each of which is substituted by t occurrences of R₇;
R₇ represents independently for each occurrence C₁₋₃ alkyl, hydroxyl, or C₁₋₃ alkoxyl;

wherein
[Xaa]_{w} is
(SEQ ID NO: 35)
DISGINASVVNIQ;
[Xaa]ₓ is
(SEQ ID NO: 36)
EIDRLN;
[Xaa]_{y} is
(SEQ ID NO: 37)
VAKNLNESLIDLQELGK;

m is 3, 4, 5, 6, 7, 8, 9, 10, 11, 12, 13, 14, 15, or 16;
p is 2, 3, 4, 5, 6, 7, or 8;
z is 2, 3, 4, 5, or 6; and
t is 0, 1, 2, or 3;
wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In some instances, the structurally stabilized peptide consists of Formula III.

In some instances, the structurally stabilized peptide comprises Formula IIIa, which is defined by:

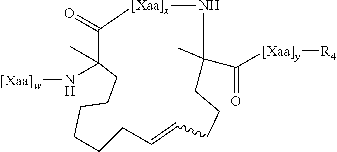

or a pharmaceutically acceptable salt thereof, wherein optionally the amino group of the N-terminal aspartic acid in [Xaa]W is replaced with —N(H)C(O)—(C₁₋₄ alkyl), and optionally the carboxylic acid group of the C-terminal lysine in [Xaa]_{y} is replaced with —C(O)NH₂, wherein the variables are as set forth for Formula III.

In some instances, R₄ is **—C(O)—(C₂₋₃ alkylene)-[O—CH₂CH₂]ₘ—N(R₅)C(O)—(C₁₋₂ alkylene)-R₆, wherein ** is the point of attachment to the amino group in the side chain of the C-terminal lysine in [Xaa]_{y}. In some instances, R₄ is **—C(O)—(CH₂CH₂)—[O—CH₂CH₂]ₘ—N(R₅)C(O)—(CH₂)—R₆, wherein ** is the point of attachment to the amino group in the side chain of the C-terminal lysine in [Xaa]_{y}. In some instances, the amino group of the N-terminal aspartic acid in [Xaa]_{w} is replaced with —N(H)C(O)—(C₁₋₄ alkyl). In some instances, the amino group of the N-terminal aspartic acid in [Xaa]_{w} is replaced with —N(H)C(O)CH₃. In some instances, the carboxylic acid group of the C-terminal lysine in [Xaa]_{y} is replaced with —C(O)NH₂.

Also featured herein is a structurally stabilized peptide conjugate represented by Formula IV:

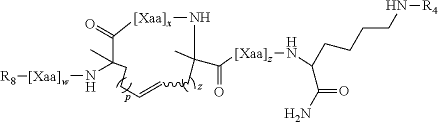

or a pharmaceutically acceptable salt thereof, wherein:
R₄ is —C(O)—(C₂₋₆ alkylene)-[O—CH₂CH₂]ₘ—N(R₅)C(O)—(C₁₋₆ alkylene)-R₆;
R₅ is hydrogen or C₁₋₄ alkyl;
R⁶ is one of the following:

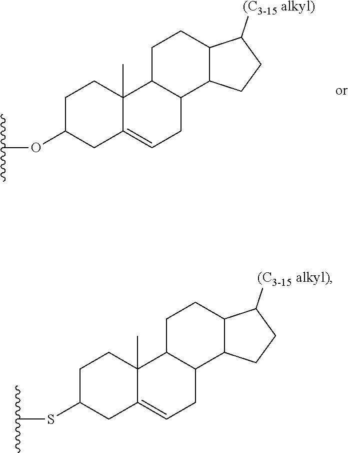

optionally wherein each of which is substituted by t occurrences of R₇;
R₇ represents independently for each occurrence C₁₋₃ alkyl, hydroxyl, or C₁₋₃ alkoxyl;
   wherein R₈ is —C(O)—(C₁₋₄ alkyl);

[Xaa]_{w} is
(SEQ ID NO: 35)
DISGINASVVNIQ;
[Xaa]ₓ is
(SEQ ID NO: 36)
EIDRLN;
[Xaa]_{z} is
(SEQ ID NO: 77)
VAKNLNESLIDLQELG;

m is 3, 4, 5, 6, 7, 8, 9, 10, 11, 12, 13, 14, 15, or 16;
p is 2, 3, 4, 5, 6, 7, or 8;
z is 2, 3, 4, 5, or 6; and
t is 0, 1, 2, or 3.

In some instances, the structurally stabilized peptide is represented by Formula IVa or a pharmaceutically acceptable salt thereof, where Formula IVa is defined by:

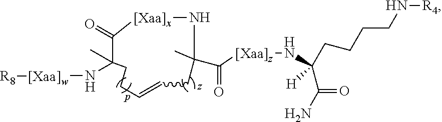

wherein the variables are as set forth for Formula IV.

In some instances, the structurally stabilized peptide is represented by Formula IVb or a pharmaceutically acceptable salt thereof, where Formula IVb is defined by:

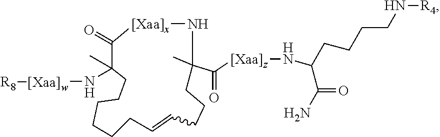

wherein the variables are as set forth for Formula IV.

In some instances, the structurally stabilized peptide is represented by Formula IVc or a pharmaceutically acceptable salt thereof, where Formula IVc is defined by:

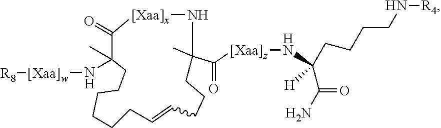

wherein the variables are as set forth for Formula IV.

Also featured herein is a structurally stabilized peptide conjugate represented by Formula V or a pharmaceutically acceptable salt thereof, wherein Formula V is defined by:

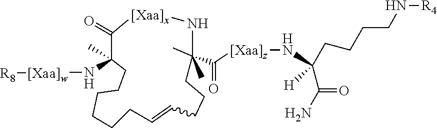

wherein
R₄ is —C(O)—(C₂₋₆ alkylene)-[O—CH₂CH₂]ₘ—N(R₅)C(O)—(C₁₋₆ alkylene)-R₆;
R₅ is hydrogen or C₁₋₄ alkyl;
R⁶ is one of the following:

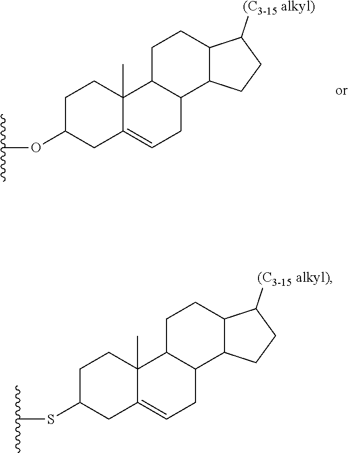

optionally wherein each of which is substituted by t occurrences of R₇;
R₇ represents independently for each occurrence C₁₋₃ alkyl, hydroxyl, or C₁₋₃ alkoxyl;
   wherein R₈ is —C(O)—(C₁₋₄ alkyl);

[Xaa]_{w} is
(SEQ ID NO: 35)
DISGINASVVNIQ;
[Xaa]ₓ is
(SEQ ID NO: 36)
EIDRLN;
[Xaa]_{z} is
(SEQ ID NO: 77)
VAKNLNESLIDLQELG;

m is 3, 4, 5, 6, 7, 8, 9, 10, 11, 12, 13, 14, 15, or 16; and
t is 0, 1, 2, or 3.

In some instances, R₈ is —C(O)CH₃. In some instances, R₄ is —C(O)—(C₂₋₃ alkylene)-[O—CH₂CH₂]ₘ—N(R₅)C(O)—(C₁₋₂ alkylene)-R₆. In some instances, R₄ is —C(O)—(CH₂CH₂)—[O—CH₂CH₂]ₘ—N(R₅)C(O)—(CH₂)—R₆. In some instances, R₅ is hydrogen.

In some instances, R₆ is

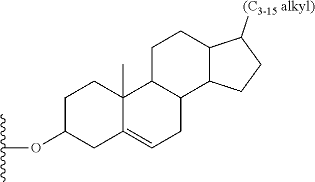

substituted by t occurrences of R₇.

In some instances, R₆ is

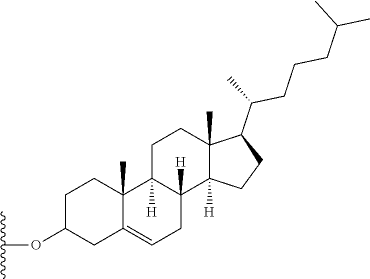

substituted by t occurrences of R₇.

In some instances, R₆ is

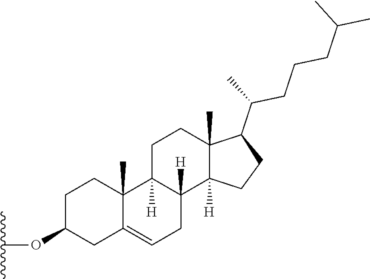

substituted by t occurrences of R₇.

In some instances, R₆ is

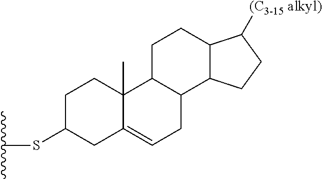

substituted by t occurrences of R₇.

In some instances, R₆ is

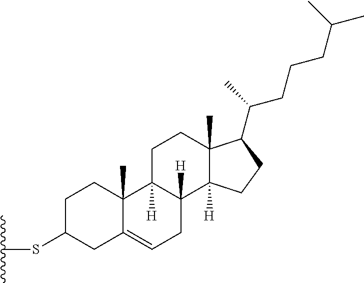

substituted by t occurrences of R₇.

In some instances, R₆ is

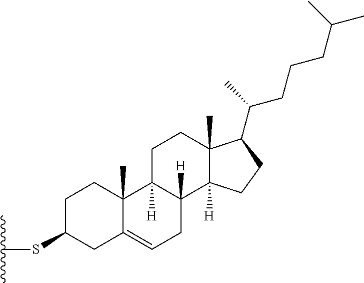

a substituted by t occurrences of R₇.

In some instances, t is 0. In some instances, m is 4. In some instances, m is 8.

Also featured herein is a structurally stabilized peptide conjugate represented by Formula VI or a pharmaceutically acceptable salt thereof, wherein Formula VI is defined by:

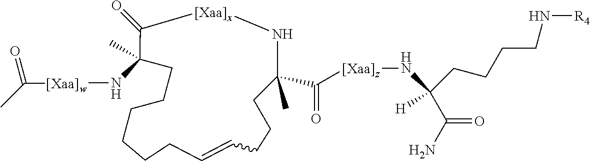

wherein
R₄ is —C(O)—(CH₂CH₂)—[O—CH₂CH_{2]8}—N(H)C(O)—(CH₂)—R₆;

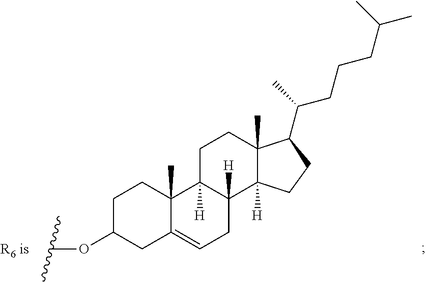

[Xaa]_{w} is
(SEQ ID NO: 35)
DISGINASVVNIQ;
[Xaa]ₓ is
(SEQ ID NO: 36)
EIDRLN;
and
[Xaa]_{z} is
(SEQ ID NO: 77)
VAKNLNESLIDLQELG.

Also featured herein is a pharmaceutical compound comprising any one of the structurally-stabilized peptides, pharmaceutically acceptable salts thereof, or the conjugates as previously described, and a pharmaceutically acceptable carrier.

Further, provided herein are methods of making and using any one of the structurally-stabilized peptides, pharmaceutically acceptable salts thereof, or the conjugates. In some instances, provided herein are methods of treating a coronavirus infection in a human subject in need thereof, the method comprising administering to the human subject a therapeutically-effective amount of any one of the structurally-stabilized peptides, the pharmaceutically acceptable salts thereof, or the conjugates previously described (e.g., comprising SEQ ID NO:7-21; e.g., comprising SEQ ID NO:10 or any one of SEQ ID NO:13, 17, or 20). Also provided herein are methods of preventing a coronavirus infection in a human subject in need thereof, the method comprising administering to the human subject a therapeutically-effective amount of any one of the structurally-stabilized peptides, the pharmaceutically acceptable salts thereof, or the conjugates previously described (e.g., comprising SEQ ID NO:7-21; e.g., comprising SEQ ID NO:10 or any one of SEQ ID NO:13, 17, or 20).

Further featured herein are methods of treating a coronavirus infection in a subject in need thereof, the method comprising administering to the subject a therapeutically-effective amount of any one of the structurally-stabilized peptides, the pharmaceutically acceptable salts thereof, or the conjugates previously described (e.g., comprising SEQ ID NO:7-21; e.g., comprising SEQ ID NO:10 or any one of SEQ ID NO: 13, 17, or 20). Also disclosed herein are methods of preventing a coronavirus infection in a subject in need thereof, the method comprising administering to the subject a therapeutically-effective amount of any one of the structurally-stabilized peptides, the pharmaceutically acceptable salts thereof, or the conjugates previously described (e.g., comprising SEQ ID NO:7-21; e.g., comprising SEQ ID NO:10 or any one of SEQ ID NO:13, 17, or 20). In some instances, the subject is selected from a cow, a pig, a horse, a cat, a dog, a rat, a mouse, or a bat.

In some instances, the coronavirus infection is by a betacoronavirus. In some instances, the coronavirus infection is by an alphacoronavirus.

In some instances, the coronavirus infection is caused by an infection by SARS-CoV-2. In some instances, the coronavirus infection is caused by an infection by a variant of SARS-CoV-2. In some instances, the variant is selected from Wuhan-Hu-1, B.1.427/B.1.429, B.1.617.2, D614G B.1, or Brazilian variant P.1. In some instances, the variant is an omicron variant. In some instances, the variant is selected from B.1.351, Cluster 5, Lineage B.1.1.207, Lineage B.1.1.7, Variant of Concern 202102/02, Lineage B.1.1.317, Lineage B.1.1.318, Lineage B.1.351, Lineage B.1.429, Lineage B.1.525, Lineage P.1 (also known as Lineage B.1.1.28), Lineage B.1.1.529, Lineage BA.1, Lineage BA.1.1, Lineage BA.2, Lineage BA.3, Lineage BA.4 Lineage BA.5, D614G, E484K, N501Y, S477G/N, or P681H. In some instances, the variant includes one of Wuhan-Hu-1, B.1.427/B.1.429, B.1.617.2, D614G B.1, Brazilian variant P.1, B.1.1.7, B.1.351, B.1.525, B.1.526, B.1.617.1, B.1.617.3, P.2, B.1.621, B.1.621.1, B.1.1.529, BA.1, BA.1.1, BA.2, BA.3, BA.4 or BA.5.

The methods disclosed herein also include methods of making one of the structurally-stabilized peptides. In some instances, the methods of making the structurally-stabilized peptide include (a) providing a peptide having the sequence set forth in SEQ ID NO: 6 or a variant thereof (e.g., with at least one to eighteen of the amino acids at positions 3, 4, 6, 9, 11, 13, 15, 18, 20, 22, 25, 27, 29, 32, 34, 35, 37, or 38 in SEQ ID NO: 6 are substituted), and (b) cross-linking the peptide, and optionally purifying the structurally-stabilized peptide. In some instances, cross-linking the peptide is by a ruthenium catalyzed metathesis reaction. In some instances, the methods further include formulating the structurally-stabilized peptide as a sterile pharmaceutical composition.

Also provided herein are methods of synthesizing one of the conjugates provided herein. In some instances, the methods of synthesizing one of the conjugates provided herein include (a) providing the structurally-stabilized polypeptide; and (b) derivatizing a resin bound amine of the structurally-stabilized polypeptide with PEG and/or cholesterol containing a carboxylic acid on a resin. In some instances, the methods of synthesizing one of the conjugates provided herein include (a) providing the structurally-stabilized polypeptide; and (b) derivatizing a resin bound amine of the structurally-stabilized polypeptide with PEG and/or thiocholesterol containing a carboxylic acid on a resin.

Also provided herein are methods of synthesizing a heptad repeat domain 2 (HR2) polypeptide. In some instances, the methods include (a) providing the HR2 polypeptide; and (b) derivatizing a resin bound amine of the HR2 polypeptide with PEG and/or cholesterol containing a carboxylic acid on a resin. In some instances, the methods include (a) providing the HR2 polypeptide; and (b) derivatizing a resin bound amine of the HR2 polypeptide with PEG and/or thiocholesterol containing a carboxylic acid on a resin.

In some instances, the derivatizing step comprises incorporating the carboxy thiocholesterol or carboxy cholesterol by solid phase synthesis by the steps of: dissolving thiocholesterol in dichloromethane (DCM) or cholesterol in tetrahydrofuran (THF), thereby generating a solution; and adding, in order, a base, t-butyl ester of bromoacetic acid, and trifluoroacetic acid to the solution. In some instances, the derivatizing step further comprises treating the structurally-stabilized polypeptide bound to the resin with piperidine in a solution comprising dimethylformamide (DMF); capping the N-terminus of the structurally-stabilized polypeptide with acetic anhydride; deprotecting the C-terminus of the structurally-stabilized polypeptide with hydrazine in DMF; acylating the structurally-stabilized polypeptide with an Fmoc-protected PEG(n) amino acid; crosslinking the structurally-stabilized polypeptide; and isolating the structurally-stabilized polypeptide from the resin.

In some instances, n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances, n=4, 5, 6, 7, or 8. In some cases, n=4, 5, 6, 7, 8, 10, 12, 14, 16, 18, 20, 22, 24, 26, 28, 30, 32, 34, or 36. In some instances, the C-terminal lysine of SEQ ID NO: 6 is substituted with a resin-bound amine, optionally wherein the C-terminal lysine of SEQ ID NO: 6 is further substituted with a resin-bound carboxylic acid or thiol. In some instances, the cholesterol is thiocholesterol. In some instances, the conjugate comprises PEG(n)-cholesterol, wherein n is 1-36, optionally wherein n is 4, 5, 6, 7, or 8. In some cases, n=4, 5, 6, 7, 8, 10, 12, 14, 16, 18, 20, 22, 24, 26, 28, 30, 32, 34, or 36. In some instances, the conjugate comprises PEG(n)-thiocholesterol, wherein n is 1-36, optionally wherein n is 4, 5, 6, 7, or 8. In some cases, n=4, 5, 6, 7, 8, 10, 12, 14, 16, 18, 20, 22, 24, 26, 28, 30, 32, 34, or 36. In some instances, cross-linking the peptide is by a ruthenium catalyzed metathesis reaction.

Also featured herein is a method for synthesizing a stabilized peptide, wherein the method comprises the step of subjecting a peptide comprising Formula VII to ring closing metathesis conditions to provide a stabilized peptide, wherein Formula VII is defined by:

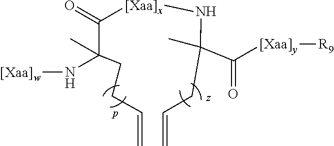

or a pharmaceutically acceptable salt thereof, wherein:

[Xaa]_{w} is
(SEQ ID NO: 35)
DISGINASVVNIQ;
[Xaa]ₓ is
(SEQ ID NO: 36)
EIDRLN;
[Xaa]_{y} is
(SEQ ID NO: 37)
VAKNLNESLIDLQELGK;

R₉ is —OH or

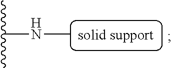 [SMILES: *NC]

p is 2, 3, 4, 5, 6, 7, or 8; and
z is 2, 3, 4, 5, or 6;
wherein optionally the amino group of the N-terminal aspartic acid in [Xaa]_{w} is replaced with —N(H)C(O)—(C₁₋₄ alkyl).

In some instances, the ring closing metathesis conditions comprise exposing said peptide comprising Formula IV to a Grubbs ring closing metathesis ruthenium catalyst.

In some instances, the stabilized peptide comprises Formula III, which is defined by:

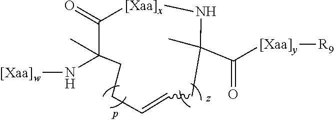

or a pharmaceutically acceptable salt thereof, wherein:

[Xaa]_{w} is
(SEQ ID NO: 35)
DISGINASVVNIQ;
[Xaa]ₓ is
(SEQ ID NO: 36)
EIDRLN;
[Xaa]_{y} is
(SEQ ID NO: 37)
VAKNLNESLIDLQELGK;

R₉ is —OH or

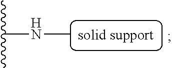 [SMILES: *NC]

p is 2, 3, 4, 5, 6, 7, or 8; and
z is 2, 3, 4, 5, or 6;
wherein optionally the amino group of the N-terminal aspartic acid in [Xaa]_{w} is replaced with —N(H)C(O)—(C₁₋₄ alkyl).

In some instances, p is 5. In some instances, z is 3. In some instances, the method further comprises derivatizing the C-terminus end of the stabilized peptide with a moiety comprising a polyethylene glycol moiety and a cholesterol or thiocholesterol moiety. In some instances, the method further comprises derivatizing the C-terminus end of the stabilized peptide to install a **—C(O)—(C₂₋₆ alkylene)-[O—CH₂CH₂]ₘ—N(R₅)C(O)—(C₁₋₆ alkylene)-R₆ group, wherein ** is the point of attachment to the amino group in the side chain of the C-terminal lysine in [Xaa]_{y}; R₅ is hydrogen or C₁₋₄ alkyl; R₆ is one of the following:

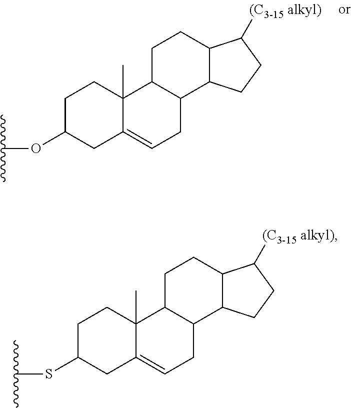

optionally wherein each of which is substituted by t occurrences of R₇;
R₇ represents independently for each occurrence C₁₋₃ alkyl, hydroxyl, or C₁₋₃ alkoxyl;
   wherein m is 3, 4, 5, 6, 7, 8, 9, 10, 11, 12, 13, 14, 15, or 16; and
t is 0, 1, 2, or 3.

In some instances, R₆ is

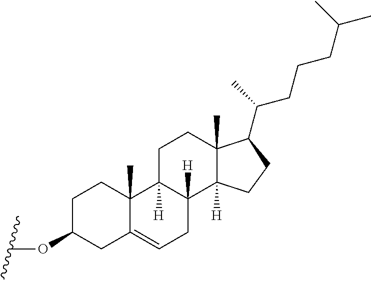

substituted by t occurrences of R₇.

In some instances, R₆ is

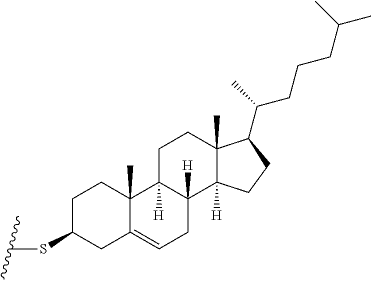

substituted by t occurrences of R₇.

In some instances, t is 0. In some instances, m is 4. In some instances, m is 8. In some instances, R₉ is

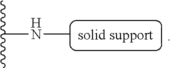 [SMILES: *NC]

In some instances, the method further comprises the step of cleaving off the solid support.

Also featured herein are nanoparticle compositions. In some instances, the nanoparticle compositions include any of the structurally-stabilized peptides (e.g., SEQ ID Nos. 10, 13, 17, 20), the pharmaceutically acceptable salts thereof, or the conjugates provided herein. In some instances, the nanoparticle composition is a PLGA nanoparticle. In some instances, the nanoparticle composition includes a lactic acid:glycolic acid ratio of the PLGA nanoparticle in the range of 2:98 to 100:0. In some instances, the nanoparticle composition also includes chitosan, a dextrin, or both.

Also featured herein is a peptide linker comprising a PEG(n)-thiocholesterol having the formula:

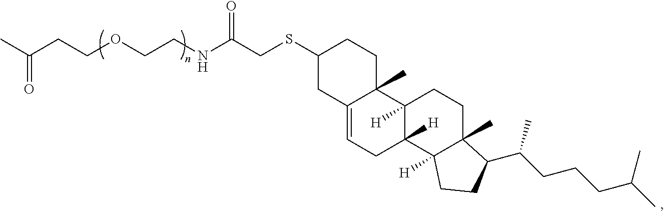

wherein n is 1-36.

Next, featured herein is a peptide linker comprising a PEG(n)-cholesterol comprising the formula:

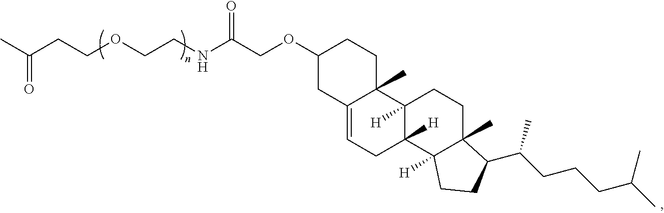

wherein n is 1-36.

In some instances, n of the peptide linkers is 4, 5, 6, 7, or 8. In some cases, n=4, 5, 6, 7, 8, 10, 12, 14, 16, 18, 20, 22, 24, 26, 28, 30, 32, 34, or 36. In some instances, the peptide linker includes a lysine affixed to the PEG.

Also featured herein is a compound having one of the following formulae:

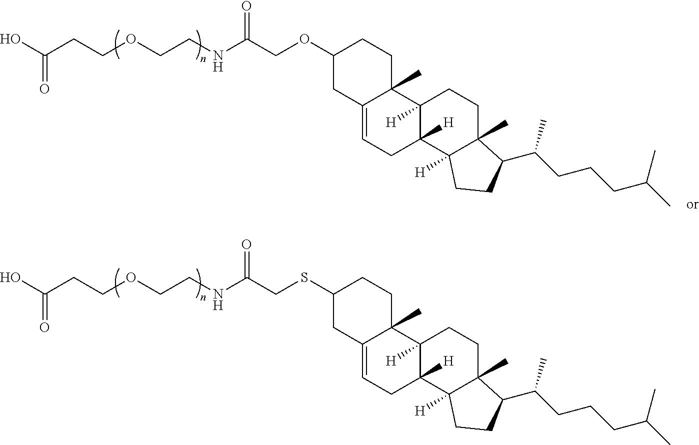

wherein n is 1-36. In some instances, n is 4, 5, 6, 7, or 8. In some cases, n=4, 5, 6, 7, 8, 10, 12, 14, 16, 18, 20, 22, 24, 26, 28, 30, 32, 34, or 36.

In some instances, for any of the structurally-stabilized peptides or conjugates described herein, 8=(R)-α-(7′-octenyl)alanine or (R)-α-(4′-pentenyl)alanine; and X=(S)-α-(4′-pentenyl)alanine or (S)-α-(7′-octenyl)alanine. In some instances, 8=(R)-α-(7′-octenyl)alanine; and X=(S)-α-(4′-pentenyl)alanine.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the disclosure will be apparent from the following detailed description and from the claims.

### BRIEF DESCRIPTION OF DRAWINGS

FIG. 1 depicts a mechanism of action of SARS-CoV-2 S fusion inhibitor peptides.

FIG. 2 provides the amino acid sequence of the S protein (SEQ ID NO: 1) of SARS-CoV-2.

FIG. 3 is a schematic representation of the SARS-CoV-2 spike (S) protein, including the sequence composition of the heptad repeat domain 1 (HRT) (SEQ ID NO: 2) and heptad repeat domain 2 (HR2) (SEQ ID NO: 3) fusion domains.

FIG. 4 shows an alignment of the HRT and HR2 regions of SARS-CoV-2 and SARS-CoV-1 (“SARS, C.2004”) viruses highlighting the sequence homology, with striking sequence identity between the HR2 regions of SARS-CoV-2 and SARS-CoV-1.

FIG. 5 shows a variety of non-natural amino acids containing olefinic tethers that can be used to generate hydrocarbon stapled SARS-CoV-2 S peptides bearing staples spanning i, i+3; i, i+4; and i, i+7 positions. Single staple scanning is used to generate a library of singly stapled SARS-CoV-2-19 HR2 peptides for conjugation to PEG-thiocholesterol or PEG-cholesterol moieties.

FIG. 6 shows a variety of staple compositions in multiply stapled peptides and staple scanning to generate a library of multiply stapled SARS-CoV-2 HR2 peptides for conjugation to PEG-thiocholesterol or PEG-cholesterol moieties.

FIG. 7 shows a variety of staple compositions in tandem stitched peptides to generate a library of stitched SARS-CoV-2 HR2 peptides for conjugation to PEG-thiocholesterol or PEG-cholesterol moieties.

FIG. 8 is an illustration of an exemplary approach to designing, synthesizing, and identifying optimal stapled peptide constructs to target the SARS-CoV-2 fusion apparatus, including the generation of Ala scan, staple scan, and variable N- and C-terminal deletion, addition, and derivatization libraries for conjugation to PEG-thiocholesterol or PEG-cholesterol moieties. Singly and doubly stapled and stitched constructs, including alanine and staple and stitch scans, are used to identify optimal stapled peptides for conjugation to PEG-thiocholesterol or PEG-cholesterol moieties and application in in vitro and in vivo analyses.

FIG. 9 is a helical wheel depiction of a portion of the SARS-CoV-2 HR2 domain structured as an amphipathic alpha-helix (SEQ ID NO: 4), illustrating the predominantly hydrophobic binding interface, with flanking charged or polar residues at the perimeter of the binding interface and at the non-interacting face. The arrow refers to the hydrophobic moment.

FIG. 10 shows a synthetic schema for converting thiocholesterol or cholesterol into a carboxylic acid for facile on-resin derivatization of stapled peptides with cholesterol-containing moieties. DCM: dichloromethane; TFA: trifluoroacetic acid.

FIG. 11A-11B shows a synthetic schema of the steps for on-resin derivatization of the stapled peptide sequence (SEQ ID NO: 74) with a PEG-linked thiocholesterol moiety (FIG. 11A) and a synthetic schema of the steps for on-resin derivatization of the stapled peptide sequence (SEQ ID NO: 74) with a PEG-linked cholesterol moiety (FIG. 11B).

FIG. 12 shows exemplary unstapled (SEQ ID NOs: 5 and 6) and structurally-stabilized SARS-CoV-2 HR2 peptide sequences (SEQ ID NOs: 7-21) generated by single i, i+7 staple scanning of a core template sequence (amino acids 1178-1199) bearing N- and C-terminal sequence extensions (e.g., amino acids 1168-1205) and C-terminal derivatization with PEG-thiocholesterol or PEG-cholesterol moieties of varying PEG linker length. 8=(R)-α-(7′-octenyl)alanine; and X=(S)-α-(4′-pentenyl)alanine.

FIG. 13 shows that an unstapled HR2 sequence of SEQ ID NO: 5 exhibits no antiviral activity against the Wuhan-Hu-1 fluorescent pseudovirus corresponding to GenBank QHD43416.1, as measured by IXM microscopy. In contrast, C-terminal derivatization of SEQ ID NO: 5 with a PEG4-thiocholesterol moiety to produce the peptide of SEQ ID NO: 6 yields dose-responsive antiviral activity (pseudovirus: Wuhan-Hu-1; cells: 293T-ACE2; peptides: serial 2-fold dilution starting at 5 μM; read-out: 72 h).

FIG. 14 shows that a stapled HR2 peptide bearing a PEG4-thiocholesterol moiety appended on-resin (Staple D, SEQ ID NO: 10) exhibits consistent and potent anti-viral activity in the pseudovirus assay whether the cells were treated with peptide (1 μM) before or after viral inoculation across a series of SARS-CoV-2 pseudovirus variants. In contrast, the corresponding unstapled peptide (SEQ ID NO: 6) is ineffective when applied after viral inoculation and exhibits less anti-viral activity compared to the stapled sequence even when applied before viral inoculation with a series of SARS-CoV-2 pseudovirus variants (pseudovirus: D614G B.1, Wuhan-Hu-1, B.1.526, B.1.427/B.1.429, B.1.1.7; cells: 293T-ACE2; read-out taken at 72 h).

FIG. 15 shows the differential antiviral activity of an unstapled and stapled HR2 peptides bearing a PEG4-thiocholesterol moiety appended on-resin, with peptide of SEQ ID NO: 10 (Staple D) showing the most potent dose-responsive anti-viral activity, followed by the unstapled peptide of SEQ ID NO: 6. The stapled peptide of SEQ ID NO: 17 (Staple K) was the least active in this pseudovirus assay (pseudovirus: B.1.526; cells: 293T-ACE2; serial 2-fold dilution starting at 1 μM; read-out: 72 h).

FIG. 16 shows the differential antiviral activity of a series of i, i+7 stapled HR2 peptides bearing a PEG4-thiocholesterol moiety appended on-resin. Whereas peptides of SEQ ID NOs: 11, 14, and 15 (Staples E, H, I, respectively) show little to no activity and peptides of SEQ ID NOs: 13 and 16 (Staples G and J, respectively) exhibit moderate activity, the peptide of SEQ ID NO: 10 (Staple D) stands out as having uniquely potent activity among the various stapled HR2 peptides in the SARS-CoV-2 pseudovirus assay (pseudovirus: D614G B.1; cells: 293T-ACE2; peptide doses of 100, 300, 1000 nM; read-out at 48 h).

FIG. 17 shows the differential antiviral activity of a series of i, i+7 stapled HR2 peptides bearing a PEG4-thiocholesterol moiety appended on-resin. Whereas peptides of SEQ ID NOs: 11, 14, and 15 (Staples E, H, I, respectively) show little to no activity (as measured by concentration of virus in μM on the X-axis) and peptides of SEQ ID NOs: 13 and 16 (Staples G and J) exhibit moderate activity, the peptide of SEQ ID NO: 10 (Staple D), consistent with the pseudovirus assays results shown in FIG. 15, stands out as having uniquely potent activity among the various stapled HR2 peptides in this SARS-CoV-2 live virus assay (live virus: USA-WA1/2020; cells: VeroB6; peptide dose-range 4-1000 nM (i.e., each group of bars, from bottom to top, represent a decreasing 2-fold dilution of peptide; i.e., 1000 nM, 500 nM, 250 nM, 125 nM, 62.5 nM, 31.25 nM, 15.625 nM, 7.8125 nM, 3.90625 nM).

FIGS. 18A-18B shows the differential antiviral activity of an i, i+7 staple scan of the indicated HR2 peptide sequence (SEQ ID NOs: 7-21) bearing a C-terminal PEG4-thiocholesterol moiety against SARS-CoV-2 (virus: live Beta strain; cells: VeroB6; peptide dose 4 μM), with the most active peptide sequences noted with an asterisk (FIG. 18A). A discrete subset of staple positions afford potent anti-viral activity (SEQ ID NOs: 10, 13, 17, 20), as summarized on a helical wheel depiction of the helical portion of the HR2 sequence (FIG. 18B).

FIG. 19 shows the antiviral activity of an i, i+7 stapled HR2 peptide of SEQ ID NO:10 bearing a PEG4-thiocholesterol moiety against a GFP expressing SARS-CoV-2 Omicron variant B.1.1.529.1 (BA1) pseudovirus (cells; 293T-ACE2 cells; peptide serial 2-fold dilution from 2000 nM; 48 h read-out).

FIG. 20 shows the antiviral activity of an i, i+7 stapled HR2 peptide of SEQ ID NO:10 bearing a PEG4-thiocholesterol moiety against live SARS-CoV-2 beta and delta strains (cells: VeroB6; peptide dose-range 15-4000 nM (i.e., each group of bars, from bottom to top, represent a decreasing 2-fold dilution of peptide; i.e., 4000 nM, 2000 nM, 1000 nM, 500 nM, 250 nM, 125 nM, 62.5 nM, 31.25 nM, 15.625 nM).

FIG. 21 shows the antiviral activity of an i, i+7 stapled HR2 peptide of SEQ ID NO:13 bearing a PEG4-thiocholesterol moiety, as measured against GFP-expressing SARS-CoV pseudoviruses, including SARS-CoV-2 Wuhan-hu-1, SARS-CoV-2 Omicron BA1.1.529.1 (BA1), SARS-CoV-2 Omicron BA1.1.529.2 (BA2), and SARS-CoV-1 (Urbani) (cells; 293T-ACE2 cells; peptide serial 2-fold dilution from 10 PM; 48 h read-out).

FIG. 22 shows the antiviral activity of an i, i+7 stapled HR2 peptide of SEQ ID NO:20 bearing a PEG4-thiocholesterol moiety, as measured against GFP-expressing SARS-CoV pseudoviruses, including SARS-CoV-2 Wuhan-hu-1, SARS-CoV-2 Omicron BA1.1.529.1 (BA1), SARS-CoV-2 Omicron BA1.1.529.2 (BA2), and SARS-CoV-1 (Urbani) (cells; 293T-ACE2 cells; peptide serial 2-fold dilution from 10 PM; 48 h read-out).

FIG. 23 shows the antiviral activity of i, i+7 stapled HR2 peptides of SEQ ID NO: 13 and SEQ ID NO: 20 bearing a PEG4-thiocholesterol moiety, as measured against live SARS-CoV-2 beta strain virus (cells: VeroB6; 4 M dosing).

FIGS. 24A-24B shows a sequence map of an i, i+7 stapled HR2 peptide of SEQ ID NO: 10 (Staple D spanning positions K1181 and E1188), highlighting the amino acid positions that are alternatively in contact with the HR1 core or unbound (solvent exposed) (FIG. 24A). Mutagenesis studies revealed a series of exemplary positions that are relatively unaffected by substitution of the native residue with alanine, as assessed in the context of SEQ ID NO: 10 with a C-terminal PEG4 thiocholesterol moiety against GFP-expressing SARS-CoV-1 (Urbani) in a pseudovirus assay (cells; 293T-ACE2 cells; peptide 4-fold serial dilution from 1.25 μM; 48 h read-out) (FIG. 24B).

FIGS. 25A-25E shows the differential antiviral activity of a series of i, i+7 stapled HR2 peptides bearing a PEG-thiocholesterol moiety of variable PEG length (n=3-8). The peptide of SEQ ID NO: 10 (Staple D) bearing a PEG8 linker moiety exhibits the most potent, dose-responsive activity in this pseudovirus assay across a series of five SARS-CoV-2 variants (as measured by number of green cells or percent GFP positive shown on the X-axis), with PEG3 having comparatively less activity among the constructs of variable PEG-linker length (pseudoviruses/variants: Wuhan-Hu-1 (FIG. 25A), B.1.427/B.1.429 (FIG. 25B), B.1.617.2 (FIG. 25C), D614G B.1(FIG. 25D), Brazilian variant P.1 (FIG. 25E); cells: 293T-ACE2; serial 2-fold dilution starting at 1 μM (i.e., from bottom to top for each cluster of bars: 1 μM, 500 nM, 250 nM, 125 nM, 62.5 nM, 31.25 nM, 15.625 nM, 7.8125 nM); read-out: 48 h).

FIG. 26 shows the differential antiviral activity of a series of i, i+7 stapled HR2 peptides bearing a PEG-thiocholesterol moiety of variable PEG length (n=3-8) (Staple D, SEQ ID NO: 10). The peptide of SEQ ID NO: 10 (Staple D) bearing a PEG8 linker moiety exhibits the most potent, dose-responsive activity in this SARS-CoV-2 live virus assay, with PEG3 having comparatively less activity among the constructs of variable PEG-linker length (live virus: S. African B.1.351; cells: VeroB6; peptide dose-range 4-1000 nM; i.e., serial 2-fold dilution, from bottom to top for each cluster of bars: 1000 nM, 500 nM, 250 nM, 125 nM, 62.5 nM, 31.25 nM, 15.625 nM, 7.8125 nM, 3.90625 nM).

FIG. 27 shows the differential antiviral activity of a series of i, i+7 stapled HR2 peptides of SEQ ID NO:10 bearing a PEG-thiocholesterol moiety of variable PEG length (n=0, 3-20), as measured against GFP expressing SARS-CoV-2 Omicron variant B.1.1.529.1 (BA1) (cells: 293T-ACE2; serial 3-fold dilution starting at 3.3 μM from bottom to top for each cluster of bars: 3.3 μM, 1.1 μM, 367 nM, 122 nM, 41 nM, 13 nM, 4.5 nM, 1.5 nM; read-out: 48 h).

FIG. 28 shows the differential antiviral activity of a series of i, i+7 stapled HR2 peptides of SEQ ID NO:10 bearing a PEG-thiocholesterol moiety of variable PEG length (n=0, 3-20), as measured against GFP expressing SARS-CoV-1 (Urbani) (cells: 293T-ACE2; serial 2-fold dilution starting at 2 μM from bottom to top for each cluster of bars: 2 μM, 1 μM, 500 nM, 250 nM, 125 nM, 62.5 nM, 31.2 nM, 15.6 nM; read-out: 48 h).

FIG. 29 shows the differential antiviral activity of a series of i, i+7 stapled HR2 peptides of SEQ ID NO:10 bearing a PEG-thiocholesterol moiety of variable PEG length (n=0, 3-20), as measured against live SARS-CoV-2 Beta strain virus (cells: VeroB6; peptide dose-range 7.8-1000 nM; i.e., serial 2-fold dilution, from bottom to top for each cluster of bars: 4000 nM, 2000 nM, 1000 nM, 500 nM, 250 nM, 125 nM, 62.5 nM, 31.2 nM, 15.6 nM, 7.8 nM).

FIG. 30 shows that the series of i, i+7 stapled HR2 peptides bearing a PEG-thiocholesterol moiety of variable PEG length (n=3-8) (Staple D, SEQ ID NO: 10) exhibit no non-specific anti-viral activity against a Vesicular Stomatitis Virus (VSV) pseudovirus with murine leukemia virus (MLV) core, whereas the corresponding unstapled peptide (SEQ ID NO: 6) demonstrates some non-specific anti-viral activity (pseudovirus: VSV; cells: 293T-ACE2; peptides, 2.5 μM; read-out: 48 h).

FIG. 31 shows that an unstapled peptide derivatized with PEG4-thiocholesterol on resin (SEQ ID NO: 6) exhibits somewhat improved anti-viral activity compared to an unstapled peptide derivatized in solution with a GSGSGC-PEG4-cholesterol moiety (shown in SEQ ID NO:71) in this SARS-CoV-2 live virus assay (live virus: S. African B.1.351; cells: VeroB6; peptide dose-range 4-1000 nM with two-fold dilutions).

FIG. 32 shows that an unstapled HR2 peptide (SEQ ID NO: 6) and a stapled HR2 peptide (SEQ ID NO: 10) derivatized with PEG4-thiocholesterol on resin show no non-specific cytotoxicity when applied to 293T-ACE2 cells in the pseudovirus assay, whereas the corresponding unstapled HR2 peptide derivatized in solution with a GSGSGC-PEG4-cholesterol moiety kills cells within the dosing range (pseudovirus: D614G B.1; cells: 293T-ACE2; serial 2-fold dilution starting at 2500 nM (from bottom to top, 2500 nM, 1250 nM, 630 nM, 315 nM, 158 nM, 78 nM); read-out: 48 h).

FIG. 33 shows a direct fluorescence polarization binding curve of SEQ ID NO: 10 derivatized at the C-terminus with PEG8-Chol and at the N-terminus with a FITC-β-Ala in place of the acetyl, combined with a serial dilution of a recombinant five-helix bundle (5HB) lacking the 3^{rd} HR2 group. Addition of the FITC-HR2 peptide completes the fusogenic six helix bundle. (peptide: 5 nM; 5-HB protein serial dilution from 1000 nM).

FIG. 34 shows the differential antiviral activity of SEQ ID NO: 10 derivatized at the C-terminus with PEG8-Chol against a collection of GFP expressing SARS-CoV-2 variant pseudoviruses (293T-ACE2 cells; peptide serial dilution from 1000 nM; 48 h read-out).

FIG. 35 shows the differential antiviral activity of SEQ ID NO: 10 derivatized at the C-terminus with PEG8-Chol against GFP expressing SARS-CoV-2 Omicron variant pseudoviruses (293T-ACE2 cells; peptide serial dilution from 250 nM; 48 h read-out).

FIG. 36 shows the differential antiviral activity of SEQ ID NO: 10 derivatized at the C-terminus with PEG8-Chol against SARS-CoV-2 beta and delta live viruses (cells: Vero; peptide serial dilution from 100 nM serial dilution of peptide starting at 100 nM; 48 hour read-out).

FIG. 37 shows the antiviral activity of SEQ ID NO: 10 derivatized at the C-terminus with PEG8-Chol against GFP expressing Alphacoronavirus NL63 pseudovirus (293T-ACE2 cells; peptide serial 3-fold dilution from 10 μM; 48 h read-out).

### DETAILED DESCRIPTION

The present disclosure is based, inter alia, on the discovery that stabilized (e.g., stapled) peptides may be designed to selectively bind to one or more coronaviruses (e.g., betacoronaviruses such as SARS-CoV-2). Accordingly, the present disclosure provides novel methods (e.g., approaches to convert cholesterol/thiocholesterol into carboxylic acids for on-resin derivatization) and compositions (e.g., peptides, stabilized peptides, combinations of peptides; combinations of stabilized peptides; combinations of peptides and stabilized peptides; and their cholesterol conjugates) for treating, for developing treatments for, and for preventing infection with one or more coronaviruses (e.g., betacoronaviruses such as SARS-CoV-2). Thus, the peptides and composition disclosed herein can be used to prevent and/or treat COVID-19.

### Coronavirus Peptides

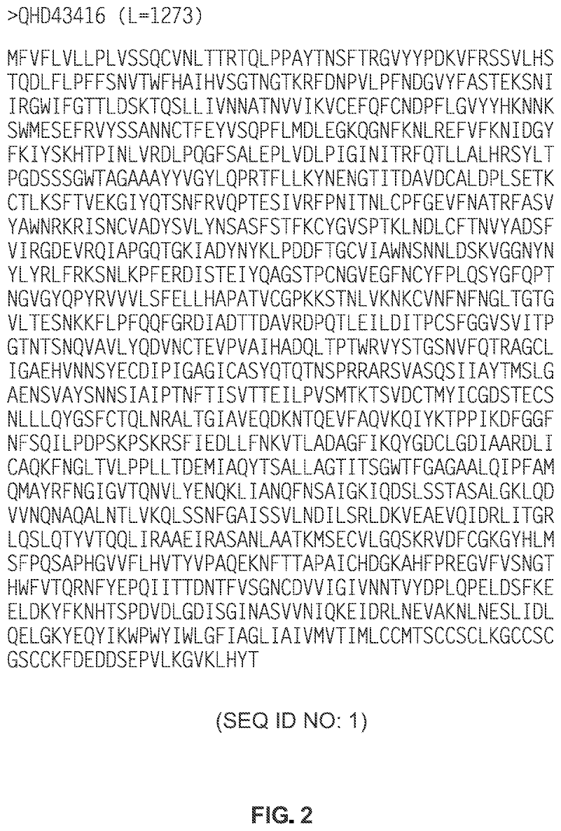

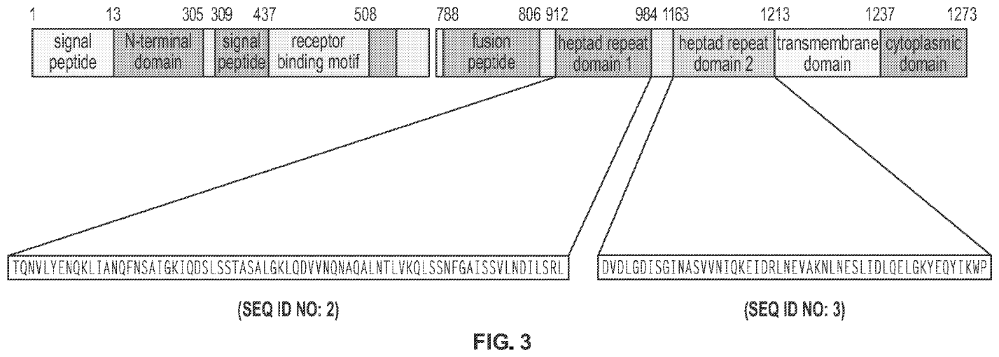

The amino acid sequence of an exemplary coronavirus surface glycoprotein is provided in FIG. 2. (See also, GenBank Accession No. QHD43416.1.) An exemplary amino acid sequence of the heptad repeat domain 1 (HR1) in SARS-CoV-2 S is shown as SEQ ID NO: 2 in FIG. 3. An exemplary amino acid sequence of the heptad repeat domain 2 (HR2) in SARS-CoV-2 S is also shown as SEQ ID NO: 3 in FIG. 3.

Other exemplary amino acid sequences of the HR2 in SARS-CoV-2 S are provided as SEQ ID NOs: 5 and 6 in Table 1.

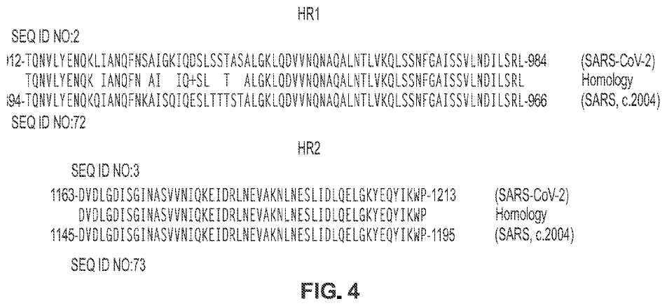

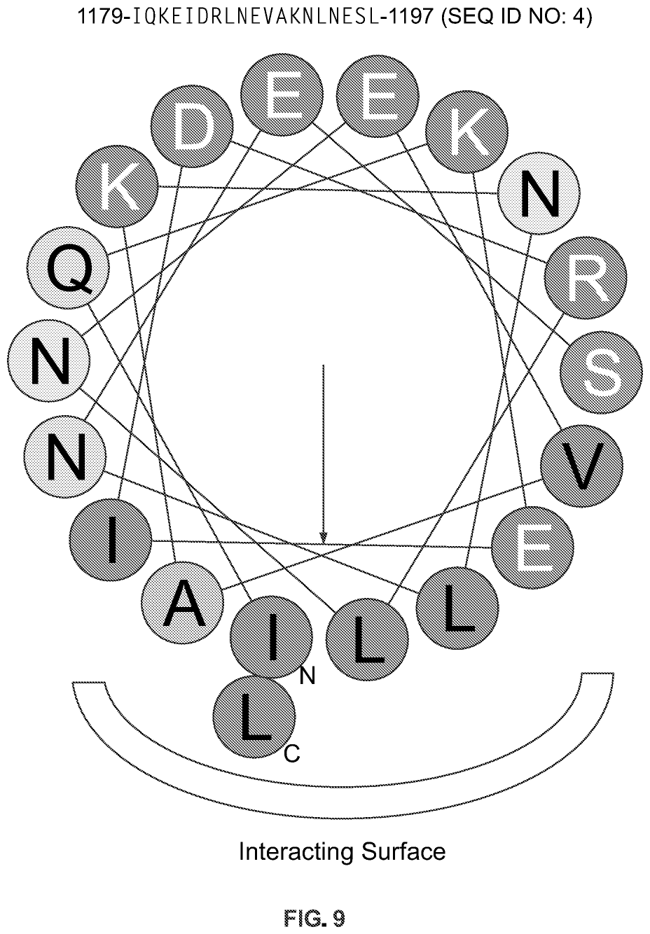

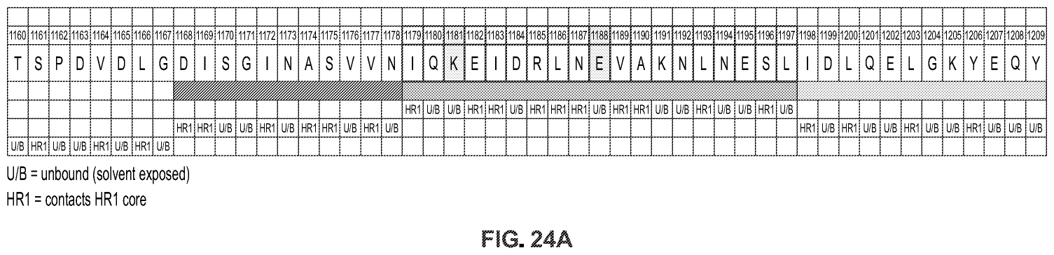

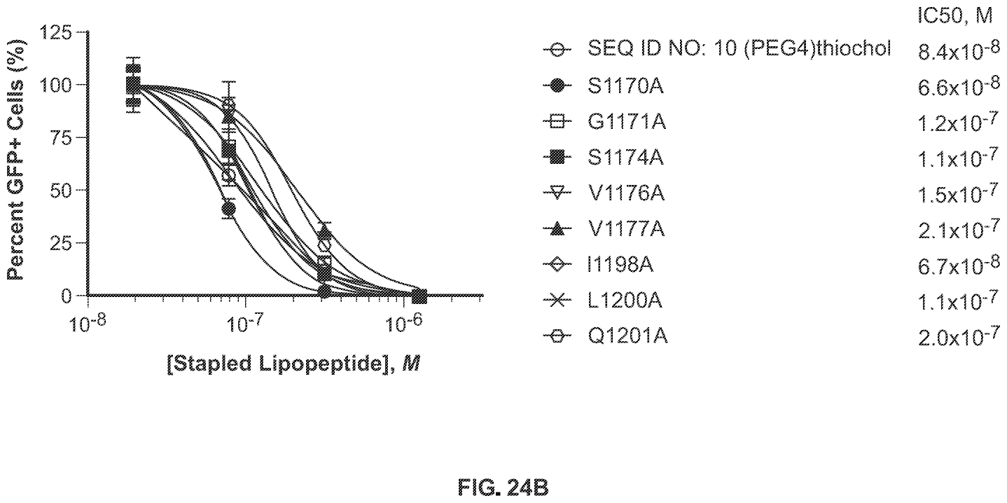

In certain instances, the SARS-CoV-2 HR1 or HR2 peptides described herein (e.g., SEQ ID NOs: 5 and 6) may also contain one or more (e.g., 1, 2, 3, 4, 5, 6, 7, 8, 9, 10, 11, 12, 13, 14, 15, 16, 17, or 18) amino acid substitutions (relative to an amino acid sequence set forth in any one of SEQ ID NOs: 5 and 6), e.g., one or more (e.g., 1, 2, 3, 4, 5, 6, 7, 8, 9, 10, 11, 12, 13, 14, 15, 16, 17, or 18) conservative and/or non-conservative amino acid substitutions. In addition, in some instances at least two (e.g., 2, 3, 4, 5, or 6) amino acids of SEQ ID NOs: 5 and 6 may be substituted by α, α-disubstituted non-natural amino acids with olefinic side chains. The type of substitutions that are made can, e.g., be guided by an alignment of the HR2-like region of two SARS sequences, SARS-CoV-1 and SARS-CoV-2 (FIG. 4). The guidance provided in the Structurally-Stabilized Peptides section below regarding the amino acids that can be varied is equally relevant for the peptides described herein. Residues that are unchanged between SARS-CoV-1 and SARS-CoV-2 in such an alignment are either unmodified or substituted with a non-natural amino acid or a conservative amino acid. Residues in the alignment that are found replaced by conservative substitutions (e.g., Isoleucine in SARS-CoV-1 and SARS-CoV-2 replaced by Leucine or Methionine) in the HR2-like region of SARS-CoV-1 and SARS-CoV-2 are either not replaced or replaced by conservative amino acid substitutions. Residues that are not conserved between the HR2-like region of SARS-CoV-1 and SARS-CoV-2 can be replaced by any amino acid. In some instances, residues that are conserved between the HR2-like region of SARS-CoV-1 and SARS-CoV-2 but are located on the non-interacting face of the HR2 helix can be replaced by any amino acid (see, e.g., FIGS. 9, 24A, and 24B. In some instances, the mutations in SEQ ID NO: 6 are not made at positions 14 and 21 of SEQ ID NO: 6. In some instances, the mutations in SEQ ID NO: 6 are not made at positions 17 and 24 of SEQ ID NO: 6. In some instances, the mutations in SEQ ID NO: 6 are not made at positions 20 and 27 of SEQ ID NO: 6. In some instances, the mutations in SEQ ID NO: 6 are not made at positions 21 and 28 of SEQ ID NO: 6. In some instances, the mutations in SEQ ID NO: 6 are not made at positions 24 and 31 of SEQ ID NO: 6.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine).

In some instances, the SARS-CoV-2 HR2 peptides described herein (e.g., SEQ ID NOs: 5 or 6) may also contain at least one, at least 2, at least 3, at least 4, or at least 5 amino acids added to the N-terminus of the peptide. In some instances, the SARS-CoV-2 HR2 peptides described herein (e.g., SEQ ID NO: 5 or 6) may also contain at least one, at least 2, at least 3, at least 4, or at least 5 amino acids added to the C-terminus of the peptide. In some instances, the SARS-CoV-2 HR2 peptides described herein (e.g., SEQ ID NO: 5 or 6) may also contain at least one, at least 2, at least 3, at least 4, or at least 5 amino acids deleted at the N-terminus of the peptide. In some instances, the SARS-CoV-2 HR2 peptides described herein (e.g., SEQ ID NO: 5 or 6 may also contain at least one, at least 2, at least 3, at least 4, or at least 5 amino acids deleted at the C-terminus of the peptide.

In some cases, the peptides are lipidated. In some cases, the peptides are modified to comprise polyethylene glycol and/or cholesterol. In some cases, the peptides (e.g., SEQ ID NOs.: 5 or 6) include the following formula affixed to the C-terminus of the peptide:

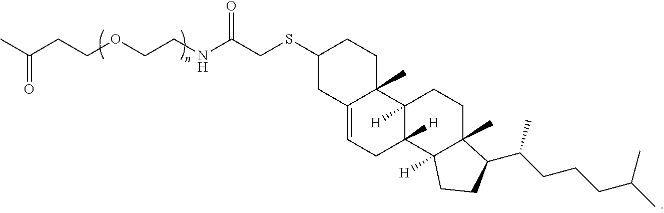

In some cases, the aforementioned formula is affixed through a modifiable carbon atom to the C-terminus of the peptide. In some cases, the peptides (e.g., SEQ ID NOs.: 5 or 6) include the following formula affixed to the C-terminus of the peptide:

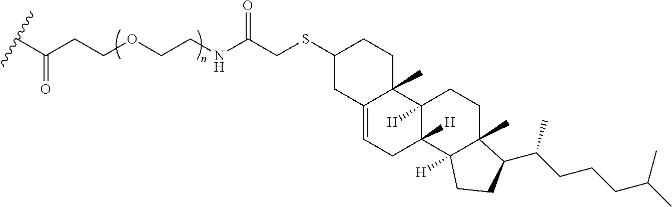

In some cases, the peptides (e.g., SEQ ID NOs.: 5 or 6) include the following formula affixed to the C-terminus of the peptide:

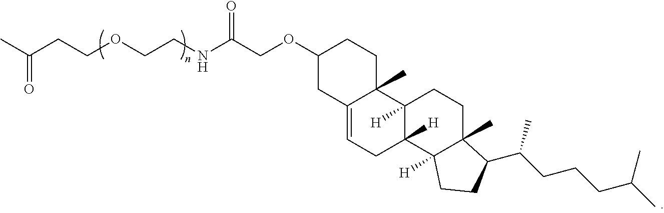

In some cases, the aforementioned formula is affixed through a modifiable carbon atom to the C-terminus of the peptide. In some cases, the peptides (e.g., SEQ ID NOs.: 5 or 6) include the following formula affixed to the C-terminus of the peptide:

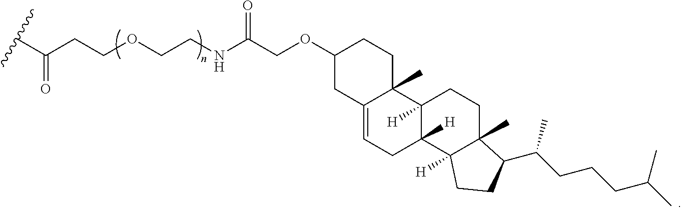

In some instances, n in the above formula is n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances, n=4. In some instances, n=8. In some instance, the sulfur atom in the formula is replaced with an oxygen atom.

In some instances, the peptides described herein comprise an amino acid sequence that is at least 55%, 60%, 65%, 70%, 75%, 80%, 85%, 90%, or 94% identical to sequence set forth in SEQ ID NO:6 (DISGINASVVNIQKEIDRLNEVAKNLNESLIDLQELGK). In some instances, a peptide as described above (i) is alpha-helical; (ii) is protease resistant; (iii) inhibits fusion of SARS-CoV-2 with a host cell; and/or (iv) inhibits infection of a cell by SARS-CoV-2. In some instances, the peptides inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

In some instances, the peptides include an amino acid sequence that has 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, or 2 substitutions, insertions, and/or deletions relative to SEQ ID NO: 6. In some instances, the peptides include 1, 2, 3, 4, 5, 6, 7, 8, 9, 10, 11, 12, 13, 14, 15, 16, 17, or 18 substitutions, insertions, and/or deletions relative to SEQ ID NO: 6. In some instances, a peptide having substitutions, insertions, and/or deletions relative to SEQ ID NO: 6 as described above (i) is alpha-helical; (ii) is protease resistant; (iii) inhibits fusion of SARS-CoV-2 with a host cell; and/or (iv) inhibits infection of a cell by SARS-CoV-2. In some instances, the peptides inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

In some instances, the peptide is 36 to 50 (e.g., 36, 37, 38, 39, 40, 41, 42, 43, 44, 45, 46, 47, 48, 49, or 50) amino acids in length. In instances in which the peptide is modified to comprise polyethylene glycol and/or cholesterol, the peptide is 19 to 50 (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In some instances, the peptide ranges from 19 to 100, 38 to 100, 19 to 60, 38 to 60, 19 to 50, 38 to 50, 19 to 45, 38 to 45, 19 to 40 and 38 to 40 amino acids in length.

In some instances, the peptides described above (i) are alpha-helical; (ii) are protease resistant; (iii) inhibit fusion of SARS-CoV-2 with a host cell; and/or (iv) inhibit infection of a cell by SARS-CoV-2. In some instances, the peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

### Structurally-Stabilized Peptides

Disclosed herein are stapled SARS-CoV-2 peptides based on a portion of the HR2 region. In some instances, the stapled SARS-CoV-2 peptides are derived from SARS-CoV-2 HR2(1168-1205) (DISGINASVVNIQKEIDRLNEVAKNLNESLIDLQELGK (SEQ ID NO:6)). In some instances, the stapled SARS-CoV-2 peptides derived from SEQ ID NO:9 include SAH-SARS-CoV-2-A; SAH-SARS-CoV-2-B; SAH-SARS-CoV-2-C; SAH-SARS-CoV-2-D; SAH-SARS-CoV-2-E; SAH-SARS-CoV-2-F; SAH-SARS-CoV-2-G; SAH-SARS-CoV-2-H; SAH-SARS-CoV-2-I; SAH-SARS-CoV-2-J; SAH-SARS-CoV-2-K; SAH-SARS-CoV-2-L; SAH-SARS-CoV-2-M; SAH-SARS-CoV-2-N; or SAH-SARS-CoV-2-0 (e.g., SEQ ID NOs: 7-21), as shown in Table 1 below.

TABLE 1
Stapled SARS-CoV-2 HR2 Peptides.

| | Sequence (X and/or 8 refer to α, | |
|---|---|---|
| | α- disubstituted non-natural amino | |
| | acids with olefinic side chains | |
| | whose side chains are cross-linked | SEQ |
| | to form an internally cross-linked | ID |
| Name | peptide) | NO |
| SARS- | DISGINASVVNIQKEIDRLNEVAKNLNESLIDLQEL | 5 |
| CoV-2 | | |
| HR2(₁₁₆₈₋₁₂₀₃) | | |
| SARS- | DISGINASVVNIQKEIDRLNEVAKNLNESLIDLQEL* | 22 |
| CoV-2 | | |
| HR2(₁₁₆₈₋₁₂₀₃) | | |
| SARS- | DISGINASVVNIQKEIDRLNEVAKNLNESLIDLQELG | 23 |
| CoV-2 | | |
| HR2(₁₁₆₈₋₁₂₀₄) | | |
| SARS- | DISGINASVVNIQKEIDRLNEVAKNLNESLIDLQELG* | 24 |
| CoV-2 | | |
| HR2(₁₁₆₈₋₁₂₀₄) | | |
| SARS- | DISGINASVVNIQKEIDRLNEVAKNLNESLIDLQELGK | 6 |
| CoV-2 | | |
| HR2(₁₁₆₈₋₁₂₀₅) | | |
| SARS- | DISGINASVVNIQKEIDRLNEVAKNLNESLIDLQELGK* | 25 |
| CoV-2 | | |
| HR2(₁₁₆₈₋₁₂₀₅) | | |
| SAH- | DISGINASVVS8IQKEIDXLNEVAKNLNESLIDLQELGK | 7 |
| SARS- | | |
| CoV-2- | | |
| A | | |
| SAH- | DISGINASVVN8QKEIDRXNEVAKNLNESLIDLQELGK | 8 |
| SARS- | | |
| CoV-2- | | |
| B | | |
| SAH- | DISGINASVVNI8KEIDRLXEVAKNLNESLIDLQELGK | 9 |
| SARS- | | |
| CoV-2- | | |
| C | | |
| SAH- | DISGINASVVNIQ8EIDRLNXVAKNLNESLIDLQELGK | 10 |
| SARS- | | |
| CoV-2- | | |
| D | | |
| SAH- | DISGINASVVNIQK8IDRLNEXAKNLNESLIDLQELGK | 11 |
| SARS- | | |
| CoV-2- | | |
| E | | |
| SAH- | DISGINASVVNIQKE8DRLNEVXKNLNESLIDLQELGK | 12 |
| SARS- | | |
| CoV-2- | | |
| F | | |
| SAH- | DISGINASVVNIQKEI8RLNEVAXNLNESLIDLQELGK | 13 |
| SARS- | | |
| CoV-2- | | |
| G | | |
| SAH- | DISGINASVVNIQKEID8LNEVAKXLNESLIDLQELGK | 14 |
| SARS- | | |
| CoV-2- | | |
| H | | |
| SAH- | DISGINASVVNIQKEIDR8NEVAKNXNESLIDLQELGK | 15 |
| SARS- | | |
| CoV-2-I | | |
| SAH- | DISGINASVVNIQKEIDRL8EVAKNLXESLIDLQELGK | 16 |
| SARS- | | |
| CoV-2-J | | |
| SAH- | DISGINASVVNIQKEIDRLN8VAKNLNXSLIDLQELGK | 17 |
| SARS- | | |
| CoV-2- | | |
| K | | |
| SAH- | DISGINASVVNIQKEIDRLNE8AKNLNEXLIDLQELGK | 18 |
| SARS- | | |
| CoV-2- | | |
| L | | |
| SAH- | DISGINASVVNIQKEIDRLNEV8KNLNESXIDLQELGK | 19 |
| SARS- | | |
| CoV-2- | | |
| M | | |
| SAH- | DISGINASVVNIQKEIDRLNEVA8NLNESLXDLQELGK | 20 |
| SARS- | | |
| CoV-2- | | |
| N | | |
| SAH- | DISGINASVVNIQKEIDRLNEVAK8LNESLIXLQELGK | 21 |
| SARS- | | |
| CoV-2- | | |
| O | | |

In Table 1, “8”=(R)-α-(7′-octenyl)alanine; “X”=(S)-α-(4′-pentenyl)alamine; and *=PEG-thiocholesterol or PEG-cholesterol moiety. In some instances, *=the one of the two formulae shown below, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances, n=4. In some instances, n=8. The two formulae indicated by the “*” include:

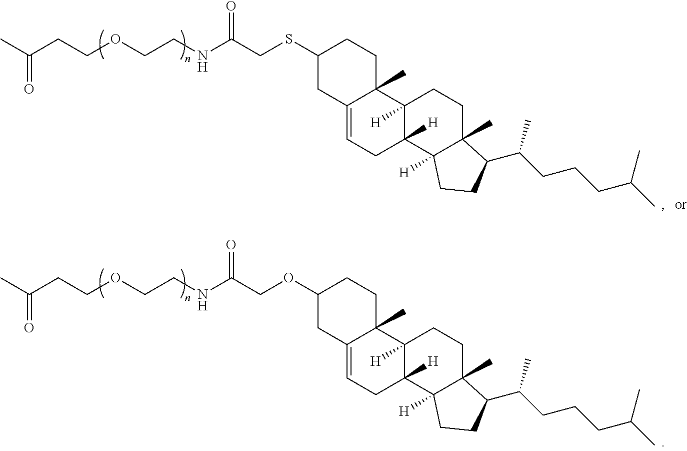

In some cases, the two formulae indicated by the “*” include:

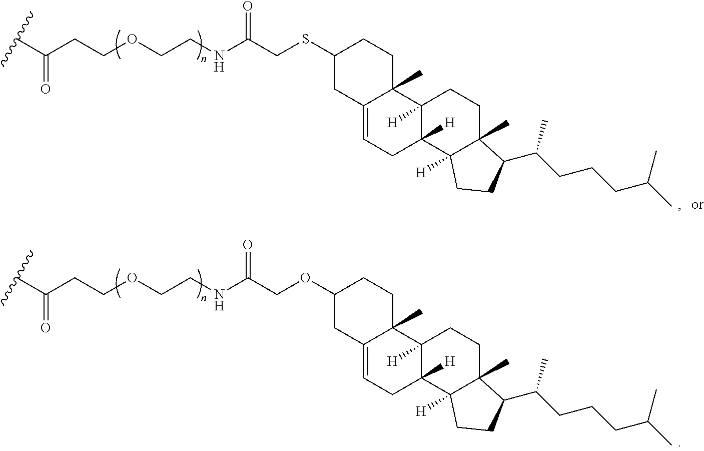

It should be understood that the above peptides can be modified to include additional amino acids (e.g., 1, 2, 3, 4, 5, 6, 7, 8, 9, 10 amino acids added) at the N and/or C-terminus, and/or to have N and/or C terminal deletions (e.g., 1, 2, 3, 4, 5, 6, 7, 8, 9, 10 amino acids deleted). In some instances, the stapled SARS-CoV-2 peptides are derived from SEQ ID NO:4.

Note that the bolded and underlined sequence used herein (e.g., in Table 1) identifies the stapling amino acids at the N- and C-termini and the intervening sequence between staples for each disclosed peptide. In some instances (e.g., SEQ ID NOs: 7-21), the structurally-stabilized peptide is single-stapled peptide.

In some instances, SEQ ID NO: 6 includes one or more variants. For instance, if positions 25 or 29 of SEQ ID NO: 6 are substituted, they are substituted by an α, α-disubstituted non-natural amino acid with olefinic side chains, or are substituted by any amino acid. In some instances, if one or more of positions 1, 2, 5, 7, 8, 10, 12, 16, 17, 19, 23, 24, 26, 28, 30, 31, 33, and 36 of SEQ ID NO: 6 are substituted, they are substituted by conservative amino acid substitutions. In some instances, if one or more of positions 3, 4, 6, 9, 11, 13, 15, 18, 20, 22, 27, 32, 34, 35, 37, or 38 in SEQ ID NO: 6 are substituted, they are substituted by any amino acid.

The disclosure encompasses each and every peptide and structurally stabilized peptide listed in Table 1 as well as variants thereof. In some instances, the structurally stabilized peptide is 19 to 50 (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In some instances, the structurally stabilized peptide is 19 to 60 (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60) amino acids in length. In some instances, the structurally stabilized peptide ranges from 19 to 100, 38 to 100, 19 to 60, 38 to 60, 19 to 50, 38 to 50, 19 to 45, 38 to 45, 19 to 40 and 38 to 40 amino acids in length. In some instances, the structurally stabilized peptide described above have one or more (1, 2, 3, 4, 5, 6) of the properties listed below: (i) is alpha-helical; (ii) is protease resistant; (iii) inhibits fusion of SARS-CoV-2 with a host cell; and/or (iv) inhibits infection of a cell by SARS-CoV-2. In some instances, the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

In some instances, the structurally stabilized peptide include an amino acid sequence that has 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, or 2 substitutions, insertions, and/or deletions relative to SEQ ID NO: 6, SEQ ID NO:10, SEQ ID NO:13, SEQ ID NO:17, or SEQ ID NO:20. In some instances, a structurally stabilized peptide having substitutions, insertions, and/or deletions relative to SEQ ID NO: 6, SEQ ID NO:10, SEQ ID NO:13, SEQ ID NO:17, or SEQ ID NO:20 as described above (i) is alpha-helical; (ii) is protease resistant; (iii) inhibits fusion of SARS-CoV-2 with a host cell; and/or (iv) inhibits infection of a cell by SARS-CoV-2. In some instances, the structurally stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally stabilized peptide binds to a polypeptide comprising or consisting of the sequence of SEQ ID NO:78.

In some instances, disclosed herein are peptides that comprise 0-10 (0, 1, 2, 3, 4, 5, 6, 7, 8, 9, 10) amino acid substitutions compared to one of the single-stapled peptides (e.g., SEQ ID NOs: 7-21) in Table 1. In some instances, disclosed herein are peptides that are at least 75% (e.g., at least 75%, at least 80%, at least 85%, at least 90%, at least 94%, at least 95% identical) to one of the single-stapled peptides (e.g., SEQ ID NOs: 7-21) in Table 1. In some instances, the structurally stabilized peptide is 19 to 50 (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In some instances, the structurally stabilized peptide is 19 to 60 (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60) amino acids in length. In some instances, the structurally stabilized peptide ranges from 19 to 100, 38 to 100, 19 to 60, 38 to 60, 19 to 50, 38 to 50, 19 to 45, 38 to 45, 19 to 40 and 38 to 40 amino acids in length. In some instances, the structurally stabilized peptide described above have one or more (1, 2, 3, 4, 5, 6) of the properties listed below: (i) is alpha-helical; (ii) is protease resistant; (iii) inhibits fusion of SARS-CoV-2 with a host cell; and/or (iv) inhibits infection of a cell by SARS-CoV-2. In some instances, the structurally stabilized peptides inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally stabilized peptide binds to a polypeptide comprising or consisting of the sequence of SEQ ID NO:78.

In some instances, the stapled peptide is a peptide comprising or consisting of any one of the amino acids sequences of SEQ ID NOs: 5 or 6, except that at least two (e.g., 2, 3, 4, 5, 6) amino acids of SEQ ID NOs: 5 or 6 are replaced with a non-natural amino acid capable of forming a staple. In some instances, the non-natural amino acid is an α, α-disubstituted non-natural amino acids with olefinic side chains. In some instances, the stapled peptide is a peptide comprising or consisting of any one of the amino acids sequences of SEQ ID NOs: 5 or 6, except that at least two (e.g., 2, 3, 4, 5, 6) amino acids of SEQ ID NOs: 5 or 6 are replaced with a non-natural amino acid capable of forming a staple. In some instances, the non-natural amino acid is an α, α-disubstituted non-natural amino acids with olefinic side chains. In some instances, the structurally stabilized peptide is 19 to 50 (e.g., 36, 37, 38, 39, 40, 41, 42, 43, 44, 45, 46, 47, 48, 49, or 50) amino acids in length. In some instances, the structurally stabilized peptide is 19 to 60 (e.g., 36, 37, 38, 39, 40, 41, 42, 43, 44, 45, 46, 47, 48, 49, 50, 51, 52, 53, 54, 55, 56, 57, 58, 59, or 60) amino acids in length. In some instances, the structurally stabilized peptide ranges from 19 to 100, 38 to 100, 19 to 60, 38 to 60, 19 to 50, 38 to 50, 19 to 45, 38 to 45, 19 to 40 and 38 to 40 amino acids in length. In some instances, the structurally stabilized peptide described above have one or more (1, 2, 3, 4, 5, 6) of the properties listed below: (i) is alpha-helical; (ii) is protease resistant; (iii) inhibits fusion of SARS-CoV-2 with a host cell; and/or (iv) inhibits infection of a cell by SARS-CoV-2. In some instances, the structurally stabilized peptides inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally stabilized peptide binds to a polypeptide comprising or consisting of the sequence of SEQ ID NO:78.

In some instances, disclosed herein are peptides that comprise 0-10 (0, 1, 2, 3, 4, 5, 6, 7, 8, 9, 10) amino acid substitutions compared to one of the unmodified peptides (e.g., SEQ ID NOs: 5 or 6) in Table 1. In some instances, disclosed herein are peptides that are at least 75% (e.g., at least 75%, at least 80%, at least 85%, at least 90%, at least 95% identical) to one of the unmodified peptides (e.g., SEQ ID NOs: 5 or 6) in Table 1. In some instances, the substitution as described herein is a conservative substitution. In some instances, the structurally stabilized peptide is 19 to 50 (e.g., 36, 37, 38, 39, 40, 41, 42, 43, 44, 45, 46, 47, 48, 49, or 50) amino acids in length. In some instances, the structurally stabilized peptide is 19 to 60 (e.g., 36, 37, 38, 39, 40, 41, 42, 43, 44, 45, 46, 47, 48, 49, 50, 51, 52, 53, 54, 55, 56, 57, 58, 59, or 60) amino acids in length. In some instances, the structurally stabilized peptide ranges from 19 to 100, 38 to 100, 19 to 60, 38 to 60, 19 to 50, 38 to 50, 19 to 45, 38 to 45, 19 to 40 and 38 to 40 amino acids in length. In some instances, the structurally stabilized peptide described above have one or more (1, 2, 3, 4, 5, 6) of the properties listed below: (i) is alpha-helical; (ii) is protease resistant; (iii) inhibits fusion of SARS-CoV-2 with a host cell; and/or (iv) inhibits infection of a cell by SARS-CoV-2. In some instances, the structurally stabilized peptides inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally stabilized peptide binds to a polypeptide comprising or consisting of the sequence of SEQ ID NO:78.

In some instances, any substitution as described herein can be a conservative substitution. In some instances, any substitution as described herein is a non-conservative substitution.

In some instances, the non-natural amino acids that may be used as stapling amino acids are: (R)-2-(2′-propenyl)alanine; (R)-2-(4′-pentenyl)alanine; (R)-α-(7′-octenyl)alanine; (S)-α-(2′-propenyl)alanine; (S)-α-(4′-pentenyl)alanine; (S)-2-(7′-octenyl)alanine; α,α-Bis(4′-pentenyl)glycine; and α,α-Bis(7′-octeny)glycine.

In some embodiments, an internal staple replaces the side chains of 2 amino acids, i.e., each staple is between two amino acids separated by, for example, 6 amino acids. In some embodiments, the amino acids forming the staple are at each of positions i and i+7 of the staple. For example, where a peptide has the sequence . . . X1, X2, X3, X4, X5, X6, X7, X8, X9 . . . , cross-links between X1 and X8 (i and i+7) are useful hydrocarbon stapled forms of that peptide. The use of an i and i+4 staple, multiple cross-links (e.g., 2, 3, 4, or more), or a tandem stitch is also contemplated. Additional description regarding making and use of hydrocarbon-stapled peptides can be found, e.g., in U.S. Patent Publication Nos. 2012/0172285, 2010/0286057, and 2005/0250680, the contents of all of which are incorporated by reference herein in their entireties.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). The structural-stabilization may be by, e.g., stapling the peptide (see, e.g., Walensky, J. Med. Chem., 57:6275-6288 (2014), the contents of which are incorporated by reference herein in its entirety). In some cases, the staple is a hydrocarbon staple.

In some instances, a staple used herein is a lactam staple; a UV-cycloaddition staple; an oxime staple; a thioether staple; a double-click staple; a bis-lactam staple; a bis-arylation staple; or a combination of any two or more thereof. Stabilized peptides as described herein include stapled peptides as well as peptides containing multiple staples or any other chemical strategies for structural reinforcement (see. e.g., Balaram P. Cur. Opin. Struct. Biol. 1992; 2:845; Kemp D S, et al., J Am. Chem. Soc. 1996; 118:4240; Omer B P, et al., J Am. Chem. Soc. 2001; 123:5382; Chin J W, et al., Int. Ed. 2001; 40:3806; Chapman R N, et al., J Am. Chem. Soc. 2004; 126:12252; Home W S, et al., Chem., Int. Ed. 2008; 47:2853; Madden et al., Chem Commun (Camb). 2009 Oct. 7; (37): 5588-5590; Lau et al., Chem. Soc. Rev., 2015, 44:91-102; and Gunnoo et al., Org. Biomol. Chem., 2016, 14:8002-8013; each of which is incorporated by reference herein in its entirety).

A peptide is “structurally-stabilized” in that it maintains its native secondary structure. For example, stapling allows a peptide, predisposed to have an α-helical secondary structure, to maintain its native α-helical conformation. This secondary structure increases resistance of the peptide to proteolytic cleavage and heat, and may increase target binding affinity, hydrophobicity, plasma membrane binding, and/or cell permeability. Accordingly, the stapled (cross-linked) peptides described herein have improved biological activity and pharmacology relative to a corresponding non-stapled (un-cross-linked) peptide.

In certain instances, the modification(s) to introduce structural stabilization (e.g., internal cross-linking, e.g., stapling) into the SARS-CoV-2 HR2 peptides described herein may be positioned on the face of the SARS-CoV-2 HR2 helix that does not interact with the recombinant 5-helix bundle of SARS-CoV-2 or corresponding native fusion apparatus. Alternatively, the modification(s) to introduce stabilization (e.g., internal cross-linking, e.g., stapling) into the SARS-CoV-2 HR2 peptides described herein may be positioned on the face of the SARS-CoV-2 HR2 helix that does interact with the 5 helix bundle of SARS-CoV-2. In some cases, a SARS-CoV-2 HR2 peptide described herein is stabilized by introducing a staple (e.g., a hydrocarbon staple) at the interface of the interacting and non-interacting helical faces of the SARS-CoV-2 HR2 protein. In some cases, a SARS-CoV-2 HR2 peptide described herein is stabilized by introducing a staple (e.g., a hydrocarbon staple) or staples at the border between the hydrophobic interacting surface and the non-interacting faces of the SARS-CoV-2 HR2 protein.

In some instances, the modifications to introduce structural stabilization (e.g., internal cross-linking, e.g., stapling) into the SARS-CoV-2 HR2 peptides described herein are positioned at the amino acid positions in the SARS-CoV-2 HR2 peptide corresponding to residues:
(i) 11 and 18 of SEQ ID NO: 6;
(ii) 12 and 19 of SEQ ID NO: 6;
(iii) 13 and 20 of SEQ ID NO: 6;
(iv) 14 and 21 of SEQ ID NO: 6;
(v) 15 and 22 of SEQ ID NO: 6;
(vi) 16 and 23 of SEQ ID NO: 6;
(vii) 17 and 24 of SEQ ID NO: 6;
(viii) 18, and 25 of SEQ ID NO: 6;
(ix) 19 and 26 of SEQ ID NO: 6;
(x) 20 and 27 of SEQ ID NO: 6;
(xi) 21 and 28 of SEQ ID NO: 6;
(xii) 22 and 29 of SEQ ID NO: 6;
(xiii) 23 and 30 of SEQ ID NO: 6;
(xiv) 24 and 31 of SEQ ID NO: 6; or
(xv) 25 and 32 of SEQ ID NO: 6;

In certain instances, the SARS-CoV-2 HR2 peptides described herein (e.g., SEQ ID NOs: 5 or 6) may also contain one or more (e.g., 1, 2, 3, 4, or 5) amino acid substitutions (relative to an amino acid sequence set forth in any one of SEQ ID NOs: 5 or 6), e.g., one or more (e.g., 1, 2, 3, 4, or 5) conservative and/or non-conservative amino acid substitutions. In some instances, the SARS-CoV-2 HR2 peptides described herein (e.g., SEQ ID NOs: 5 or 6) may also contain at least one, at least 2, at least 3, at least 4, or at least 5 amino acids added to the N-terminus of the peptide. In some instances, the SARS-CoV-2 HR2 peptides described herein (e.g., SEQ ID NOs: 5 or 6) may also contain at least one, at least 2, at least 3, at least 4, or at least 5 amino acids added to the C-terminus of the peptide.

In some instances, the N-terminal aspartic acid in any one of the peptides disclosed herein is replaced with —N(H)C(O)—(C₁₋₄ alkyl). In some instances, the carboxylic acid group of the C-terminal lysine in any one of the peptides disclosed herein is replaced with C(O)NH₂. In some instances, the N-terminal aspartic acid in SEQ ID NO: 6, SEQ ID NO:10, or SEQ ID NO:20 is replaced with —N(H)C(O)—(C₁₋₄ alkyl). In some instances, the carboxylic acid group of the C-terminal lysine in SEQ ID NO: 6, SEQ ID NO:10,SEQ ID NO:13, SEQ ID NO:17, or SEQ ID NO:20 is replaced with C(O)NH₂.

In some instances, the C-terminal lysine of any of the peptides discloses herein is replaced with omithine (e.g., L-omithine), L-2,3-diaminopropionic acid, L-2,7-diaminoheptanoic acid, diamino butyric acid (e.g., L-2,4-diamino butyric acid, an amino acid having an alpha carbon amine, or a diamine.

In one aspect, the structurally-stabilized SARS-CoV-2 HR2 peptide comprises Formula (I),

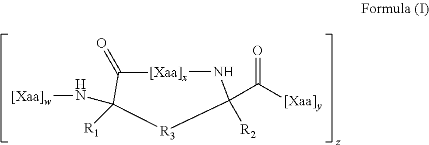

or a pharmaceutically acceptable salt thereof, wherein:
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄—K—R₄]n; each of which is substituted with 0-6 R₅;
R₄ is alkyl, alkenyl, or alkynyl;
R₅ is halo, alkyl, OR₆, N(R₆)₂, SR₆, SOR₆, SO₂R₆, C₀₂R₆, R₆, a fluorescent moiety, or
a radioisotope;
K is O, S, SO, SO₂, CO, CO₂, CONR₆, or

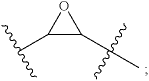 [SMILES: *C1OC1*]

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);
and each Xaa is independently an amino acid; and
   wherein the structurally-stabilized peptide wherein the peptide (i) inhibits fusion of SARS-CoV-2 with a host cell; and/or (ii) inhibits infection of a cell by SARS-CoV-2. In some instances, the structurally stabilized peptides inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In some instances, the structurally stabilized peptide binds to a polypeptide comprising or consisting of the sequence of SEQ ID NO:78.

In another aspect, the structurally-stabilized SARS-CoV-2 HR2 peptide comprises Formula (I-A). Formula (I-A) is defined by:

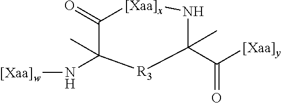

or a pharmaceutically acceptable salt thereof. In some instances, the structurally stabilized peptide consists of Formula (I-A).

In some instances, R₃ of Formula (I-A) is alkenylene. In some instances, the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In another aspect, the structurally stabilized peptide comprises Formula Ia, which is defined by:

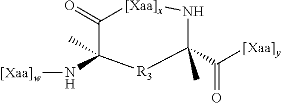

or a pharmaceutically acceptable salt thereof. In one aspect, the structurally stabilized peptide consists of Formula (Ia). In some instances, the amino group of the N-terminal aspartic acid in [Xaa]_{w} of Formula (Ia) is replaced with —N(H)C(O)—(C₁₋₄ alkyl), and optionally the carboxylic acid group of the C-terminal lysine in [Xaa]_{y} is replaced with —C(O)NH₂.

In some instances, the structurally stabilized peptide comprises or consists of

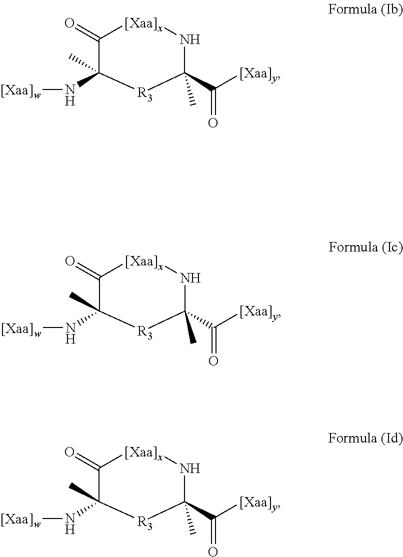

or a pharmaceutically acceptable salt thereof.

In some instances, the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In some instances, R₃ of any one of Formular (I)—(Id) (i.e., I, Ia, Ib, Ic, Id) is C₇₋₁₅ alkenylene. In some instances, R₃ is C₉₋₁₃ alkenylene. In some instances, R₃ is C₁₁ alkenylene. In some instances, R₃ is —(CH₂)₃₋₇—CH═CH—(CH₂)₃₋₇—. In some instances, R₃ is —(CH₂)₅₋₇—CH═CH—(CH₂)₃₋₄—. In some instances, R₃ is —(CH₂)₆—CH═CH—(CH₂)₃—.

In some instances, the amino group of the N-terminal aspartic acid in [Xaa]W is replaced with —N(H)C(O)—(C₁₋₄ alkyl). In some instances, the amino group of the N-terminal aspartic acid in [Xaa]_{w} is replaced with —N(H)C(O)CH₃. In some instances, the carboxylic acid group of the C-terminal lysine in [Xaa]_{y} is replaced with —C(O)NH₂

In another aspect, discloses herein is a structurally stabilized peptide conjugate comprising or consisting of Formula II, wherein Formula II is defined by:

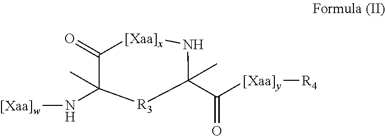

or a pharmaceutically acceptable salt thereof.

In some instances, R₃ of Formula II is alkenylene. In some instances, R₄ is **—C(O)—(C₂₋₆ alkylene)-[O—CH₂CH₂]ₘ—N(R₅)C(O)—(C₁₋₆ alkylene)-R₆, wherein ** is the point of attachment to the amino group in the side chain of the C-terminal lysine in [Xaa]_{y}. In some instances, R₅ is hydrogen or C₁₋₄ alkyl.

In some instances, R₆ is one of the following:

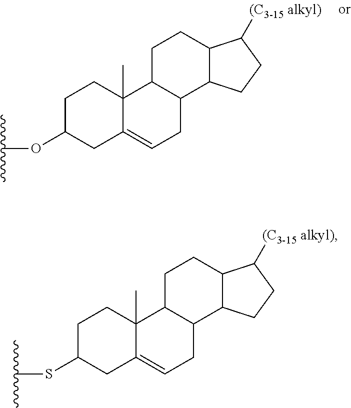

optionally wherein each of which is substituted by t occurrences of R₇;

In some instances, R₇ represents independently for each occurrence C₁₋₃ alkyl, hydroxyl, or C₁₋₃ alkoxyl. In some instances, m is 3, 4, 5, 6, 7, 8, 9, 10, 11, 12, 13, 14, 15, or 16. In some instances, t is 0, 1, 2, or 3.

In some instances, the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

In some instances, the structurally stabilized peptide comprises or consists of Formula IIa, which is defined by

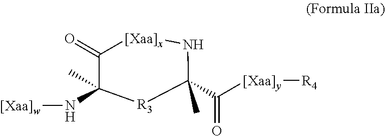

or a pharmaceutically acceptable salt thereof, wherein optionally the amino group of the N-terminal aspartic acid in [Xaa]_{w} is replaced with —N(H)C(O)—(C₁₋₄ alkyl). In some instances, the carboxylic acid group of the C-terminal lysine in [Xaa]_{y} is replaced with —C(O)NH₂.

In some instances, the structurally stabilized peptide comprises or consists of

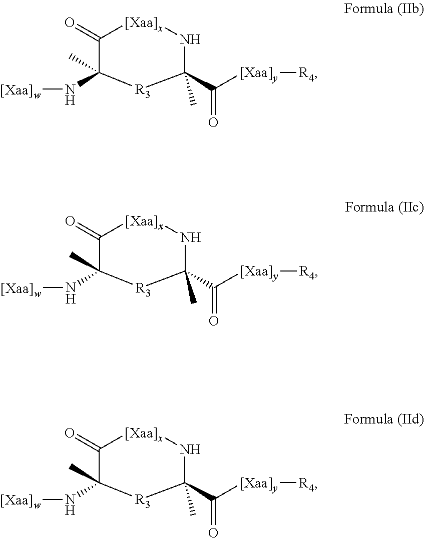

or a pharmaceutically acceptable salt thereof. In some instances, the amino group of the N-terminal aspartic acid in [Xaa]_{w} is replaced with —N(H)C(O)—(C₁₋₄ alkyl). In some instances, the carboxylic acid group of the C-terminal lysine in [Xaa]_{y} is replaced with —C(O)NH₂.

In some instances, R₃ of Formular (II), (IIa), (IIb), (IIc), or (IId) is C₇₋₁₅ alkenylene. In some instances, R₃ is C₉₋₁₃ alkenylene. In some instances, R₃ is C₁₁ alkenylene. In some instances, R₃ is —(CH₂)₃₋₇—CH═CH—(CH₂)₃₋₇—. In some instances, R₃ is —(CH₂)₅₋₇—CH═CH—(CH₂)₃₋₄—. In some instances, R₃ is —(CH₂)₆—CH═CH—(CH₂)₃—.

In some instances, disclosed herein is a structurally stabilized peptide conjugate comprising or consisting of Formula III, wherein Formula III is defined by:

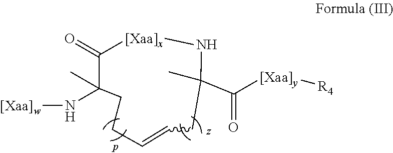

or a pharmaceutically acceptable salt thereof. In some instances, R₄ is **—C(O)—(C₂₋₆ alkylene)-[O—CH₂CH₂]ₘ—N(R₅)C(O)—(C₁₋₆ alkylene)-R₆, wherein ** is the point of attachment to the amino group in the side chain of the C-terminal lysine in [Xaa]_{y}. In some instances, R₅ is hydrogen or C₁₋₄ alkyl. In some instances, R₆ is one of the following:

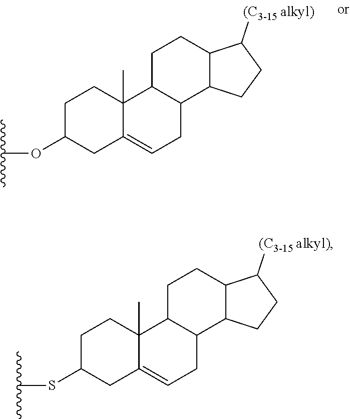

optionally wherein each of which is substituted by t occurrences of R₇.

In some instances, R₇ represents independently for each occurrence C₁₋₃ alkyl, hydroxyl, or C₁₋₃ alkoxyl. In some instances, m is 3, 4, 5, 6, 7, 8, 9, 10, 11, 12, 13, 14, 15, or 16. In some instances, p is 2, 3, 4, 5, 6, 7, or 8. In some instances, z is 2, 3, 4, 5, or 6. In some instances, t is 0, 1, 2, or 3. In some instances, the amino group of the N-terminal aspartic acid in [Xaa]W is replaced with —N(H)C(O)—(C₁₋₄ alkyl). In some instances, the carboxylic acid group of the C-terminal lysine in [Xaa]_{y} is replaced with —C(O)NH₂.

In some instances, the structurally stabilized peptide comprises or consists of Formula (IIIa), which is defined by:

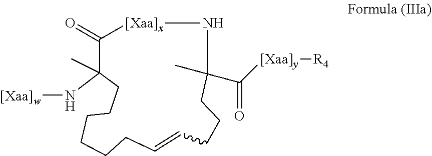

or a pharmaceutically acceptable salt thereof.

In some instances, the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂. In some instances, R₄ is **—C(O)—(C₂₋₃ alkylene)-[O—CH₂CH₂]ₘ—N(R₅)C(O)—(C₁₋₂ alkylene)-R₆, wherein ** is the point of attachment to the amino group in the side chain of the C-terminal lysine in [Xaa]_{y}. In some instances, R₄ is **—C(O)—(CH₂CH₂)—[O—CH₂CH₂]ₘ—N(R₅)C(O)—(CH₂)—R₆, wherein ** is the point of attachment to the amino group in the side chain of the C-terminal lysine in [Xaa]_{y}. In some instances, the amino group of the N-terminal aspartic acid in [Xaa]_{w} is replaced with —N(H)C(O)—(C₁₋₄ alkyl). In some instances, the amino group of the N-terminal aspartic acid in [Xaa]_{w} is replaced with —N(H)C(O)CH₃. In some instances, the carboxylic acid group of the C-terminal lysine in [Xaa]_{y} is replaced with —C(O)NH₂.

In another aspect, disclosed herein is a structurally stabilized peptide conjugate represented by Formula (IV):

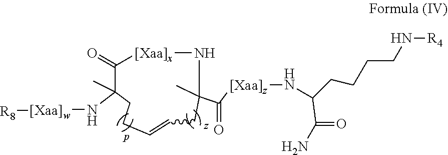

or a pharmaceutically acceptable salt thereof.

In some instances, R₄ is —C(O)—(C₂₋₆ alkylene)-[O—CH₂CH₂]ₘ—N(R₅)C(O)—(C₁₋₆ alkylene)-R₆. In some instances, R₅ is hydrogen or C₁₋₄ alkyl. In some instances, R⁶ is one of the following:

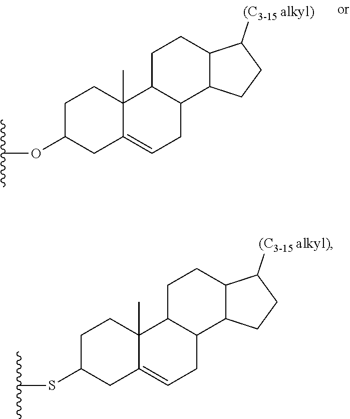

optionally wherein each of which is substituted by t occurrences of R₇.

In some instances, R₇ represents independently for each occurrence C₁₋₃ alkyl, hydroxyl, or C₁₋₃ alkoxyl. In some instances, R₈ is —C(O)—(C₁₋₄ alkyl). In some instances, m is 3, 4, 5, 6, 7, 8, 9, 10, 11, 12, 13, 14, 15, or 16. In some instances, p is 2, 3, 4, 5, 6, 7, or 8. In some instances, z is 2, 3, 4, 5, or 6. In some instances, t is 0, 1, 2, or 3.

In some instances, the structurally stabilized peptide is represented by Formula IVa or a pharmaceutically acceptable salt thereof, where Formula IVa is defined by:

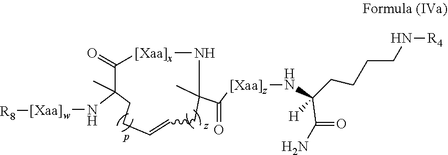

In some instances, the structurally stabilized peptide is represented by Formula IVb or a pharmaceutically acceptable salt thereof, where Formula IVb is defined by:

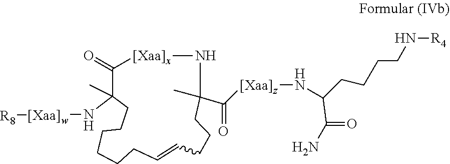

In some instances, the structurally stabilized peptide is represented by Formula IVc or a pharmaceutically acceptable salt thereof, where Formula IVc is defined by:

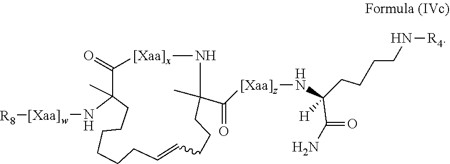

In some instances, disclosed herein is a structurally stabilized peptide conjugate represented by Formula V or a pharmaceutically acceptable salt thereof, wherein Formula V is defined by:

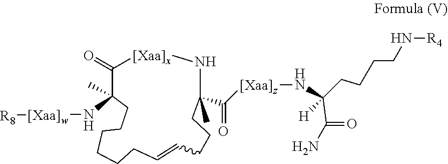

In some instances, R₄ is —C(O)—(C₂₋₆ alkylene)-[O—CH₂CH₂]ₘ—N(R₅)C(O)—(C₁₋₆ alkylene)-R₆. In some instances, R₅ is hydrogen or C₁₋₄ alkyl. In some instances, R⁶ is one of the following:

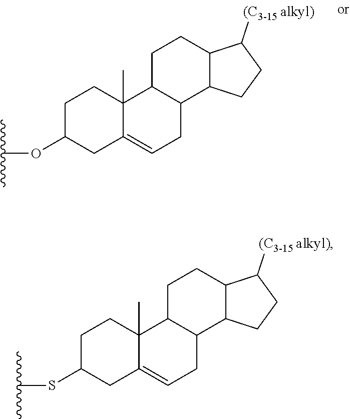

optionally wherein each of which is substituted by t occurrences of R₇.

In some instances, R₇ represents independently for each occurrence C₁₋₃ alkyl, hydroxyl, or C₁₋₃ alkoxyl. In some instances, R₈ is —C(O)—(C₁₋₄ alkyl). In some instances, [Xaa]_{w} is DISGINASVVNIQ (SEQ ID NO: 35); [Xaa]ₓ is EIDRLN (SEQ ID NO: 36); and [Xaa]_{z} is VAKNLNESLIDLQELG (SEQ ID NO: 77). In some instances, m is 3, 4, 5, 6, 7, 8, 9, 10, 11, 12, 13, 14, 15, or 16. In some instances, t is 0, 1, 2, or 3.

In some instances, R₈ is —C(O)CH₃. In some instances, R₄ is —C(O)—(C₂₋₃ alkylene)-[O—CH₂CH₂]ₘ—N(R₅)C(O)—(C₁₋₂ alkylene)-R₆. In some instances, R₄ is —C(O)—(CH₂CH₂)—[O—CH₂CH₂]ₘ—N(R₅)C(O)—(CH₂)—R₆. In some instances, R₅ is (C₃₋₁₅ alkyl) hydrogen. In some instances, R₆ is

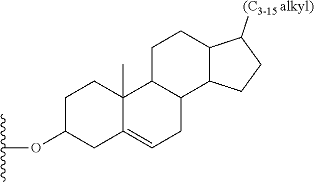

substituted by t occurrences of R₇.

In some instances, R₆ is

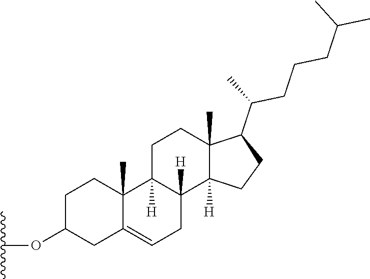

substituted by t occurrences of R₇.

In some instances, R₆ is

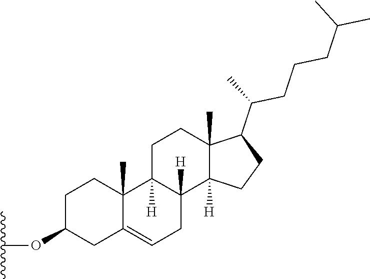

substituted by t occurrences of R₇.

In some instances, R₆ is

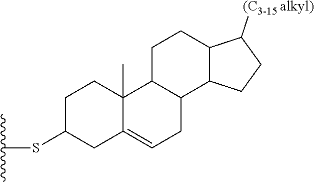

substituted by t occurrences of R₇.

In some instances, R₆ is

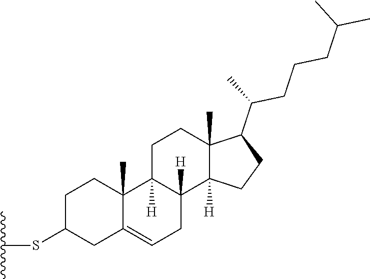

substituted by t occurrences of R₇.

In some instances, R₆ is

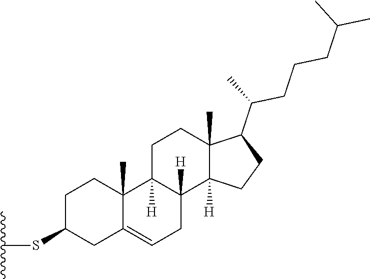

substituted by t occurrences of R₇.

In some instances, wherein t is 0. In some instances, m is 4. In some instances, m is 8.

In another aspect, disclosed herein is a structurally stabilized peptide conjugate represented by Formula VI or a pharmaceutically acceptable salt thereof, wherein Formula VI is defined by:

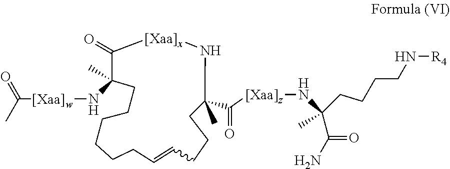

In some instances, R₄ is —C(O)—(CH₂CH₂)—[O—CH₂CH_{2]8}—N(H)C(O)—(CH₂)—R₆. In some instances, R₆ is

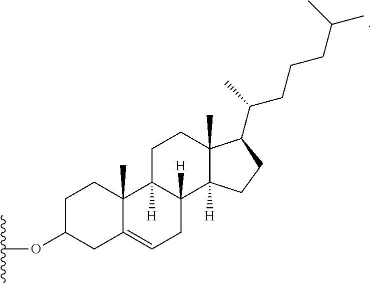

In some instances, [Xaa]_{w} is DISGINASVVNIQ (SEQ ID NO: 35); [Xaa]ₓ is EIDRLN (SEQ ID NO: 36); and [Xaa]_{z} is VAKNLNESLIDLQELG (SEQ ID NO: 77).

In some embodiments, each of the [Xaa]_{w} of Formula (I), the [Xaa]ₓ of Formula (I), and the [Xaa]_{y} of Formulae (I), (Ia), (II), (IIa), is as described for any one of constructs 1-15 of Table 2. For example, for a stabilized peptide comprising the [Xaa]_{w}, the [Xaa]ₓ, and the [Xaa]_{y} of construct 1 of Table 2, the [Xaa]_{w}, the [Xaa]ₓ, and the [Xaa]_{y} is: DISGINASVV (SEQ ID NO: 26), IQKEID (SEQ ID NO: 27), and LNEVAKNLNESLIDLQELGK (SEQ ID NO: 28), respectively. As another example, for a stabilized peptide comprising the [Xaa]_{w}, the [Xaa]ₓ, and the [Xaa]_{y} of construct 2 of Table 2, the [Xaa]_{w}, the [Xaa]ₓ, and the [Xaa]_{y} is: DISGINASVVN (SEQ ID NO: 29), QKEIDR (SEQ ID NO: 30), and NEVAKNLNESLIDLQELGK (SEQ ID NO: 31), respectively.

TABLE 2
[Xaa]_{w}, [Xaa]ₓ, and [Xaa]_{y} sequences for Formula (I)
constructs 1-15.

| Construct | [Xaa]_{w} | [Xaa]ₓ | [Xaa]_{y} |
|---|---|---|---|
| 1 | DISGINASVV (SEQ ID | IQKEID (SEQ ID NO: | LNEVAKNLNESLIDL |
| | NO: 26) | 27) | QELGK (SEQ ID NO: |
| | | | 28) |
| 2 | DISGINASVVN (SEQ | QKEIDR (SEQ ID NO: | NEVAKNLNESLIDLQ |
| | ID NO: 29) | 30) | ELGK (SEQ ID NO: |
| | | | 31) |
| 3 | DISGINASVVNI (SEQ | KEIDRL (SEQ ID NO: | EVAKNLNESLIDLQE |
| | ID NO: 32) | 33) | LGK (SEQ ID NO: 34) |
| 4 | DISGINASVVNIQ | EIDRLN (SEQ ID NO: | VAKNLNESLIDLQEL |
| | (SEQ ID NO: 35) | 36) | GK (SEQ ID NO: 37) |
| 5 | DISGINASVVNIQK | IDRLNE (SEQ ID NO: | AKNLNESLIDLQELG |
| | (SEQ ID NO: 38) | 39) | K (SEQ ID NO: 40) |
| 6 | DISGINASVVNIQKE | DRLNEV (SEQ ID | KNLNESLIDLQELGK |
| | (SEQ ID NO: 41) | NO: 42) | (SEQ ID NO: 43) |
| 7 | DISGINASVVNIQKEI | RLNEVA (SEQ ID | NLNESLIDLQELGK |
| | (SEQ ID NO: 44) | NO: 45) | (SEQ ID NO: 46) |
| 8 | DISGINASVVNIQKEID | LNEVAK (SEQ ID | LNESLIDLQELGK |
| | (SEQ ID NO: 47) | NO: 48) | (SEQ ID NO: 49) |
| 9 | DISGINASVVNIQKEID | NEVAKN (SEQ ID | NESLIDLQELGK |
| | R (SEQ ID NO: 50) | NO: 51) | (SEQ ID NO: 52) |
| 10 | DISGINASVVNIQKEID | EVAKNL (SEQ ID | ESLIDLQELGK (SEQ |
| | RL (SEQ ID NO: 53) | NO: 54) | ID NO: 55) |
| 11 | DISGINASVVNIQKEID | VAKNLN (SEQ ID | SLIDLQELGK (SEQ |
| | RLN (SEQ ID NO: 56) | NO: 57) | ID NO: 58) |
| 12 | DISGINASVVNIQKEID | AKNLNE (SEQ ID | LIDLQELGK (SEQ ID |
| | RLNE (SEQ ID NO: 59) | NO: 60) | NO: 61) |
| 13 | DISGINASVVNIQKEID | KNLNES (SEQ ID | IDLQELGK (SEQ ID |
| | RLNEV (SEQ ID NO: | NO: 63) | NO: 64) |
| | 62 | | |
| 14 | DISGINASVVNIQKEID | NLNESL (SEQ ID NO: | DLQELGK (SEQ ID |
| | RLNEVA (SEQ ID NO: | 66) | NO: 67) |
| | 65) | | |
| 15 | DISGINASVVNIQKEID | LNESLI (SEQ ID NO: | LQELGK (SEQ ID NO: |
| | RLNEVAK (SEQ ID | 69) | 70) |
| | NO: 68) | | |

In certain instances, the sequences set forth above in Table 2 can have at least one (e.g., 1, 2, 3, 4, 5, or 6) amino acid substitution or deletion. The SARS-CoV-2 HR2 peptides can include any amino acid sequence described herein.

In some instances, Formula (I) comprising the sequences set forth above in Table 2 can have one or more of the properties listed below: (i) binds the recombinant SARS-CoV-2 5-helix bundle S protein and/or the corresponding native fusion apparatus; (ii) is alpha-helical; (iii) is protease resistant; (iv) inhibits fusion of SARS-CoV-2 with a host cell; and/or (v) inhibits infection of a cell by SARS-CoV-2. In some instances, the compound binds to a polypeptide comprising or consisting of the sequence of SEQ ID NO:78.

The tether of Formula (I) can include an alkyl, alkenyl, or alkynyl moiety (e.g., C₅, C₈, C₁₁, or C₁₂alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (I), x is 2, 3, or 6. In some instances of Formula (I), each y is independently an integer between 0 and 15, or 3 and 15. In some instances of Formula (I), R₁ and R₂ are each independently H or C₁-C₆ alkyl. In some instances of Formula (I), R₁ and R₂ are each independently C₁-C₃ alkyl. In some instances or Formula (I), at least one of R₁ and R₂ are methyl. For example, R₁ and R₂ can both be methyl. In some instances of Formula (I), R₃ is alkyl (e.g., C₈ alkyl) and x is 3. In some instances of Formula (I), R₃ is C₁₁ alkyl and x is 6. In some instances of Formula (I), R₃ is alkenyl (e.g., C₈ alkenyl) and x is 3. In some instances of Formula (I), x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂—.

In one aspect, a structurally-stabilized COVID-19 HR2 peptide comprises Formula (I), or a pharmaceutically acceptable salt thereof, wherein:
each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
z is 1, 2, 3, 4, 5, 6, 7, 8, 9, or 10; and

(a)
each [Xaa]_{w} is
(SEQ ID NO: 26)
DISGINASVV,
each [Xaa]ₓ is
(SEQ ID NO: 27)
IQKEID,
and
each [Xaa]_{y} is
(SEQ ID NO: 28)
LNEVAKNLNESLIDLQELGK;
(b)
each [Xaa]_{w} is
(SEQ ID NO: 29)
DISGINASVVN,
each [Xaa]ₓ is
(SEQ ID NO: 30)
QKEIDR,
and
each [Xaa]_{y} is
(SEQ ID NO: 31)
NEVAKNLNESLIDLQELGK;
(c)
each [Xaa]_{w} is
(SEQ ID NO: 32)
DISGINASVVNI,
each [Xaa]ₓ is
(SEQ ID NO: 33)
KEIDRL,
and
each [Xaa]_{y} is
(SEQ ID NO: 34)
EVAKNLNESLIDLQELGK;
(d)
each [Xaa]_{w} is
(SEQ ID NO: 35)
DISGINASVVNIQ,
each [Xaa]ₓ is
(SEQ ID NO: 36)
EIDRLN,
and
each [Xaa]_{y} is
(SEQ ID NO: 37)
VAKNLNESLIDLQELGK;
(e)
each [Xaa]_{w} is
(SEQ ID NO: 38)
DISGINASVVNIQK,
each [Xaa]ₓ is
(SEQ ID NO: 39)
IDRLNE,
and
each [Xaa]_{y} is
(SEQ ID NO: 40)
AKNLNESLIDLQELGK;
(f)
each [Xaa]_{w} is
(SEQ ID NO: 41)
DISGINASVVNIQKE,
each [Xaa]ₓ is
(SEQ ID NO: 42)
DRLNEV,
and
each [Xaa]_{y} is
(SEQ ID NO: 43)
KNLNESLIDLQELGK;
(g)
each [Xaa]_{w} is
(SEQ ID NO: 44)
DISGINASVVNIQKEI,
each [Xaa]ₓ is
(SEQ ID NO: 45)
RLNEVA,
and
each [Xaa]_{y} is
(SEQ ID NO: 46)
NLNESLIDLQELGK;
(h)
each [Xaa]_{w} is
(SEQ ID NO: 47)
DISGINASVVNIQKEID,
each [Xaa]ₓ is
(SEQ ID NO: 48)
LNEVAK,
and
each [Xaa]_{y} is
(SEQ ID NO: 49)
LNESLIDLQELGK;
(i)
each [Xaa]_{w} is
(SEQ ID NO: 50)
DISGINASVVNIQKEIDR,
each [Xaa]ₓ is
(SEQ ID NO: 51)
NEVAKN,
and
each [Xaa]_{y} is
(SEQ ID NO: 52)
NESLIDLQELGK;
(j)
each [Xaa]_{w} is
(SEQ ID NO: 53)
DISGINASVVNIQKEIDRL,
each [Xaa]ₓ is
(SEQ ID NO: 54)
EVAKNL,
and
each [Xaa]_{y} is
(SEQ ID NO: 55)
ESLIDLQELGK;
(k)
each [Xaa]_{w} is
(SEQ ID NO: 56)
DISGINASVVNIQKEIDRLN,
each [Xaa]ₓ is
(SEQ ID NO: 57)
VAKNLN,
and
each [Xaa]_{y} is
(SEQ ID NO: 58)
SLIDLQELGK;
(l)
each [Xaa]_{w} is
(SEQ ID NO: 59)
DISGINASVVNIQKEIDRLNE,
each [Xaa]ₓ is
(SEQ ID NO: 60)
AKNLNE,
and
each [Xaa]_{y} is
(SEQ ID NO: 61)
LIDLQELGK;
(m)
each [Xaa]_{w} is
(SEQ ID NO: 62)
DISGINASVVNIQKEIDRLNEV,
each [Xaa]ₓ is
(SEQ ID NO: 63)
KNLNES,
and
each [Xaa]_{y} is
(SEQ ID NO: 64)
IDLQELGK;
(n)
each [Xaa]_{w} is
(SEQ ID NO: 65)
DISGINASVVNIQKEIDRLNEVA,
each [Xaa]ₓ is
(SEQ ID NO: 66)
NLNESL,
and
each [Xaa]_{y} is
(SEQ ID NO: 67)
DLQELGK;
(o)
each [Xaa]_{w} is
(SEQ ID NO: 68)
DISGINASVVNIQKEIDRLNEVAK,
each [Xaa]ₓ is
(SEQ ID NO: 69)
LNESLI,
and
each [Xaa]_{y} is
(SEQ ID NO: 70)
LQELGK,

wherein the structurally-stabilized SARS-CoV-2 HR2 peptide binds the recombinant SARS-CoV-2 5-helix bundle S protein and/or the corresponding native fusion apparatus. In some instances, wherein R₁ is an alkyl. In some instances, R₁ is a methyl group. In some instances, R₃ is an alkyl. In some instances, R₃ is a methyl group. In some instances, R₂ is an alkenyl. In some instances, z is 1.

In another aspect of Formula (I), the two alpha, alpha disubstituted stereocenters are both in the R configuration or S configuration (e.g., i, i+4 cross-link), or one stereocenter is R and the other is S (e.g., i, i+7 cross-link). Thus, where Formula (I) is depicted as:

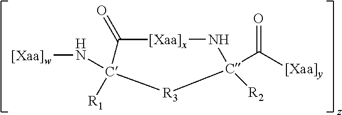

The C′ and C″ disubstituted stereocenters can both be in the R configuration or they can both be in the S configuration. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄—K—R₄]n; and R₄ is a straight chain alkyl, alkenyl, or alkynyl.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like. The term “alkenylene” refers to a linking alkenyl group (e.g., a —CH═CH— or —CH₂CH₂CH═CHCH₂CH₂— group).

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some embodiments, halo is F or Cl.

In some embodiments, the disclosure features structurally-stabilized (e.g., stapled) peptides comprising the amino acid sequence of any one of SEQ ID NOs: 5 or 6 (or a modified version thereof), wherein: the side chains of two amino acids separated by six amino acids are replaced by an internal staple.

The stapled peptide can be 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in length. In a specific embodiment, the stapled peptide is 19-45 amino acids (i.e., 19, 20, 21, 22, 23, 24, 25, 26, 27, 28, 29, 30, 31, 32, 33, 34, 35, 36, 37, 38, 39, 40, 41, 42, 43, 44, 45) in length. In a specific embodiment, the stapled peptide is 36-45 amino acids (i.e., 36, 37, 38, 39, 40, 41, 42, 43, 44, or 45) in length. In a specific embodiment, the stapled peptide is 38-45 amino acids (i.e., 38, 39, 40, 41, 42, 43, 44, or 45) in length. In a specific embodiment, the stapled peptide is 36-42 amino acids (i.e., 36, 37, 38, 39, 40, 41, 42) amino acids in length. In a specific embodiment, the stapled peptide is 38-42 amino acids (i.e., 38, 39, 40, 41, 42) amino acids in length. In a specific embodiment, the stapled peptide is 36 amino acids in length. In another specific embodiment, the stapled peptide is 38 amino acids in length. Exemplary COVID-19 HR2 stapled peptides are shown in Tables 1 and 2 and described in Formula (I). In one embodiment, the COVID-19 HR2 stapled peptide comprises or consists of a stapled version of the amino acid sequence of any one of SEQ ID NOs: 7-21 (e.g., the product of a ring-closing metathesis reaction performed on a peptide comprising the amino acid sequence of any one of SEQ ID NOs: 7-21, respectively). In one embodiment, the SARS-CoV-2 HR2 stapled peptide comprises or consists of a stapled version of the amino acid sequence of SEQ ID NO: 5 (e.g., the product of a ring-closing metathesis reaction performed on a peptide comprising the amino acid sequence of SEQ ID NO: 5). In one embodiment, the SARS-CoV-2 HR2 stapled peptide comprises or consists of a stapled version of the amino acid sequence of SEQ ID NO: 6 (e.g., the product of a ring-closing metathesis reaction performed on a peptide comprising the amino acid sequence of SEQ ID NO: 6).

In certain embodiments, the two amino acids each separated by six amino acids are at the amino acid positions in the SARS-CoV-2 HR2 peptide corresponding to positions 11 and 18 of SEQ ID NO:6. In certain embodiments, the two amino acids each separated by six amino acids are at the amino acid positions in the SARS-CoV-2 HR2 peptide corresponding to positions 12 and 19 of SEQ ID NO:6. In certain embodiments, the two amino acids each separated by six amino acids are at the amino acid positions in the SARS-CoV-2 HR2 peptide corresponding to positions 13 and 20 of SEQ ID NO:6. In certain embodiments, the two amino acids each separated by six amino acids are at the amino acid positions in the SARS-CoV-2 HR2 peptide corresponding to positions 14 and 21 of SEQ ID NO:6. In certain embodiments, the two amino acids each separated by six amino acids are at the amino acid positions in the SARS-CoV-2 HR2 peptide corresponding to positions 15 and 22 of SEQ ID NO:6. In certain embodiments, the two amino acids each separated by six amino acids are at the amino acid positions in the SARS-CoV-2 HR2 peptide corresponding to positions 16 and 23 of SEQ ID NO:6. In certain embodiments, the two amino acids each separated by six amino acids are at the amino acid positions in the SARS-CoV-2 HR2 peptide corresponding to positions 17 and 24 of SEQ ID NO:6. In certain embodiments, the two amino acids each separated by six amino acids are at the amino acid positions in the SARS-CoV-2 HR2 peptide corresponding to positions 18 and 25 of SEQ ID NO:6. In certain embodiments, the two amino acids each separated by six amino acids are at the amino acid positions in the SARS-CoV-2 HR2 peptide corresponding to positions 19 and 26 of SEQ ID NO:6. In certain embodiments, the two amino acids each separated by six amino acids are at the amino acid positions in the SARS-CoV-2 HR2 peptide corresponding to positions 20 and 27 of SEQ ID NO:6. In certain embodiments, the two amino acids each separated by six amino acids are at the amino acid positions in the SARS-CoV-2 HR2 peptide corresponding to positions 21 and 28 of SEQ ID NO:6. In certain embodiments, the two amino acids each separated by six amino acids are at the amino acid positions in the SARS-CoV-2 HR2 peptide corresponding to positions 22 and 29 of SEQ ID NO:6. In certain embodiments, the two amino acids each separated by six amino acids are at the amino acid positions in the SARS-CoV-2 HR2 peptide corresponding to positions 23 and 30 of SEQ ID NO:6. In certain embodiments, the two amino acids each separated by six amino acids are at the amino acid positions in the SARS-CoV-2 HR2 peptide corresponding to positions 24 and 31 of SEQ ID NO:6. In certain embodiments, the two amino acids each separated by six amino acids are at the amino acid positions in the SARS-CoV-2 HR2 peptide corresponding to positions 25 and 32 of SEQ ID NO:6.

While hydrocarbon tethers are provided herein, other tethers can also be employed in the structurally-stabilized SARS-CoV-2 HR2 peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the SARS-CoV-2 HR2 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A., 97:3803-3808 (2000); Double-click stapling: Lau et al., Chem. Sci., 5:1804-1809 (2014); Bis-lactam stapling: J. C. Phelan et al., J Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+7 are provided herein in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

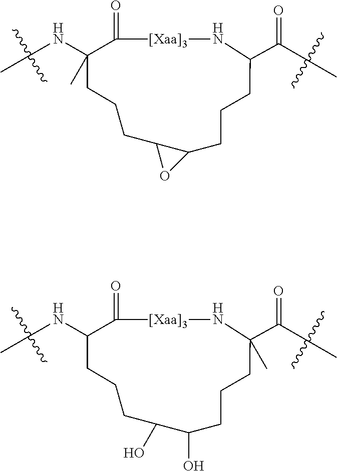

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, alpha disubstituted amino acids are used in the peptide to improve the stability of the alpha helical secondary structure. However, alpha disubstituted amino acids are not required, and instances using mono-alpha substituents (e.g., in the tethered amino acids) are also envisioned.

The structurally-stabilized (e.g., stapled) peptides can include a drug, a toxin, a derivative of polyethylene glycol; a second peptide; a carbohydrate, etc. Where a polymer or other agent is linked to the structurally-stabilized (e.g., stapled) peptide, it can be desirable for the composition to be substantially homogeneous.

The addition of polyethelene glycol (PEG) molecules can improve the pharmacokinetic and pharmacodynamic properties of the peptide. For example, PEGylation can reduce renal clearance and can result in a more stable plasma concentration. PEG is a water soluble polymer and can be represented as linked to the peptide as formula:

XO—(CH₂CH₂O)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG as used herein in some instances functions as a linker or spacer between one of the peptides (e.g., stapled peptides; e.g., SEQ ID NO:7-21) and a cholesterol or thiocholesterol moiety. In some instances, the PEG molecule comprises the following formula, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

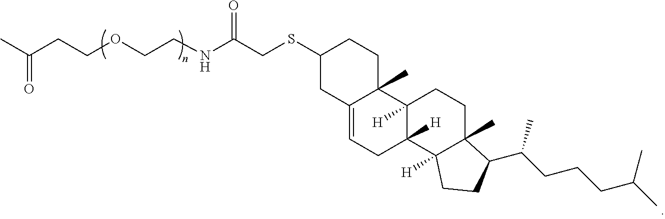

In some instances, the PEG molecule comprises the following formula, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

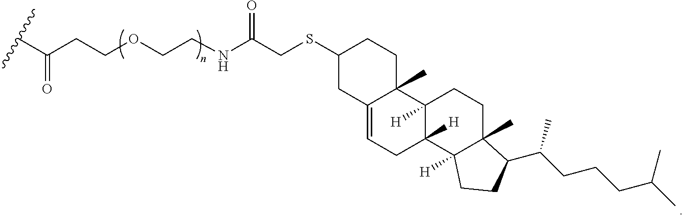

In some instances, the PEG molecule comprises the following formula, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

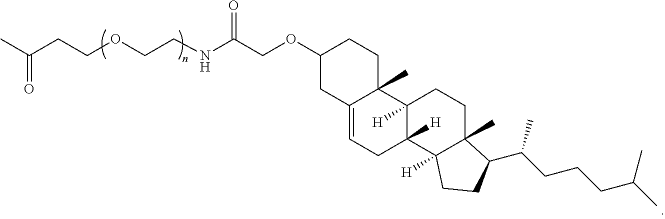

In some instances, the PEG molecule comprises the following formula, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

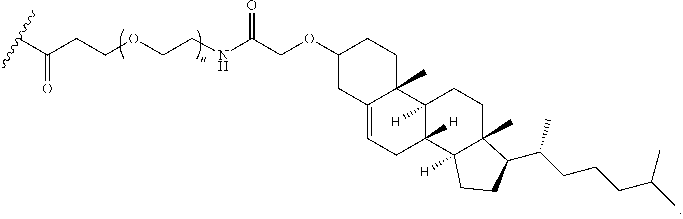

In some instances for each of the formulae above, n=4. In some instances for each of the formulae above, n=5. In some instances for each of the formulae above, n=6. In some instances for each of the formulae above, n=7. In some instances for each of the formulae above, n=8.

In some instances, the PEG molecule includes a cholesterol moiety. In some instances, the cholesterol moiety is thiocholesterol. In some instances, the sulfur of the thioether moiety in thiocholesterol is replaced by an oxygen atom to produce an ether moiety in the cholesterol derivatization.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to a structurally-stabilized (e.g., stapled) peptide described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The structurally-stabilized (e.g., stapled) peptides can also be modified, e.g., to further facilitate mucoadhesion, membrane binding, or increase in vivo stability, in some embodiments. For example, acylating or PEGylating a structurally-stabilized peptide increases bioavailability, increases blood circulation, alters pharmacokinetics, alters immunogenicity and/or decreases the needed frequency of administration.

In some embodiments, the structurally-stabilized (e.g., stapled) peptides disclosed herein have an enhanced ability to bind to or penetrate cell membranes (e.g. relative to non-stabilized peptides). See, e.g., International Publication No. WO 2017/147283, which is incorporated by reference herein in its entirety.

### Methods of Treatment

The disclosure features methods of using any of the structurally-stabilized (e.g., stapled) peptide-cholesterol conjugates (or pharmaceutical compositions comprising said structurally-stabilized peptide-cholesterol conjugates) described herein for the prevention and/or treatment of a coronavirus (e.g., betacoronavirus such as SARS-CoV-2) infection or coronavirus disease (e.g., COVID-19). The terms “treat” or “treating,” as used herein, refers to alleviating, inhibiting, or ameliorating the disease or infection from which the subject (e.g., human) or other species (e.g., pets; farm animals; domestic animals) is suffering. In some instances, the subject is an animal. In some embodiments, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat or other species that spread coronavirus. In some instances, the subject is a human. In certain embodiments, such terms refer to a non-human animal (e.g., a non-human animal such as a pig, horse, cow, cat or dog). In some embodiments, such terms refer to a pet or farm animal. In some embodiments, such terms refer to a human.

The structurally-stabilized (e.g., stapled) peptide-cholesterol conjugates (or compositions comprising the peptides) described herein can be useful for treating a subject (e.g., human subject or a species as described above) having a coronavirus (e.g., betacoronavirus) infection. The structurally-stabilized (e.g., stapled) peptide-cholesterol conjugates (or compositions comprising the peptide-cholesterol conjugates) described herein can also be useful for treating a human subject or another species provided herein having a coronavirus disease. The structurally-stabilized (e.g., stapled) peptide-cholesterol conjugates (or compositions comprising the peptide-cholesterol conjugates) described herein can also be useful for treating a subject having a coronavirus disease, wherein the subject is a mammal such as a non-primate (e.g., cow, pig, horse, cat, dog, rat, etc.) or a primate (e.g., monkey or human).

In certain embodiments, the coronavirus infection is an infection of one of 229E (alpha coronavirus); NL63 (alpha coronavirus); OC43 (beta coronavirus); HKU1 (beta coronavirus); Middle East respiratory syndrome (MERS); SARS-CoV; or SARS-CoV-2. In certain embodiments, the coronavirus infection is an infection of a SARS-CoV-2 variant selected from one of D614G B.1 (RVP-702), Wuhan-Hu-1 QHD43416.1 (RVP-701), New York variant B.1.526 (RVP-726) Iota, Californian variant B.1.526 (RVP-713), or UK variant B.1.1.7 with E484K (RVP-717). In certain embodiments, the coronavirus disease is caused by a COVID-19 infection.

In certain embodiments, the coronavirus infection is an infection of one of Wuhan-Hu-1, B.1.427/B.1.429, B.1.617.2, D614G B.1, Brazilian variant P.1, B.1.1.7, B.1.351, B.1.525, B.1.526, B.1.617.1, B.1.617.3, P.2, B.1.621, B.1.621.1, B.1.1.529, BA.1, BA.1.1, BA.2, BA.3, BA.4 or BA.5. In certain embodiments, the coronavirus infection is an infection of one of B.1.351, Cluster 5, Lineage B.1.1.207, Lineage B.1.1.7, Variant of Concern 202102/02, Lineage B.1.1.317, Lineage B.1.1.318, Lineage B.1.351, Lineage B.1.429, Lineage B.1.525, Lineage P.1 (also known as Lineage B.1.1.28), Lineage B.1.1.529, Lineage BA.1, Lineage BA.1.1, Lineage BA.2, Lineage BA.3, Lineage BA.4 Lineage BA.5, D614G, E484K, N501Y, S477G/N, or P681H.

The structurally-stabilized (e.g., stapled) peptide-cholesterol conjugates (or compositions comprising the peptide-cholesterol conjugates) described herein can be useful for preventing a coronavirus (e.g., betacoronavirus) infection in a human subject or a subject from another species provided herein. The peptide-cholesterol conjugates (or compositions comprising the peptide-cholesterol conjugates) described herein can also be useful for preventing a coronavirus disease in a subject (e.g., human subject) or a subject from another species provided herein. In certain embodiments, the coronavirus infection is an infection of one of 229E (alpha coronavirus); NL63 (alpha coronavirus); OC43 (beta coronavirus); HKU1 (beta coronavirus); Middle East respiratory syndrome (MERS); SARS-CoV; or SARS-COVID-19. In certain embodiments, the coronavirus disease is caused by a COVID-19 infection.

In certain embodiments, the human subject or a subject from another species provided herein in need thereof is administered a peptide described in Table 1 or 2, or a variant thereof. In certain embodiments, the human subject or a subject from another species provided herein in need thereof is administered a stapled SARS-CoV-2 HR2 peptide-cholesterol conjugate comprising or consisting of SEQ ID NO: 6 or a modified version thereof. In certain embodiments, the human subject in need thereof is administered a stapled SARS-CoV-2 HR2 peptide-cholesterol conjugate comprising or consisting of SEQ ID NO: 5 or a modified version thereof.

In certain embodiments, the human subject or a subject from another species provided herein in need thereof is administered any one of the peptide-cholesterol conjugates having SEQ ID NOs: 7-21 described in Table 1, or a variant thereof (as described herein). Possible variations in these peptide-cholesterol conjugates are described in the Structurally Stabilized Peptide section. A variant of these sequences has at least one (e.g., 1, 2, 3, 4, 5) of these properties: (i) is alpha-helical; (ii) is protease resistant; (iii) inhibits fusion of SARS-CoV-2 with a host cell; and/or (iv) inhibits infection of a cell by SARS-CoV-2. In some instances, the structurally stabilized peptides having a variant inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays. In certain embodiments, the treatment is made to block transmission between human subjects. In some instances, the treatment controls the spread of infection in a population of human subjects.

In certain embodiments, the human subject or a subject from another species provided herein in need thereof is administered a peptide having at least 50%, 55%, 60%, 65%, 70%, 75%, 80%, 85%, 90%, 92%, 94%, 95%, identity to any one of the peptide-cholesterol conjugates having SEQ ID NOs: 7-21. In certain embodiments, the human subject or a subject from another species provided herein in need thereof is administered any one of the peptide-cholesterol conjugates having SEQ ID NOs: 7-21 but having 1-10 (e.g., 1, 2, 3, 4, 5, 6, 7, 8, 9, 10) amino acid substitutions, insertions, and/or deletions. In certain embodiments, the human subject or a subject from another species provided herein in need thereof is administered any one of the peptide-cholesterol conjugates having an amino acid sequence comprising any one of SEQ ID NOs: 10, 13, 17, or 20 but having 1-10 (e.g., 1, 2, 3, 4, 5, 6, 7, 8, 9, 10) amino acid substitutions, insertions, and/or deletions.

In some embodiments, the human subject or a subject from another species provided herein is infected with a coronavirus (e.g., betacoronavirus). In some embodiments, the human subject or a subject from another species provided herein is at risk of being infected with a coronavirus (e.g., betacoronavirus). In some embodiments, the human subject or a subject from another species provided herein is at risk of developing a coronavirus disease (e.g., betacoronavirus). In some instances, a human subject or a subject from another species provided herein is at risk of being infected with a coronavirus or at risk of developing a coronavirus disease if he or she or the a subject from another species provided herein lives in an area (e.g., city, state, country) subject to an active coronavirus outbreak (e.g., an area where at least 1, at least 2, at least 3, at least 4, at least 5, at least 6, at least 7, at least 8, at least 9, at least 10, at least 20, at least 30, at least 40, or more people have been diagnosed as infected with a coronavirus). In some instances, a human subject or a subject from another species provided herein is at risk of being infected with a coronavirus or developing a coronavirus disease if he or she or a subject from another species provided herein lives in an area near (e.g., a bordering city, state, country) a second area (e.g., city, state, country) subject to an active coronavirus outbreak (e.g., an area near (e.g., bordering) a second area where at least 1, at least 2, at least 3, at least 4, at least 5, at least 6, at least 7, at least 8, at least 9, at least 10, at least 20, at least 30, at least 40, or more people have been diagnosed as infected with a coronavirus). In certain embodiments, the coronavirus disease is caused by a SARS-CoV-2 infection. In certain embodiments, the subject or a subject from another species provided herein has or is at risk of developing COVID-19.

In general, methods include selecting a subject or a subject from another species provided herein and administering to the subject or a subject from another species provided herein an effective amount of one or more of the structurally-stabilized (e.g., stapled) peptides herein, e.g., in or as a pharmaceutical composition, and optionally repeating administration as required for the prevention or treatment of a coronavirus infection or a coronavirus disease and can be administered orally, intranasally, intravenously, intradermally, subcutaneously, intramuscularly, or topically, including skin, nasal, sinus, ocular, oropharynx, respiratory tree, and lung administration. In some instances, the administration is by a topical respiratory application which includes application to the nasal mucosa, sinus mucosa, oropharyngeal mucosa, or respiratory tree, including the lungs. In some instances, topical application includes application to the skin or eyes. A subject can be selected for treatment based on, e.g., determining that the subject is at risk to acquire or has a coronavirus (e.g., betacoronavirus such as SARS-CoV-2) infection. The peptide-cholesterol conjugates of this disclosure can be used to determine if a subject is infected with a coronavirus. In some instances, the peptide-cholesterol conjugates described herein increase bioavailability, increase blood circulation, alter pharmacokinetics, decrease immunogenicity and/or decrease the needed frequency of administration.

Specific dosage and treatment regimens for any particular patient or subject will depend upon a variety of factors, including the activity of the specific compound employed, the age, body weight, general health status, sex, diet, time of administration, rate of excretion, drug combination, the severity and course of the disease, condition or symptoms, the patient's or subject's disposition to the disease, condition or symptoms, and the judgment of the treating physician or veterinarian.

An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week, including once every other day. The skilled artisan will appreciate that certain factors may influence the dosage and timing required to effectively treat a subject, including but not limited to the risk to acquire or severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Pharmaceutical Compositions

One or more of any of the structurally-stabilized (e.g., stapled) peptide-cholesterol conjugates described herein can be formulated for use as or in pharmaceutical compositions. The pharmaceutical compositions may be used in the methods of treatment or prevention described herein (see above). In certain embodiments, the pharmaceutical composition comprises a structurally-stabilized (e.g., stapled) peptide-cholesterol conjugate comprising or consisting of an amino acid sequence that is identical to an amino acid sequence set forth in Table 1, except for 1 to 10, 1 to 9, 1 to 8, 1 to 7, 1 to 6, 1 to 5, 1 to 4, 1 to 3, 1 to 2, or 1 amino acid substitution, insertion, or deletion. These changes to the amino acid sequences can be made on the non-interacting alpha-helical face of these peptides (i.e., to the amino acids that do not interact with the coronavirus 6-helix bundle fusion apparatus) and/or on the interacting alpha-helical face (i.e., to the amino acids that interact with the coronavirus 6-helix bundle fusion apparatus). Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays, eye drops, and/or solutions).

In some instances, pharmaceutical compositions can include an effective amount of one or more structurally-stabilized (e.g., stapled) peptide-cholesterol conjugates. The terms “effective amount” and “effective to treat,” as used herein, refer to an amount or a concentration of one or more structurally-stabilized (e.g. stapled) peptide-cholesterol conjugates or a pharmaceutical composition described herein utilized for a period of time (including acute or chronic administration and periodic or continuous administration) that is effective within the context of its administration for causing an intended effect or physiological outcome (e.g., treatment of infection).

Pharmaceutical compositions of this invention can include one or more structurally-stabilized (e.g., stapled) peptide-cholesterol conjugates described herein and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient or a subject from another species provided herein, together with a compound of this invention, and which does not destroy the pharmacological activity thereof and is nontoxic when administered in doses sufficient to deliver a therapeutic amount of the compound.

In some instances, the pharmaceutical compositions of this disclosure include one or more of acetate, citrate and/or maleate. In some instances, the pharmaceutical compositions can include water or phosphate buffer saline (PBS). In some instance, the pharmaceutical compositions can include chitosan.

The pharmaceutical compositions disclosed herein can include one or more pharmaceutically acceptable salts. In some instances, the pharmaceutically acceptable salts include salts comprising hydrochloride, sodium, sulfate, acetate, phosphate or diphosphate, chloride, potassium, maleate, calcium, citrate, mesylate, nitrate, tartrate, aluminum, gluconate, and any combination thereof.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled) peptide-cholesterol conjugates disclosed herein can be further conjugated, for example, to a carrier protein. Such conjugated compositions can be monovalent or multivalent. For example, conjugated compositions can include one structurally-stabilized (e.g. stapled) peptide-cholesterol conjugate disclosed herein conjugated to a carrier protein. Alternatively, conjugated compositions can include two or more structurally-stabilized (e.g., stapled) peptide-cholesterol conjugates disclosed herein further conjugated to a carrier.

As used herein, when two entities are “conjugated” to one another they are linked by a direct or indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, electrostatic interactions, etc. An indirect covalent interaction occurs when two entities are covalently connected, optionally through a linker group.

Carrier proteins can include any protein that increases or enhances stability, half-life, tissue exposure, and/or immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561, 1991; Szu et al., J Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

Methods of Making Stapled or Stitched Peptides Derivatized with PEG(n)-Thiocholesterol or PEG(n)-Cholesterol Moieties

In one aspect this disclosure features a method of making a structurally-stabilized peptide derivatized with PEG(n)-thiocholesterol or PEG(n)-cholesterol Moieties. The fully on-resin synthetic method involves (a) providing a peptide comprising at least two non-natural amino acids with olefinic side chains (e.g., SEQ ID NO: 7-21), (b) cross-linking the peptide, in some instances by a ruthenium catalyzed metathesis reaction, and (c) derivatizing the C-terminus on resin with a PEG linker of variable length connected to a thiocholesterol or cholesterol moiety.

In some instances, the methods include cleaving the structurally-stabilized peptide from the resin. Cleaving a structurally-stabilized resin is known in the art. In some instances, cleaving occurs prior to derivatizing the C-terminus on resin with a PEG linker of variable length connected to a thiocholesterol or cholesterol moiety. See e.g., de Vries et al., Science, 2021 Mar. 26; 371(6536):1379-138, and Figueira et al., J. Virol. 91, e01554-16 (2016), each of which is incorporated by reference in its entirety. In other aspects, cleaving is performed after the step of derivatizing the C-terminus on resin with a PEG linker of variable length connected to a thiocholesterol or cholesterol moiety.

In instances in which the cleaving step is performed prior to the derivatizing step, the methods include use of a compound having one of the following formulae:

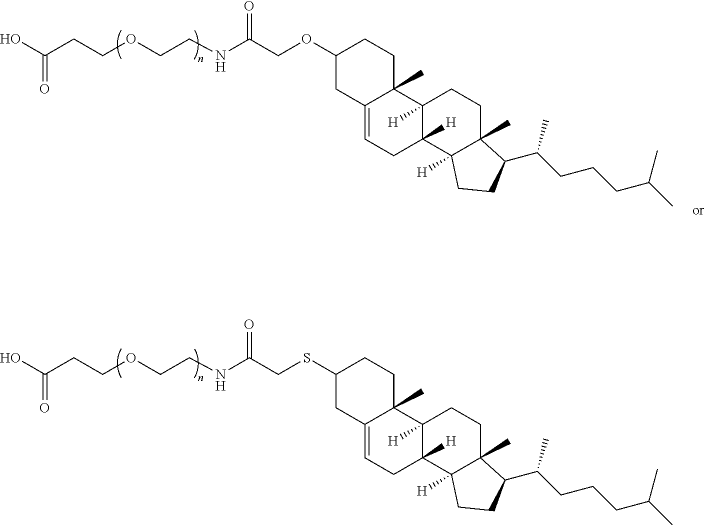

wherein n is 1-36. In some instances, n is 4, 5, 6, 7, 8, 10, 12, 14, 16, 18, 20, 22, 24, 26, 28, 30, 32, 34, or 36. Thus, also disclosed herein are compounds having one of the above formulae.

In some aspects, also disclosed herein is a method for synthesizing a stabilized peptide (i.e., any of the peptides disclosed herein; e.g., SEQ ID NO:10), comprising the step of subjecting a peptide comprising Formula VII to ring closing metathesis conditions to provide a stabilized peptide, wherein Formula VII is defined by:

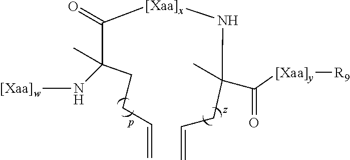

or a pharmaceutically acceptable salt thereof. In some instances, [Xaa]_{w} is DISGINASVVNIQ (SEQ ID NO: 35); [Xaa]ₓ is EIDRLN (SEQ ID NO: 36); and [Xaa]_{y} is VAKNLNESLIDLQELGK (SEQ ID NO: 37). In some instances, [Xaa]_{w}, [Xaa]ₓ, and [Xaa]_{y} are disclosed in Table 2. In some instances, R₉ is —OH or

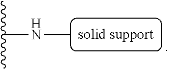 [SMILES: *NC]

In some instances, p is 2, 3, 4, 5, 6, 7, or 8. In some instances, z is 2, 3, 4, 5, or 6. In some instances, the amino group of the N-terminal aspartic acid in [Xaa]W is replaced with —N(H)C(O)—(C₁₋₄ alkyl). In some instances, the ring closing metathesis conditions comprise exposing said peptide comprising Formula IV to a Grubbs ring closing metathesis ruthenium catalyst.
In some instances, the stabilized peptide comprises Formula III, which is defined by:

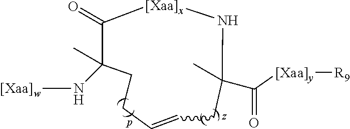

or a pharmaceutically acceptable salt thereof, wherein:

[Xaa]_{w} is
(SEQ ID NO: 35)
DISGINASVVNIQ;
[Xaa]ₓ is
(SEQ ID NO: 36)
EIDRLN;
[Xaa]_{y} is
(SEQ ID NO: 37)
VAKNLNESLIDLQELGK;

R₉ is —OH or

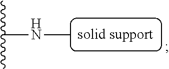 [SMILES: *NC]

p is 2, 3, 4, 5, 6, 7, or 8; an
z is 2, 3, 4, 5, or 6; wherein optionally the amino group of the N-terminal aspartic acid in [Xaa]_{w} is replaced with —N(H)C(O)—(C₁₋₄ alkyl). In some instances, p is 4. In some instances, p is 5. In some instances, p is 6. In some instances, z is 3. In some instances, the methods include derivatizing the C-terminus end of the stabilized peptide with a moiety comprising a polyethylene glycol moiety and a cholesterol or thiocholesterol moiety. In some instances, the methods further include derivatizing the C-terminus end of the stabilized peptide to install a **—C(O)—(C₂₋₆ alkylene)-[O—CH₂CH₂]ₘ—N(R₅)C(O)—(C₁₋₆ alkylene)-R₆ group, wherein ** is the point of attachment to the amino group in the side chain of the C-terminal lysine in [Xaa]_{y}. In some instances, R₅ is hydrogen or C₁₋₄ alkyl. In some instances, R₆ is one of the following:

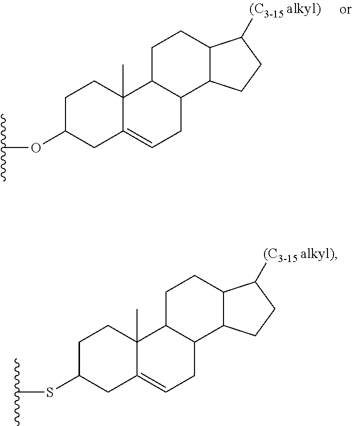

m is 3, 4, 5, 6, 7, 8, 9, 10, 11, 12, 13, 14, 15, or 16; and
t is 0, 1, 2, or 3. In some instances, m is 4. In some instances, m is 8. In some instances, R₉ is

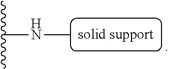 [SMILES: *NC]

In some instances, the peptide is cleaved.

In some instances, the above methods further include formulating the stabilized peptide or salt thereof into a sterile pharmaceutical composition.

Stapled peptide synthesis: Fmoc-based solid-phase peptide synthesis was used to synthesize stapled peptide fusion inhibitors in accordance with our reported methods for generating all-hydrocarbon stapled peptides (Bird et al., Curr. Protocol. Chem, Biol., 3(3):99-117 (2011; Bird et al., Methods Enzymol., 446:369-86(2008). To achieve the various staple lengths, α-methyl, α-alkenyl amino acids were installed in specific pairings at discrete positions, such as for i, i+7 positioning the use of one S-pentenyl alanine residue (S5) and one R-octenyl alanine residue (R₈). For the stapling reaction, Grubbs 1st generation ruthenium catalyst dissolved in dichloroethane was added to the resin-bound peptides. To ensure maximal conversion, three to five rounds of stapling were performed. After appending the PEG(n)-thiocholesterol or PEG(n)-cholesterol moiety (see below), the peptides were then cleaved off of the resin using trifluoroacetic acid, precipitated using a hexane:ether (1:1) mixture, air dried, and purified by LC-MS. All peptides were quantified by amino acid analysis.

Stitched peptide synthesis: Methods of synthesizing the stitched peptides described herein are known in the art. Nevertheless, the following exemplary method may be used. Synthetic chemistry transformations and protecting group methodologies (protection and deprotection) useful in synthesizing the compounds described herein are known in the art and include, for example, those such as described in Bird et al., ACS Chem Biol. (2020) 15(6):1340-1348; Hilinski et al., J Am Chem Soc. (2014) 136(35):12314-22; R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

C-terminal derivatization of stapled or stitched peptides with PEG(n)-thiocholesterol or PEG(n)-cholesterol using an on-resin synthetic approach: To generate the carboxy thiocholesterol or carboxy cholesterol reagent for peptide derivatization by solid phase synthesis, thiocholesterol was dissolved in dichloromethane (DCM) or cholesterol was dissolved in tetrahydrofuran (THF) at 0.1 M and added to a round bottom flask. 3 eq of a base (diisopropylethylamine for thiocholesterol or sodium hydride or potassium t-butoxide for cholesterol) was added with stirring. 5 eq of the t-butyl ester of bromoacetic acid was added next and the reaction was stirred for 2 hours at room temperature followed by 30 min at 40° C. Two volumes of trifluoroacetic acid (relative to solvent) was added and the reaction was stirred at room temperature for 30 min. The reaction progress was monitored by TLC (19:1 Hex:EtOAc for thiocholesterol and 3:1 Hex:EtOAc for cholesterol) with KMnO₄ staining. (Thio)cholesterol, for example, migrated with the solvent front with (thio)ether slowing migration by ˜20% and TFA hydrolysis brought the spot to baseline. The reaction mixture was added to 5 vol water and the 1 vol DCM was added. The solvent layer was washed with 0.1M HCl, brine and dried with sodium sulfate. Removal of the solvent by Rotovap yielded an orange heavy oil that was used without further purification. The yield was near quantitative. Purity was determined to be greater than 90% by NMR of the olefin proton vs the new CH₂ singlet. For peptide derivatization with thiocholesterol or cholesterol, the completed resin bound peptide sequence was treated with 20% piperidine/DMF followed by capping with acetic anhydride to block the N-terminal amine before the C-terminal side chain lysine amine was revealed by treatment with 2% hydrazine in DMF, 5× for 10 min each. The amine was acylated with an Fmoc-protected PEG(n) amino acid (e.g., n=1-36 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36)) at which point the olefins were crosslinked by treating with Grubbs(I) catalyst, 3× for 2 h each. Upon completion, the Fmoc was removed from the C-terminal NH of the PEG reagent and the amine was acylated with carboxy-thiocholesterol (or carboxy-cholesterol) for 30 min. TFA cleavage yielded a crude product of excellent purity that was further purified using semi-prep HPLC.

The peptide sequences of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Insertion of a linking amino acid may be performed as described in, e.g., Young and Schultz, J Biol Chem. 2010 Apr. 9; 285(15): 11039-11044. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimal for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from, e.g., Advanced Chemtech or Gyros Protein Technologies. Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (0-CH₂ or CH₂-0); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The peptides can be further modified by: acetylation, amidation, biotinylation, cinnamoylation, farnesylation, fluoresceination, formylation, myristoylation, palmitoylation, and other lipidation, specifically including thiocholesterol or cholesterol modification using the on-resin method disclosed herein, phosphorylation (Ser, Tyr or Thr), stearoylation, succinylation and sulfurylation. As indicated above, peptides can be conjugated to or contain linker atoms or moieities of variable length, for example, polyethylene glycol (PEG) moieties of variable length; alkyl groups (e.g., C1-C20 straight or branched alkyl groups); fatty acid radicals; and combinations thereof α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). In some instances the stitched peptide comprises a kinkage between i, i+4, and i+4 and i+8. Such stitched peptides can be made in the context of SEQ ID NO:4 or SEQ ID NO:6. In some instances, the amino acids forming the staple or stitch are (R)-2-(4′-pentenyl)Alanine, 2,2-bis(4-pentenyl)glycine, and (S)-2-(4′-pentenyl)Alanine at positions i, i+4, and i+8, respectively, of the stitch. In some instances for peptides where an i linked to i+7, i+7 linked to i+14 stitch is used (four turns of the helix stabilized): one R-octenyl alanine (e.g., (R)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one S-octenyl alanine (e.g., (S)-α-(7′-octenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. R-octenyl alanine is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin or Rink Amide AM resin (see, e.g., WO 2010/148335).

Fmoc-protected α-amino acids (other than the olefinic amino acids N-Fmoc-α,α-Bis(4′-pentenyl)glycine, (S)—N-Fmoc-α-(4′-pentenyl)alanine, (R)—N-Fmoc-α-(7′-octenyl)alanine, (R)—N-Fmoc-α-(7′-octenyl)alanine, and (R)—N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stitching, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some instances, the peptides are substantially free of non-stitched or non-stapled peptide contaminants or are isolated. Methods for purifying peptides include, for example, synthesizing the peptide on a solid-phase support. Following cyclization, multiple alternative solvent and purification schemes are known in the art for peptide and stapled peptide isolation and purification and may use solvents that include, but are not limited to, DMSO, DMSO/dichloromethane mixture, DMSO/NMP mixture, or a mixture/solution that does not include DMSO. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Properties of the stitched or stapled peptides derivatized with a C-terminal PEG(n)-thiocholesterol or PEG(n)-cholesterol of the disclosure can be assayed, for example, using the methods described below and in the Examples.

Assays to Determine Characteristics and Anti-SARS-CoV-2 Activity of Stapled HR2 Peptides Derivatized with PEG(n)-Thiocholesterol or PEG(n)-Cholesterol Moieties

Assays to Determine α-Helicity: Compounds are dissolved in an aqueous solution (e.g. 5 μM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol., 1986).

Assays to Determine Melting Temperature (Tm): Cross-linked or the unmodified template peptides are dissolved in distilled H₂O or other buffer or solvent (e.g. at a final concentration of 50 μM) and Tm is determined by measuring the change in ellipticity over a temperature range (e.g. 4 to 95° C.) on a spectropolarimeter (e.g., Jasco J-710, Aviv) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In Vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g. trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N2<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

Plasma Stability Assay: Stapled peptide stability can be tested in freshly drawn mouse plasma collected in lithium heparin tubes. Triplicate incubations are set up with 500 μl of plasma spiked with 10 μM of the individual peptides. Samples are gently shaken in an orbital shaker at 37° C. and 25 μl aliquots are removed at 0, 5, 15, 30, 60, 240, 360 and 480 min and added to 100 μl of a mixture containing 10% methanol:10% water:80% acetonitrile to stop further degradation of the peptides. The samples are allowed to sit on ice for the duration of the assay and then transferred to a MultiScreen Solvinert 0.45 μm low-binding hydrophilic PTFE plate (Millipore). The filtrate is directly analyzed by LC-MS/MS. The peptides are detected as double or triple charged ions using a Sciex 5500 mass spectrometer. The percentage of remaining peptide is determined by the decrease in chromatographic peak area and log transformed to calculate the half-life.

In Vivo Protease Resistance Assays: A key benefit of peptide stapling is the translation of in vitro protease resistance into markedly improved pharmacokinetics in vivo. Liquid chromatography/mass spectrometry-based analytical assays are used to detect and quantitate stapled peptide levels in plasma. For pharmacokinetic analysis, peptides are dissolved in sterile aqueous 5% dextrose (1 mg/mL) and administered to C57BL/6 mice (Jackson Laboratory) by bolus tail vein or intraperitoneal injection (e.g. 5, 10, 25, 50 mg/kg). Blood is collected by retro-orbital puncture at 5, 30, 60, 120, and 240 minutes after dosing 5 animals at each time point. Plasma is harvested after centrifugation (2,500×g, 5 minutes, 4° C.) and stored at −70° C. until assayed. Peptide concentrations in plasma are determined by reversed-phase high performance liquid chromatography with electrospray ionization mass spectrometric detection (Aristoteli et al., Journal of Proteome Res., 2007; Walden et al., Analytical and Bioanalytical Chem., 2004). Study samples are assayed together with a series of 7 calibration standards of peptide in plasma at concentrations ranging from 1.0 to 50.0 μg/mL, drug-free plasma assayed with and without addition of an internal standard, and 3 quality control samples (e.g. 3.75, 15.0, and 45.0 μg/mL). Standard curves are constructed by plotting the analyte/internal standard chromatographic peak area ratio against the known drug concentration in each calibration standard. Linear least squares regression is performed with weighting in proportion to the reciprocal of the analyte concentration normalized to the number of calibration standards. Values of the slope and y-intercept of the best-fit line are used to calculate the drug concentration in study samples. Plasma concentration-time curves are analyzed by standard noncompartmental methods using WinNonlin Professional 5.0 software (Pharsight Corp., Cary, NC), yielding pharmacokinetic parameters such as initial and terminal phase plasma half-life, peak plasma levels, total plasma clearance, and apparent volume of distribution.

Persistence of stapled peptides of the invention in the nasal mucosa after topical administration (i.e. nose drops) and in the respiratory mucosa after intransal application or nebulization is examined in the context of pre- and post-infection blockade of viral fusion and dissemination. Mice are exposed to single treatment by nose drop or nebulizer at a series of intervals preceding intranasal infection with SARS-CoV-2, and the duration of protection from mucosal infection (assessed histologically as described above or by PCR as describe below) is used to measure the relative mucosal stability and prophylactic efficacy of the stapled peptide constructs derivatized with PEG(n)-thiocholesterol or PEG(n)-cholesterol described herein.

In vitro Binding Assays: To assess the binding and affinity of peptidomimetic macrocycles and peptidomimetic precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules or peptides and then bound to proteins of high apparent molecular weights (e.g. FITC-labeled peptides bound to a large protein) emit higher levels of polarized fluorescence due to their slower rates of rotation upon protein binding as compared to fluorescent tracers attached to smaller molecules or peptides alone (e.g. FITC-labeled peptides that are free in solution).

In vitro Displacement Assays to Characterize Antagonists of Peptide-Protein Interactions: To assess the binding and affinity of compounds that antagonize the interaction between a peptide and an acceptor protein, a fluorescence polarization assay (FPA) utilizing a fluoresceinated peptide or peptidomimetic macrocycle derived from a template peptide sequence is used, for example. The FPA technique measures the molecular orientation and mobility using polarized light and fluorescent tracer. When excited with polarized light, fluorescent tracers (e.g., FITC) attached to molecules that are then bound to proteins with high apparent molecular weights (e.g. FITC-labeled peptides bound to a large protein) emit higher levels of polarized fluorescence due to their slower rates of rotation as compared to the FITC-derivatized molecules alone (e.g. FITC-labeled peptides that are free in solution). Compounds such as unlabeled stapled peptides and their conjugates that antagonize the interaction between the fluoresceinated peptide and an acceptor protein will be detected in a competitive binding FPA experiment and the differential potency of compounds in disrupting the interaction can be quantified and compared.

Five helix bundle protein production and fluorescence polarization assay: A C-terminal Hexa-His tagged recombinant 5-helix bundle (5HB) protein is designed containing 5 of the 6 helices that comprise the core of the SARS-CoV-2 S trimer of hairpins, connected by short peptide linkers in accordance with the design of the gp41 5-HB (Root et al. Science, 291(5505):884-8 (2001); Bird et al., J Clin Invest. 2014 May; 124(5):2113-24). The plasmid is transformed into Escherichia coli BL21 (DE3), cultured in Luria broth, and induced with 0.1 M isopropyl β-D-thiogalactoside overnight at 37° C. The cells are harvested by centrifugation for 20 minutes at 5,000 g, resuspended in buffer A (100 mM NaH2PO4, 20 mM Tris, 8 M urea; pH 7.4), and lysed by agitation at 4° C. overnight. The mixture is clarified by centrifugation (35,000 g for 30 minutes) before binding to a nickel-nitrilotriacetate (Ni-NTA) agarose (Qiagen) column at room temperature. The bound 5-HB is washed with buffer A (pH 6.3), eluted with buffer A (pH 4.5), renatured by diluting (1:2) with PBS (50 mM sodium phosphate, 100 mM NaCl; pH 7.5), and concentrated in a 10-kDa Amicon centricon (diluting and reconcentrating 7 times), yielding approximately 1 mg/ml protein solution. Purity of the protein is assessed by SDS-PAGE and determined to be >90%. Fluoresceinated derivatives of the peptides of the invention (25 nM) are incubated with 5-HB protein at the indicated concentrations in room temperature binding buffer (50 mM sodium phosphate, 100 mM NaCl; pH 7.5). Direct binding activity at equilibrium (e.g. 10 minutes) is measured by fluorescence polarization using a SpectraMax M5 microplate reader (BMG Labtech). For a competitive binding assay, a fixed concentration of FITC-peptide and 5-HB protein reflecting the EC90 for direct binding is then incubated with a serial dilution of acetylated SAH-SARS-CoV-2 peptides to generate competition curves for comparative analyses. Binding assays are run in triplicate, and K is are calculated by nonlinear regression analysis of the competition binding isotherms using Prism software (GraphPad).

Assay to screen for binding activity to the SARS-CoV-2 5 helix bundle: In some instances, the methods disclosed herein include direct and competitive screening assays. For example, methods can include determining whether an agent alters (e.g., reduces) binding of one or more of the peptides and conjugates thereof disclosed herein to SARS-CoV-2 (e.g., to SARS-CoV-2 5-helix bundle). In some instances, methods include (i) determining a level of binding between one or more of the peptides and conjugates thereof disclosed herein and SARS-CoV-2 (e.g., to SARS-CoV-2 5-helix bundle) (e.g., in the absence of an agent); and (ii) detecting the level of binding between one or more peptides (e.g., the one or more peptides of (i)) and SARS-CoV-2 (e.g., to SARS-CoV-2 5-helix bundle) in the presence of an agent, wherein a change (e.g., reduction) in the level of binding between the one or more peptides and SARS-CoV-2 (e.g., to SARS-CoV-2 5-helix bundle) indicates that the agent is a candidate agent that binds to SARS-CoV-2; and (iii) selecting the candidate agent. In some instances, step (i) includes contacting one or more peptides with SARS-CoV-2 (e.g., to SARS-CoV-2 5-helix bundle) and detecting the level of binding between one or more peptides with SARS-CoV-2 (e.g., to SARS-CoV-2 5-helix bundle). In some instances, step (ii) includes contacting the one or more peptides and the agent with SARS-CoV-2 (e.g., to SARS-CoV-2 5-helix bundle) and detecting the level of binding between one or more peptides with SARS-CoV-2 (e.g., to SARS-CoV-2 5-helix bundle). SARS-CoV-2 (e.g., to SARS-CoV-2 5-helix bundle) can be contacted with the one or more peptides and the agent at the same time or at different times (e.g., the one or more peptides can be contacted with SARS-CoV-2 (e.g., to SARS-CoV-2 5-helix bundle) before or after the agent). In some embodiments, candidate agents are administered to a suitable animal model (e.g., an animal model of COVID-19) to determine if the agent reduces a level of COVID-19 infection in the animal.

In some instances, one or both of the peptide and the SARS-CoV-2 helix bundle can include a label, allowing detection of the peptide and/or the SARS-CoV-2 helix bundle. In some instances, the peptide includes a label. In some instances, the SARS-CoV-2 helix bundle includes a label. In some instances, both the peptide and the SARS-CoV-2 helix bundle include a label. A label can be any label known in the art, including but not limited to a fluorescent label, a radioisotope label, or an enzymatic label. In some instances, the label is directly detectable by itself (e.g., radioisotope labels or fluorescent labels). In some instances, (e.g., in the case of an enzymatic label), the label is indirectly detectable, e.g., by catalyzing chemical alterations of a chemical substrate compound or composition, which chemical substrate compound or composition is directly detectable.

Competitive SARS-CoV-2 5-HB binding assay by ELISA: Microwells are coated overnight at 4° C. with 50 μl of PBS containing neutravidin (4 μg/ml). Wells are washed twice with PBS containing 0.05% Tween 20 (PBS-T), and blocked with 4% BSA in PBS-T for 45 min at 37° C. Next, 50 μl of 250 nM biotinylated-PEG2-SARS-CoV-2 HR2 (SEQ ID NO:6) is added in PBS-T with 1% BSA and incubated with shaking for 1 hr followed by 4× washes with 300 μl of PBS-T. Then, a 1:2 serial dilution of stapled peptides of the invention starting at 10 μM containing 50 nM of recombinant 5-HB in 50 μL of PBS-T with 1% BSA is added to the plate and shaken at room temperature for 2 hr followed by 4× washes with 300 μl of PBS-T. Finally, 50 μL of a 1:5000 dilution of goat polyclonal to 6× His tag-HRP conjugated is added. Following incubation at RT for 40 min, the wells are washed five times, and developed by adding 50 μl of tetramethylbenzidine (TMB) solution. After 20 min, wells containing TMB solution are stopped by adding 50 μl of H₂SO₄ (2 M), and the absorbance at 450 nm is read on a microplate reader (Molecular Devices). The concentration of competitor peptide corresponding to a half-maximal signal (IC50) is determined by interpolation of the resulting binding curve using Prism software (Graphpad). Each peptide competitor is tested in triplicate in at least two separate experiments.

Cellular Localization Assays: To measure the localization of peptides or crosslinked polypeptides on or in cells, intact cells are incubated with fluoresceinated crosslinked polypeptides derivatized with PEG(n)-thiocholesterol or PEG(n)-cholesterol (5 μM) for 4 hours in serum-free media or in media supplemented with human serum at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan® HCS Reader.

Antiviral Efficacy Assays: The efficiency of the peptide-cholesterol conjugates of the invention in preventing and treating live SARS-CoV-2 virus infection are evaluated in monolayer cell cultures. A viral detection platform has been developed for SARS-CoV-2 based on previous screens against Ebolaviruses (see, Anantpadma M. et al., Antimicrob Agents Chemother. 2016; 60(8):4471-81. Epub 2016/05/11. doi: 10.1128/AAC.00543-16. PubMed PMID: 27161622; PMCID: PMC4958205). Vero E6 cells plated in 384-well format are treated for 1 hour with a serial dilution of stapled peptides (e.g. 1-5 μM starting dose), performed in triplicate, followed by 4 hour challenge with SARS-CoV-2 (e.g., USA-WA1/2020 [wild-type], S African B.1.351 [beta strain]) or Indian B.1.617.2 [delta strain] to achieve control infection of 10-25% cells (the pre-determined optimal infectivity to assess the dynamic range of test compounds in the assay). Infected cells are then washed, fixed with 4% paraformaldehyde, rewashed in PBS, immune-stained with anti-SARS-CoV-2 nucleocapsid monoclonal antibody followed by anti-Ig secondary antibody (Alexa Fluor 488; Life Technologies), and cell bodies counterstained with HCS CellMask blue. Cells are imaged across the z-plane on a Nikon Ti Eclipse automated microscope, analyzed by CellProfiler software, and infection efficiency calculated by dividing infected by total cells. Control cytotoxicity assays are performed using Cell-Titer Glo (Promega) and LDH release (Roche) assays.

In an alternative approach, qPCR based viral detection is used in natively-susceptible human-derived Huh770 and Calu-371 cells that express ACE2, and also MatTek Life Sciences primary lung epithelial and alveolar cell models, infected with SARS-CoV-2 virus (e.g. USA-WA1/2020; Hongkon VM20001061). Cultured cells are treated for 1 hour with a serial dilution of stapled peptide-cholesterol conjugates of the invention followed by challenge with SARS-CoV-2 virus. Culture supernatants are sampled, virus lysed in the presence of RNAse inhibitor, and RT and qPCR performed as described. See Suzuki et al. J Vis Exp. 2018(141). Epub 2018/11/20. doi: 10.3791/58407. CDC-validated BHQ quenched dye pair primers are purchased from IDT and genome equivalents calculated from Ct values.

In yet another approach, antiviral activity of the stapled peptide-cholesterol conjugates of the invention are assessed using pseudotyped virus. The 293T-hsACE2 stable cell line (Cat #C-HA101) and the pseudotyped SARS-CoV-2 virus [RVP-701G (Wuhan-Hu-1) RVP-702G (D614G B.1, 20A) RVP-706G (UK variant B.1.1.7, 20I/501Y.V1) RVP-724G (South African, variant A3 B.1.351,20H/501Y.V2) RVP-763G (Indian variant, B.1.617.2) RVP-768G (BA. 1) RVP-770G (BA.2) RVP-801G (SARS-CoV-1 Urbani) RVP-1002G (VSV with MLV core) reporters are used (Integral Molecular). The neutralization assay is carried out according to the manufacturers' protocols. Pseudotyped VSV virus with an MLV core and GFP reporter is used as a viral specificity control for these assays. In brief, a serial two-fold dilution of peptide (starting dose 500 or 1000 nM) is incubated with 5 μL pseudotyped SARS-CoV-2-GFP for 1 hr at 37° C. in a 384 well black clear bottom plate followed by addition of 30 μL of 1,000 293T-hsACE2 cells in 10% FBS DMEM, phenol red free media and placed in a humidified incubator for 48 or 72 hrs. Hoechst 33342 (cell permeable nuclear dye) and DRAQ7 (cell impermeable nuclear dye) are added and the plate imaged on a Molecular Devices ImageXpress Micro Confocal Laser at 10× magnification. GFP (+) cells are counted and total GFP(+) cells or percent GFP(+) cells are plotted using Prism software (Graphpad). Cytotoxicity is determined by the ratio of DRAQ7 (+) Hoechst 33342 (+) to DRAQ7 (−) Hoechst 33342 (+) cells.

To evaluate the capacity of lead stapled peptide-cholesterol conjugates to prevent SARS-CoV-2 infection, K18-hACE2 (Jackson Laboratory) mice (n=10 per arm; 5 male, 5 female) are treated intranasally or by the oropharyngeal route with stapled peptide-cholesterol conjugate or vehicle and 4-24 hours later a viral dosage of 104 PFU is inoculated intranasally. Mice are euthanized 4 days later (peak of viremia) for evaluation by necropsy and viral load as quantitated by qPCR from supernatant samples of lung homogenates, prepared as described using a tissuelyzer (Qiagen). See Bao L et al. Nature. 2020. Epub 2020/05/08; doi: 10.1038/s41586-020-2312-y. To evaluate the capacity of lead stapled peptides to treat or mitigate established SARS-CoV-2 infection, K18-hACE2 mice (n=10 per arm; 5 male, 5 female) are inoculated intranasally at a viral dosage of 104 PFU on day 1, followed by daily oropharyngeal or intraperitoneal treatment with stapled peptide or vehicle for 10 days (days 2-12). In an alternate design, dosing is delayed until 3-5 days post-inoculation to simulate symptom- or positive test-driven initiation of therapy. Mice are continuously monitored to record body weights and clinical signs, with disease progression scored as >10% body weight loss, labored breathing, and/or failure to thrive. Doses for the most effective compound and route are then refined in both prevention and treatment studies to determine the minimum dose to protect mice. The same experimental design is used except that the 4 treatment groups (n=10; 5 male, 5 female) receive the original dose and then 3 progressively lowered doses in 4-fold increments. Alternative animal models (e.g., hamsters, ferrets) of SARS-CoV-2 infection are also employed.

Clinical Trials: To determine the suitability of the stapled peptides derivatized with PEG(n)-thiocholesterol or PEG(n)-cholesterol of the invention for treatment of humans, clinical trials can be performed. For example, patients exposed to SARS-CoV-2 infection or diagnosed with SARS-CoV-2 infection are selected and separated in treatment and one or more control groups, wherein the treatment group is administered a peptide of the invention, while the control groups receive a placebo or a known antiviral drug. The treatment safety and efficacy of the peptide-cholesterol conjugates of the invention can thus be evaluated by performing comparisons of the patient groups with respect to factors such as prevention of symptoms, time to resolution of symptoms, and/or overall infection severity. In another example, uninfected patients are identified and are given either a cross-linked polypeptide or a placebo. After receiving treatment, patients are followed. In both examples, the SARS-CoV-2-exposed patient group treated with a stapled peptide-cholesterol conjugate of the invention would avoid the development of infection, or a patient group with SARS-CoV-2 infection would show resolution of or relief from symptoms or their severity compared to a patient control group treated with a placebo.

Enumerated Embodiments: The disclosure provides the following exemplary enumerated embodiments.

Embodiment 1. A structurally-stabilized polypeptide comprising an amino acid sequence that is at least 55%, 60%, 65%, 70%, 75%, 80%, 85%, 90%, or 94% identical to sequence set forth in SEQ ID NO: 6 (DISGINASVVNIQKEIDRLNEVAKNLNESLIDLQELGK), wherein amino acids at positions of SEQ ID NO:6 selected from (wherein position 1 is the N-terminal Aspartic Acid and position 38 is the C-terminal Lysine of SEQ ID NO: 6): (i) positions 14 and 21,
(ii) positions 17 and 24,
(iii) positions 20 and 27;
(iv) positions 21 and 28; or
(v) positions 24 and 31 are replaced by α, α-disubstituted non-natural amino acids with olefinic side chains;
wherein the structurally-stabilized peptide is 38 to 60 amino acids in length, optionally 38 to 50 amino acids in length; and
wherein the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

Embodiment 2. The structurally-stabilized polypeptide of embodiment 1, wherein the amino acid sequence is at least 70% identical to the sequence set forth in SEQ ID NO: 6.

Embodiment 3. The structurally-stabilized polypeptide of embodiment 1, wherein the amino acid sequence is at least 80% identical to the sequence set forth in SEQ ID NO: 6.

Embodiment 4. The structurally-stabilized polypeptide of any one of embodiments 1 to 3, wherein the amino acid sequence comprises or consists of the amino acid sequence set forth in SEQ ID NO: 10 (DISGINASVVNIQ8EIDRLNXVAKNLNESLIDLQELGK).

Embodiment 5. The structurally-stabilized polypeptide of any one of embodiments 1 to 3, wherein the amino acid sequence comprises or consists of the amino acid sequence set forth in SEQ ID NO: 13 (DISGINASVVNIQKEI8RLNEVAXNLNESLIDLQELGK).

Embodiment 6. The structurally-stabilized polypeptide of any one of embodiments 1 to 3, wherein the amino acid sequence comprises or consists of the amino acid sequence set forth in SEQ ID NO: 17 (DISGINASVVNIQKEIDRLN8VAKNLNXSLIDLQELGK).

Embodiment 7. The structurally-stabilized polypeptide of any one of embodiments 1 to 6, wherein the structurally-stabilized polypeptide is 38 to 45 amino acids in length.

Embodiment 8. The structurally-stabilized polypeptide of any one of embodiments 1 to 7, wherein the structurally-stabilized polypeptide is 38 to 40 amino acids in length.

Embodiment 9. The structurally-stabilized polypeptide of any one of embodiments 1 to 8, wherein positions 37 and/or 38 of SEQ ID NO: 6 are not substituted.

Embodiment 10. A structurally stabilized peptide comprising at least 19 contiguous amino acids of an amino acid sequence set forth in SEQ ID NO: 10 with at least one to eighteen of the amino acids at positions 3, 4, 6, 9, 11, 13, 15, 18, 20, 22, 25, 27, 29, 32, 34, 35, 37, or 38 in SEQ ID NO: 10 substituted by any other natural or non-natural amino acid wherein the non-natural amino acids at positions 14 and 21 of SEQ ID NO: 6 are not substituted, and, wherein the structurally stabilized peptide is 19 to 45 amino acids in length and wherein the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in pseudovirus and/or live SARS-CoV-2 virus assays, optionally, wherein the 19 contiguous amino acids correspond to positions 1179 to 1197 of HR2 except that positions 1181 and 1188 are substituted by non-natural amino acids with olefinic side chains.

Embodiment 11. The structurally-stabilized peptide of embodiment 10, wherein the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

Embodiment 12. A conjugate comprising the structurally-stabilized polypeptide of any one of embodiments 1 to 11 and polyethylene glycol (PEG) and/or cholesterol, wherein the PEG and/or cholesterol are linked to the C-terminus of the structurally-stabilized polypeptide.

Embodiment 13. The conjugate of embodiment 12, comprising PEG and cholesterol.

Embodiment 14. The conjugate of embodiment 12 or 13, wherein the cholesterol is thiocholesterol.

Embodiment 15. The conjugate of embodiment 12 or 13, wherein the conjugate comprises PEG(n)-cholesterol, wherein n is 1-36, optionally wherein n is 4, 5, 6, 7, or 8.

Embodiment 16. The conjugate of any one of embodiments 12 to 14, wherein the conjugate comprises PEG(n)-thiocholesterol, wherein n is 2-36, optionally wherein n is 4, 5, 6, 7, or 8. In some cases, n=4, 5, 6, 7, 8, 10, 12, 14, 16, 18, 20, 22, 24, 26, 28, 30, 32, 34, or 36.

Embodiment 17. A conjugate comprising:
the structurally-stabilized polypeptide of SEQ ID NO: 10 (DISGINASVVNIQ8EIDRLNXVAKNLNESLIDLQELGK), and
a PEG(4)-cholesterol moiety linked to the C-terminal lysine of the structurally-stabilized polypeptide.

Embodiment 18. The conjugate of embodiment 17, wherein the PEG(4)-cholesterol moiety comprises the formula:

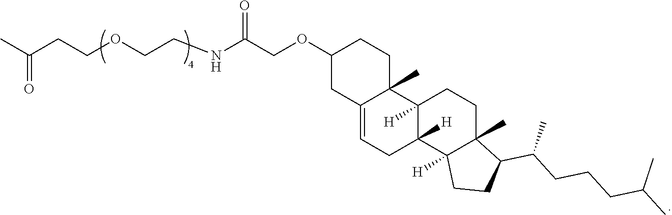

Embodiment 19. A conjugate comprising:
the structurally-stabilized polypeptide of SEQ ID NO: 10 (DISGINASVVNIQ8EIDRLNXVAKNLNESLIDLQELGK), and
a PEG(4)-thiocholesterol moiety linked to the C-terminal lysine of the structurally-stabilized polypeptide.

Embodiment 20. The conjugate of embodiment 19, wherein the PEG(4)-thiocholesterol moiety comprises the formula:

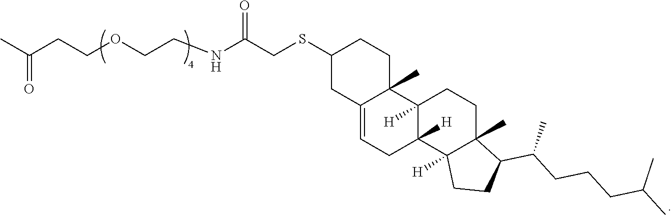

Embodiment 21. A conjugate comprising:
the structurally-stabilized polypeptide of SEQ ID NO: 10 (DISGINASVVNIQ8EIDRLNXVAKNLNESLIDLQELGK), and
a PEG(8)-cholesterol moiety linked to the C-terminal lysine of the structurally-stabilized polypeptide.

Embodiment 22. The conjugate of embodiment 21, wherein the PEG(8)-cholesterol moiety comprises the formula:

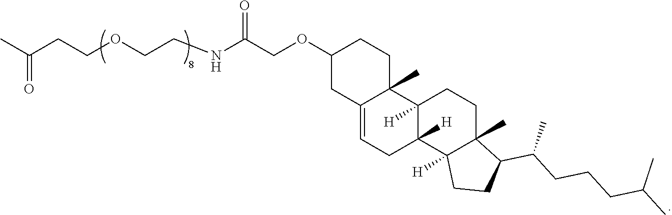

Embodiment 23. A conjugate comprising:
the structurally-stabilized polypeptide of SEQ ID NO: 10 (DISGINASVVNIQ8EIDRLNXVAKNLNESLIDLQELGK), and
a PEG(8)-thiocholesterol moiety linked to the C-terminal lysine of the structurally-stabilized polypeptide.

Embodiment 24. The conjugate of embodiment 23, wherein the PEG(8)-thiocholesterol moiety comprises the formula:

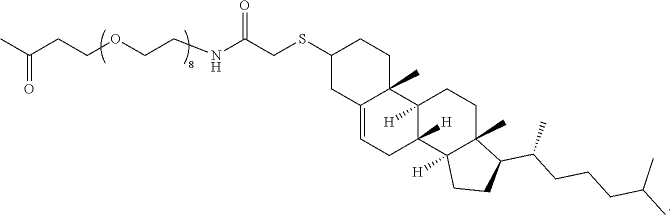

Embodiment 25. A conjugate comprising:
the structurally-stabilized polypeptide of SEQ ID NO: 10 (DISGINASVVNIQ8EIDRLNXVAKNLNESLIDLQELGK), and
a PEG(n)-cholesterol moiety linked to the C-terminal lysine of the structurally-stabilized polypeptide, wherein (n)=4, 5, 6, 7, or 8. In some cases, n=4, 5, 6, 7, 8, 10, 12, 14, 16, 18, 20, 22, 24, 26, 28, 30, 32, 34, or 36.

Embodiment 26. The conjugate of embodiment 25, wherein the PEG(n)-cholesterol moiety comprises the formula:

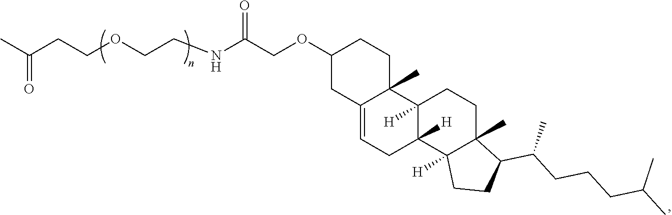

wherein n=4, 5, 6, 7, or 8. In some cases, n=4, 5, 6, 7, 8, 10, 12, 14, 16, 18, 20, 22, 24, 26, 28, 30, 32, 34, or 36.

Embodiment 27. A conjugate comprising:
the structurally-stabilized polypeptide of SEQ ID NO: 10 (DISGINASVVNIQ8EIDRLNXVAKNLNESLIDLQELGK), and
a PEG(n)-thiocholesterol moiety linked to the C-terminal lysine of the structurally-stabilized polypeptide, wherein (n)=4, 5, 6, 7, or 8. In some cases, n=4, 5, 6, 7, 8, 10, 12, 14, 16, 18, 20, 22, 24, 26, 28, 30, 32, 34, or 36.

Embodiment 28. The conjugate of embodiment 27, wherein the PEG(n)-thiocholesterol moiety comprises the formula:

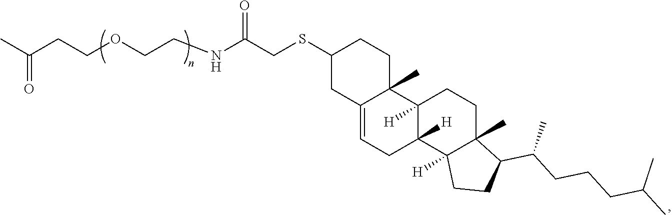

wherein n=4, 5, 6, 7, or 8. In some cases, n=4, 5, 6, 7, 8, 10, 12, 14, 16, 18, 20, 22, 24, 26, 28, 30, 32, 34, or 36.

Embodiment 29. A structurally-stabilized peptide comprising the formula:

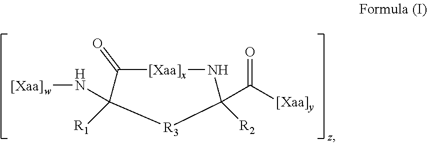

or a pharmaceutically acceptable salt thereof, wherein:
each R₁ and R₂ is H or a C1 to C10 alkyl, alkenyl, alkynyl, arylalkyl, cycloalkylalkyl, heteroarylalkyl, or heterocyclylalkyl, any of which is substituted or unsubstituted;
each R₃ is independently alkane alkylene, alkenylene, or alkynylene, any of which is substituted or unsubstituted;
z is 1, 2, 3, 4, 5, 6, 7, 8, 9, or 10;
each [Xaa]W is DISGINASVVNIQ (SEQ ID NO: 34), each [Xaa]ₓ is EIDRLN (SEQ ID NO: 35), and each [Xaa]_{y} is VAKNLNESLIDLQELGK (SEQ ID NO: 36); and
wherein the structurally-stabilized peptide inhibits infection of a cell by SARS-CoV-2 in in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

Embodiment 30. The structurally-stabilized peptide or pharmaceutically acceptable salt thereof of embodiment 29, wherein R₁ is an alkyl.

Embodiment 31. The structurally-stabilized peptide or pharmaceutically acceptable salt thereof of embodiment 29, wherein R₁ is a methyl group.

Embodiment 32. The structurally-stabilized peptide or pharmaceutically acceptable salt thereof of embodiment 29, wherein R₃ is an alkyl.

Embodiment 33. The structurally-stabilized peptide or pharmaceutically acceptable salt thereof of embodiment 29, wherein R₃ is a methyl group.

Embodiment 34. The structurally-stabilized peptide or pharmaceutically acceptable salt thereof of embodiment 29, wherein R₂ is an alkenyl.

Embodiment 35. The structurally-stabilized peptide or pharmaceutically acceptable salt thereof of anyone of embodiments 29 to 34, wherein the pharmaceutically acceptable salt comprises hydrochloride, sodium, sulfate, acetate, phosphate or diphosphate, chloride, potassium, maleate, calcium, citrate, mesylate, nitrate, tartrate, aluminum, gluconate, and any combination thereof.

Embodiment 36. The structurally-stabilized peptide or pharmaceutically acceptable salt thereof of any one of embodiments 29 to 35, which is at most 50 amino acids in length, optionally at most 45 amino acids in length.

Embodiment 37. The structurally-stabilized peptide or pharmaceutically acceptable salt thereof of any one of embodiments 29 to 36, which is 38 amino acids in length.

Embodiment 38. A conjugate comprising the structurally-stabilized peptide or pharmaceutically acceptable salt thereof of any one of embodiments 29 to 37 and PEG and/or cholesterol.

Embodiment 39. The conjugate of embodiment 38, comprising PEG and cholesterol.

Embodiment 40. The conjugate of embodiment 38 or 39, wherein the cholesterol is thiocholesterol.

Embodiment 41. The conjugate of embodiment 38 or 39, wherein the conjugate comprises PEG(n)-cholesterol, optionally wherein n is 1-36, optionally wherein n is 4, 5, 6, 7, or 8.

Embodiment 42. The conjugate of any one of embodiments 38 to 40, wherein the conjugate comprises PEG(n)-thiocholesterol, wherein n is 1-36, optionally wherein n is 4, 5, 6, 7, or 8.

Embodiment 43. A pharmaceutical compound comprising the structurally-stabilized peptide, pharmaceutically acceptable salt thereof, or the conjugate, of any one of embodiments 1 to 42 and a pharmaceutically acceptable carrier.

Embodiment 44. A method of treating a coronavirus infection in a human subject in need thereof, the method comprising administering to the human subject a therapeutically-effective amount of the structurally-stabilized peptide, the pharmaceutically acceptable salt thereof, or the conjugate of any one of embodiments 1 to 43.

Embodiment 45. A method of preventing a coronavirus infection in a human subject in need thereof, the method comprising administering to the human subject a therapeutically-effective amount of the structurally-stabilized peptide, the pharmaceutically acceptable salt thereof, or the conjugate of any one of embodiments 1 to 43.

Embodiment 46. A method of treating a coronavirus infection in a subject in need thereof, the method comprising administering to the subject a therapeutically-effective amount of the structurally-stabilized peptide, the pharmaceutically acceptable salt thereof, or the conjugate of any one of embodiments 1 to 43, wherein the subject is selected from a cow, a pig, a horse, a cat, a dog, a rat, a mouse, or a bat.

Embodiment 47. A method of preventing a coronavirus infection in a subject in need thereof, the method comprising administering to the subject a therapeutically-effective amount of the structurally-stabilized peptide, the pharmaceutically acceptable salt thereof, or the conjugate of any one of embodiments 1 to 43, wherein the subject is selected from a cow, a pig, a horse, a cat, a dog, a rat, a mouse, or a bat.

Embodiment 48. The method of embodiment 46 or 47, wherein the coronavirus infection is by a betacoronavirus.

Embodiment 49. The method of any one of embodiments 46 to 48, wherein the coronavirus infection is caused by an infection by SARS-CoV-2.

Embodiment 50. The method of any one of embodiments 46 to 49, wherein the coronavirus infection is caused by an infection by a variant of SARS-CoV-2.

Embodiment 51. The method of embodiment 50, wherein the variant is selected from Wuhan-Hu-1, B.1.427/B.1.429, B.1.617.2, D614G B.1, or Brazilian variant P.1.

Embodiment 52. A method of making a structurally-stabilized peptide, the method comprising: (a) providing a peptide having the sequence set forth in SEQ ID NO: 6 or a variant thereof, and (b) cross-linking the peptide, and optionally purifying the structurally-stabilized peptide.

Embodiment 53. The method of embodiment 52, wherein cross-linking the peptide is by a ruthenium catalyzed metathesis reaction.

Embodiment 54. The method of embodiment 52 or 53, further comprising formulating the structurally-stabilized peptide as a sterile pharmaceutical composition.

Embodiment 55. A method of synthesizing a conjugate comprising the structurally-stabilized polypeptide of any one of embodiments 1 to 11 or 29 to 37, the method comprising (a) providing the structurally-stabilized polypeptide; and (b) derivatizing a resin bound amine of the structurally-stabilized polypeptide with PEG and/or cholesterol containing a carboxylic acid on a resin.

Embodiment 56. A method of synthesizing a conjugate comprising a heptad repeat domain 2 (HR2) polypeptide, the method comprising (a) providing the HR2 polypeptide; and (b) derivatizing a resin bound amine of the HR2 polypeptide with PEG and/or cholesterol containing a carboxylic acid on a resin.

Embodiment 57. A method of synthesizing a conjugate comprising the structurally-stabilized polypeptide of any one of embodiments 1 to 11 or 29 to 37, the method comprising (a) providing the structurally-stabilized polypeptide; and (b) derivatizing a resin bound amine of the structurally-stabilized polypeptide with PEG and/or thiocholesterol containing a carboxylic acid on a resin.

Embodiment 58. A method of synthesizing a conjugate comprising an HR2 polypeptide, the method comprising (a) providing the HR2 polypeptide; and (b) derivatizing a resin bound amine of the HR2 polypeptide with PEG and/or thiocholesterol containing a carboxylic acid on a resin.

Embodiment 59. The method of any one of embodiments 55 to 58, wherein the derivatizing step comprises incorporating the carboxy thiocholesterol or carboxy cholesterol by solid phase synthesis by the steps of:
dissolving thiocholesterol in dichloromethane (DCM) or cholesterol in tetrahydrofuran (THF), thereby generating a solution; and
adding, in order, a base, t-butyl ester of bromoacetic acid, and trifluoroacetic acid to the solution; or alternatively the combined steps can be done sequentially after workup or column chromatography.

Embodiment 60. The method of any one of embodiments 55 to 59, wherein the derivatizing step further comprises:
treating the structurally-stabilized polypeptide bound to the resin with piperidine in a solution comprising dimethylformamide (DMF);
capping the N-terminus of the structurally-stabilized polypeptide with acetic anhydride;
deprotecting the C-terminus of the structurally-stabilized polypeptide with hydrazine in DMF;
acylating the structurally-stabilized polypeptide with an Fmoc-protected PEG(n) amino acid;
crosslinking the structurally-stabilized polypeptide; and
isolating the structurally-stabilized polypeptide from the resin.

Embodiment 61. The method of embodiment 60, wherein n=1-36.

Embodiment 62. The method of embodiment 60 or 61, wherein n=4, 5, 6, 7, or 8.

Embodiment 63. The methods of any one of embodiments 55 to 62, wherein the C-terminal lysine of SEQ ID NO: 6 is substituted with a resin-bound amine, optionally wherein the C-terminal lysine of SEQ ID NO: 6 is further substituted with a resin-bound carboxylic acid or thiol.

Embodiment 64. The method of any one of embodiments 55 to 63, wherein the cholesterol is thiocholesterol.

Embodiment 65. The method of any one of embodiments 55 to 63, wherein the conjugate comprises PEG(n)-cholesterol, wherein n is 1-36, optionally wherein n is 4, 5, 6, 7, or 8.

Embodiment 66. The method of any one of embodiments 55 to 64, wherein the conjugate comprises PEG(n)-thiocholesterol, wherein n is 1-36, optionally wherein n is 4, 5, 6, 7, or 8.

Embodiment 67. The method of any one of embodiments 55 to 66, wherein cross-linking the peptide is by a ruthenium catalyzed metathesis reaction.

Embodiment 68. A nanoparticle composition comprising the structurally-stabilized peptide, the pharmaceutically acceptable salt thereof, or the conjugate of any one of embodiments 1 to 43, optionally wherein the nanoparticle composition is a PLGA nanoparticle, and further optionally, wherein the nanoparticle composition comprises a lactic acid:glycolic acid ratio of the PLGA nanoparticle in the range of 2:98 to 100:0.

Embodiment 69. The nanoparticle composition of embodiment 68, further comprising chitosan, a dextrin, or both.

Embodiment 70. A linker comprising a PEG(n)-thiocholesterol comprising the formula:

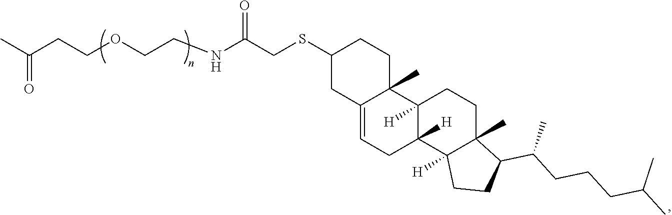

wherein n is 1-36.

Embodiment 71. A linker comprising a PEG(n)-cholesterol comprising the formula:

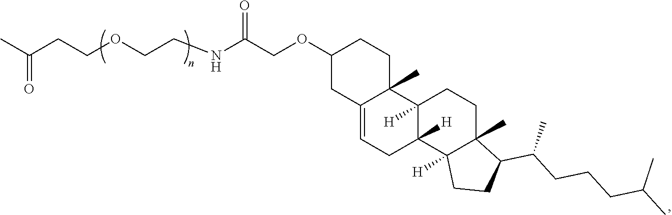

wherein n is 1-36.

Embodiment 72. The linker of embodiment 70 or 71, wherein n is 4, 5, 6, 7, or 8.

Embodiment 73. The linker of any one of embodiments 70 to 72, further comprising a lysine affixed to the PEG.

Embodiment 74. The structurally-stabilized peptide of any one of embodiments 1 to 11 or the conjugate of any one of embodiments 12 to 28, wherein:
8=(R)-α-(7′-octenyl)alanine or (R)-α-(4′-pentenyl)alanine; and
X=(S)-α-(4′-pentenyl)alanine or (S)-α-(7′-octenyl)alanine, respectively.

Embodiment 75. The structurally-stabilized peptide of any one of embodiments 1 to 11 or the conjugate of any one of embodiments 12 to 28, wherein:
8=(R)-α-(7′-octenyl)alanine; and
X=(S)-α-(4′-pentenyl)alanine.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Design and Synthesis of Stapled SARS-CoV-2 Peptides Derivatized with C-Terminal PEG(n)-Thiocholesterol or PEG(n)-Cholesterol Moieties

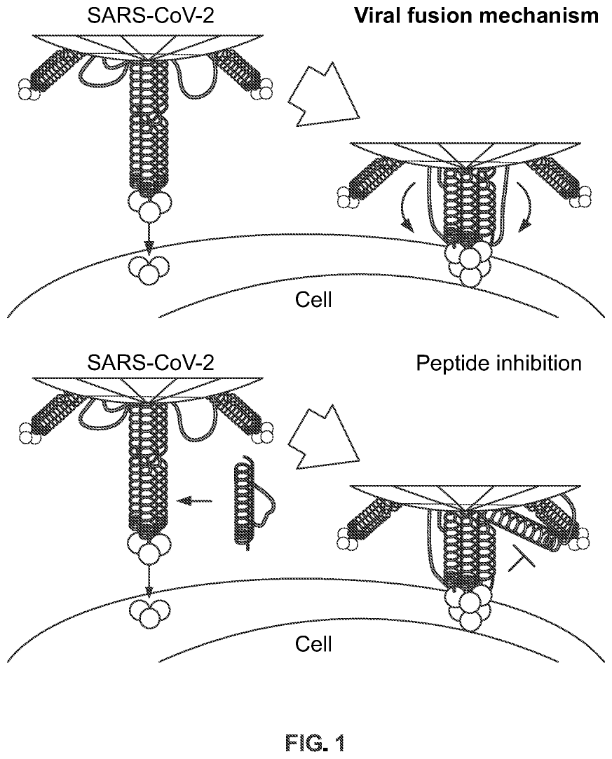

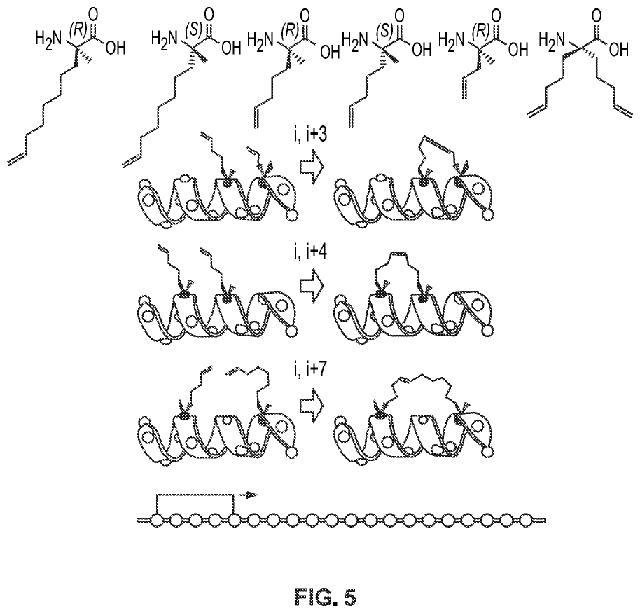

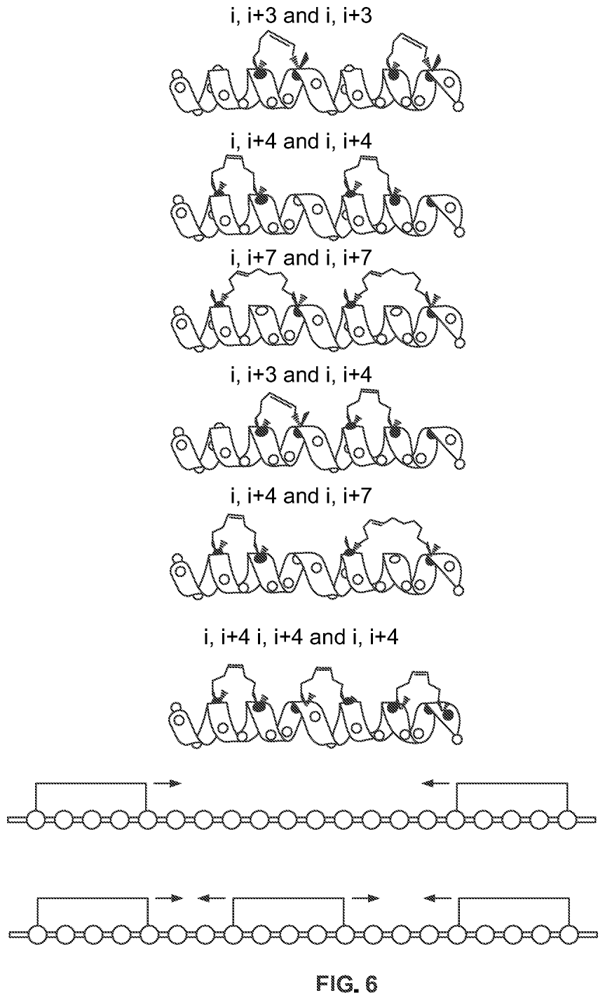

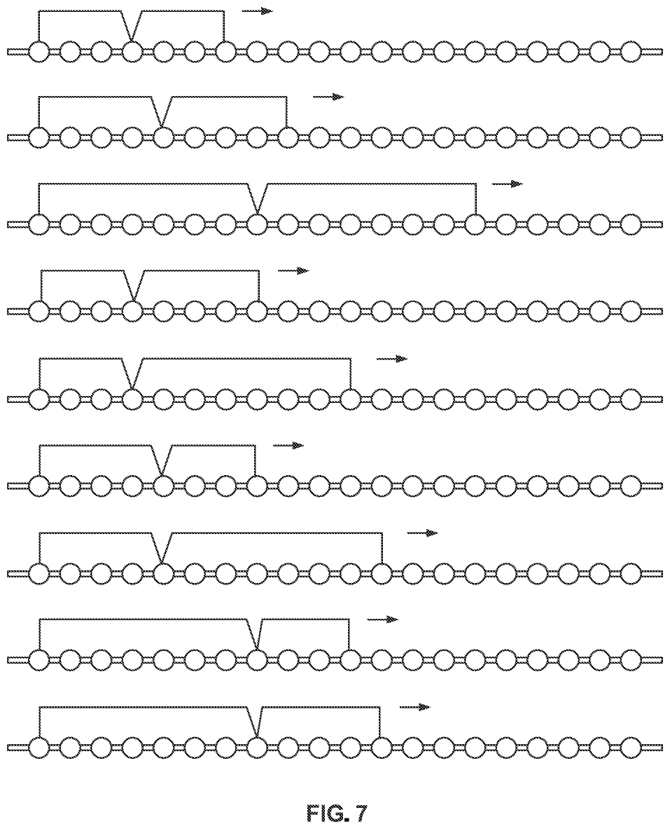

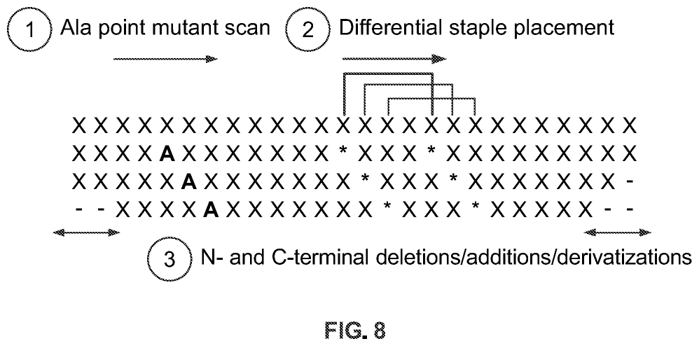

To design peptides that could block the fusion of the coronavirus to a host cell (FIG. 1), a series of stapled peptides bearing differentially localized chemical staples and derivatized with PEG(n)-thiocholesterol or PEG(n)-cholesterol moieties at the C-termini were designed and then synthesized on resin by solid phase synthesis. The differentially localized chemical staples were located within the SARS-CoV-2 HR2 domain (i.e., amino acids 1168-1205) of the sequence of the surface (S) glycoprotein of the severe acute respiratory syndrome coronavirus 2 (see, FIGS. 2-4), and preferably within the alpha-helical region (i.e., amino acids 1179-1197; see, FIG. 9), by replacing native residues with α, α-disubstituted non-natural olefinic residues (e.g., “X” for S-pentenyl alanine and “8” for R-octenyl alanine installed at select i, i+7 positions or “X” for S-pentenyl alanine installed at each of select i, i+4 positions) and combinations thereof in the form of double staples or stitches, followed by ruthenium-catalyzed olefin metathesis (see, FIGS. 5-7). Our approach to designing, synthesizing, and identifying optimal stapled peptide constructs to target the SARS-CoV-2 fusion apparatus includes the generation of Ala scan (e.g. mutants), staple scan, and variable N- and C-terminal deletion, addition, and derivatization libraries for conjugation to PEG-thiocholesterol or PEG-cholesterol moieties (see, FIG. 8). Some preferred designs incorporate staples on the non-interacting amphiphilic face of the core SARS-CoV-2 HR2 helix (e.g., SEQ ID NO: 4) or at positions at the border of the hydrophobic interaction face with the amphiphilic face of the helix (see, FIG. 9).

Stapled SARS-CoV-2 HR2 constructs bearing C-terminal derivatization with PEG(n)-thiocholesterol or PEG(n)-cholesterol moieties were designed by replacing two naturally occurring amino acids with the non-natural (R)-2-(((9H-fluoren-9-yl)methoxy)carbonylamino)-2-methyl-dec-9-enoic acid (R₈) and S-2-(4′-pentenyl) alanine (S5) amino acids at i, i+7 positions (i.e. flanking 7 amino acids) to generate a staple spanning two α-helical turns, or with two S5 non-natural amino acids at i, i+4 positions to generate a staple spanning one α-helical turn. Asymmetric syntheses of α, α-disubstituted amino acids were performed as previously described in detail (Schafmeister et al., J. Am. Chem. Soc., 2000; Walensky et al., Science, 2004; Bird et al. Current Protocols in Chemical Biology, 2011, each of which is incorporated by reference in its entirety).

“Staple scanning” was performed to respectively identify residues and binding surfaces critical for interaction, which dictates the design of optimized constructs and negative control mutants. The peptide N-termini were capped with acetyl or a fluorophore (e.g. FITC, rhodamine), depending upon the experimental application.

Doubly stapled peptides were generated by installing two-S5-S5, two R₈—S5, or other combinations of crosslinking non-natural amino acids. Multiply stapled or stitched peptides are generated using similar principles.

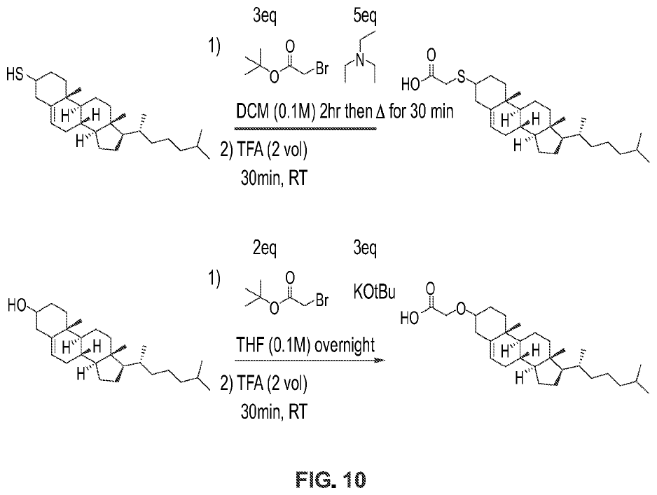

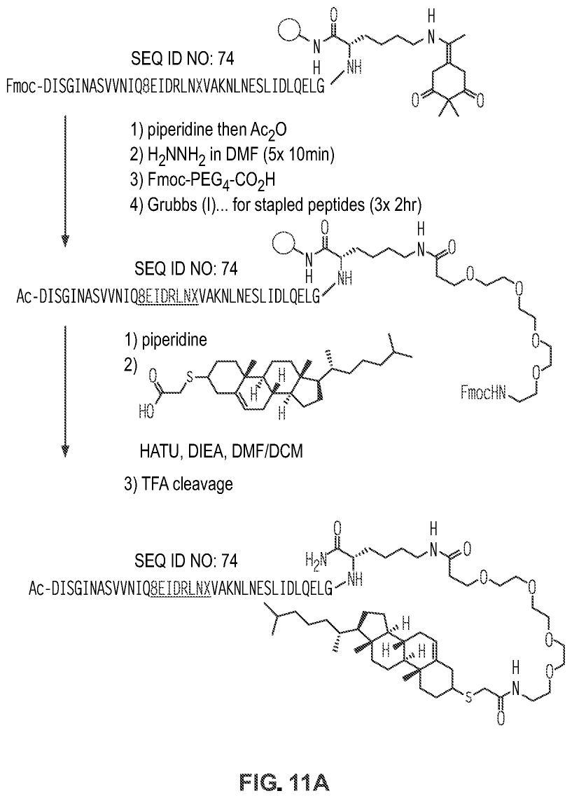

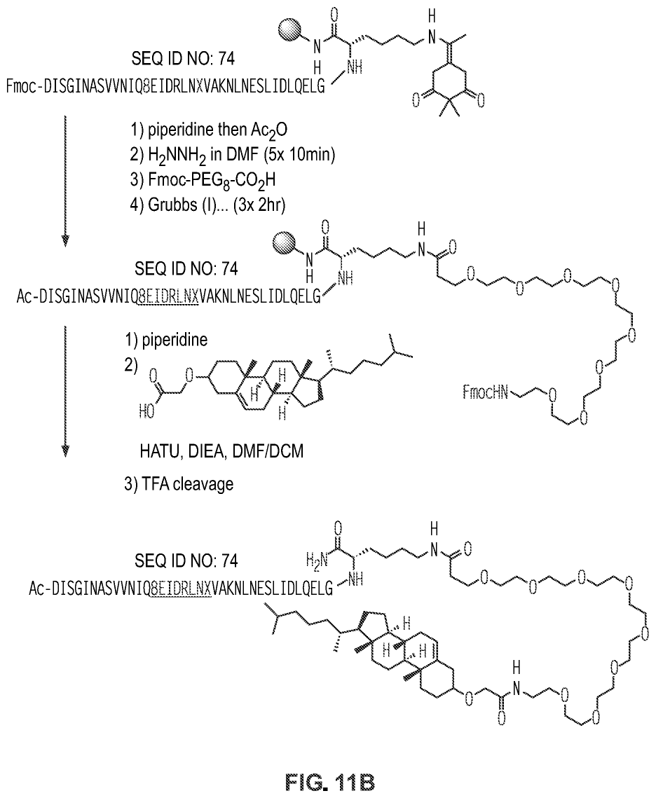

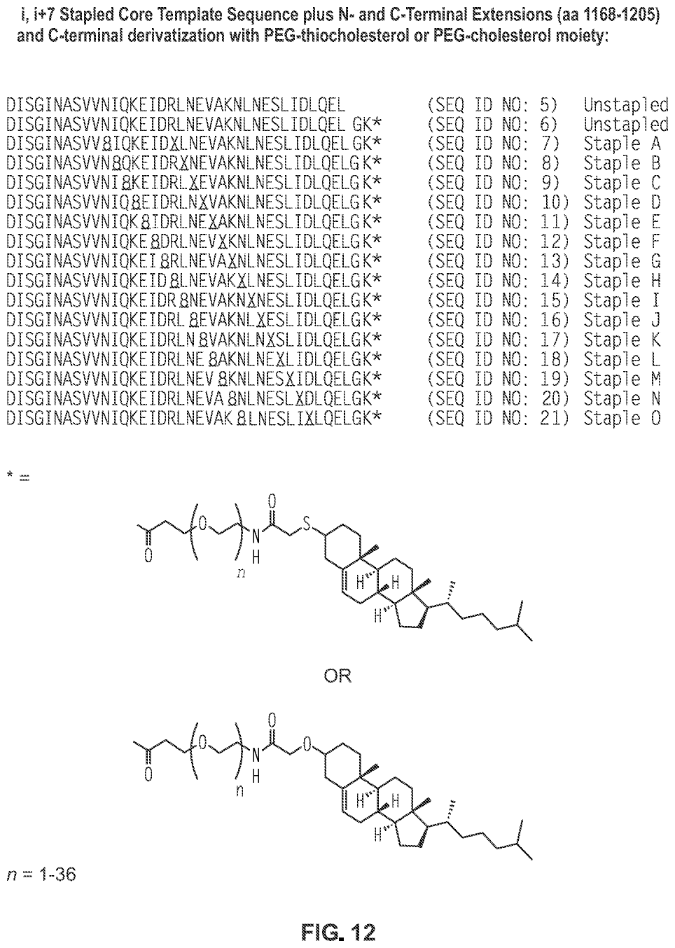

To enable peptide derivatization with thiocholesterol or cholesterol on resin, carboxy-thiocholesterol or carboxy-cholesterol were synthesized according to the procedure described above (see, Methods and FIG. 10). The completed resin-bound peptide was capped with an acetyl group (by use of acetic anhydride) followed by deprotection of the C-terminal side chain lysine amine by treatment with 2% hydrazine. The amine was acylated with an Fmoc-protected PEG(n) amino acid (e.g., n=1-36; e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) at which point the olefins were crosslinked by treating with Grubbs(I) catalyst. The Fmoc was removed from the C-terminal NH of the PEG(n) amino acid and the amine acylated with carboxy-thiocholesterol or carboxy-cholesterol. The final peptide product was obtained after peptide deprotection and cleavage, and purification by reverse phase high performance liquid chromatography/mass spectrometry (LC/MS). See the full synthetic schemas in FIG. 11A-11B. Exemplary i, i+7 stapled SARS-CoV-2 HR2 peptides derivatized with PEG(n)-thiocholesterol and PEG(n)-cholesterol generated by use of the synthetic schemas are listed in FIG. 12 (SEQ ID NOs: 7-21).

### Example 2: Identifying Optimally Stapled SARS-CoV-2 HR2 Peptides Bearing a C-Terminal PEG4-Thiocholesterol to Achieve Anti-Viral Activity in Pseudotype and Live Virus Assays

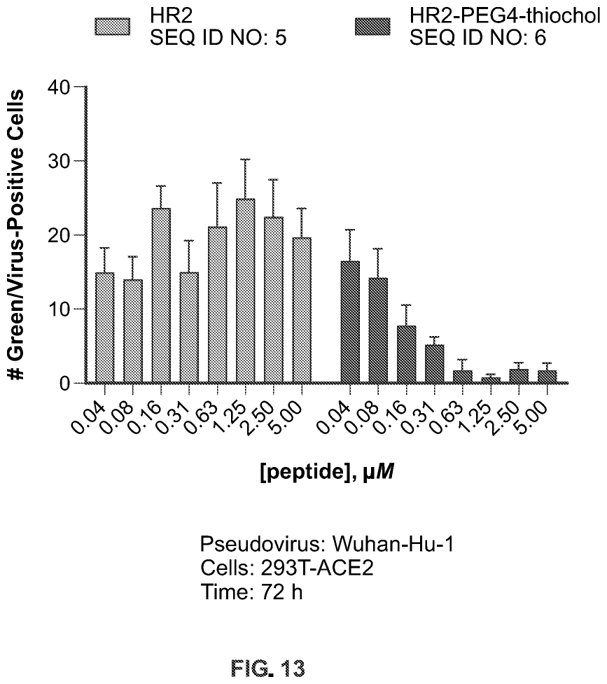

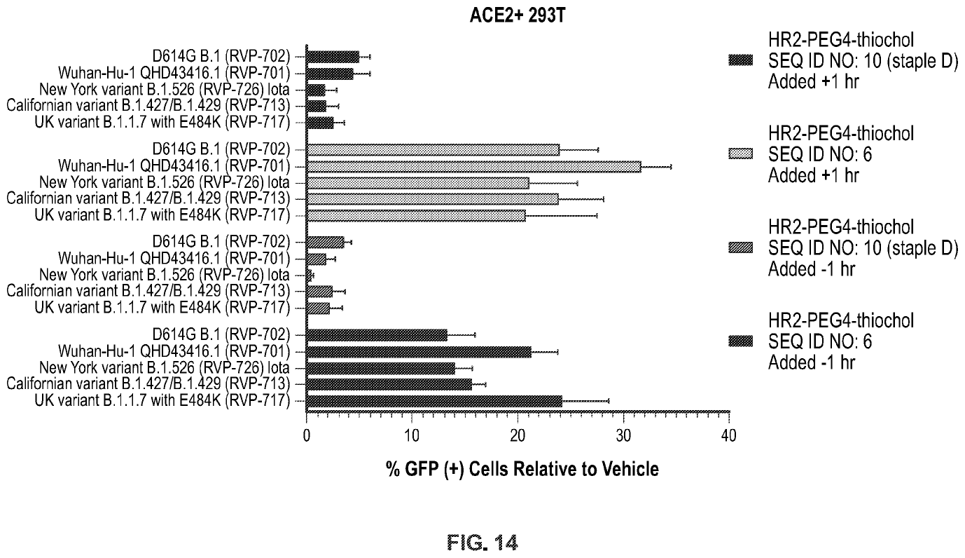

We first demonstrated that applying our chemical schema to append a PEG4-thiocholesterol moiety to the C-terminus of SEQ ID NO: 5 (unstapled peptide with C-terminal amide) to generate SEQ ID NO: 6 (unstapled peptide with C-terminal PEG4-thiocholesterol) transformed an inactive HR2 peptide into an active construct that demonstrated dose-responsive anti-viral activity in a pseudoviorus assay (see Methods) using Wuhan-Hu-1 pseudovirus, 293T-ACE2 cells, and peptide serial 2-fold dilution starting at 500 nM (see, FIG. 13). We then demonstrated that a stapled HR2 peptide bearing the PEG4-thiocholesterol moiety appended on-resin (Staple D, SEQ ID NO: 10) exhibited consistent and potent anti-viral activity in the pseudovirus assay whether the cells were treated with peptide (1 M) before or after viral inoculation across a series of SARS-CoV-2 pseudovirus variants, including D614G B.1, Wuhan-Hu-1, B.1.526, B.1.427, B.1.1.7 (see, FIG. 14). In contrast, the corresponding unstapled peptide bearing the PEG4-thiocholesterol moiety (SEQ ID NO: 6) was ineffective when applied after viral inoculation and exhibited less anti-viral activity compared to the stapled sequence even when applied before viral inoculation with the same series of SARS-CoV-2 pseudovirus variants (see, FIG. 14).

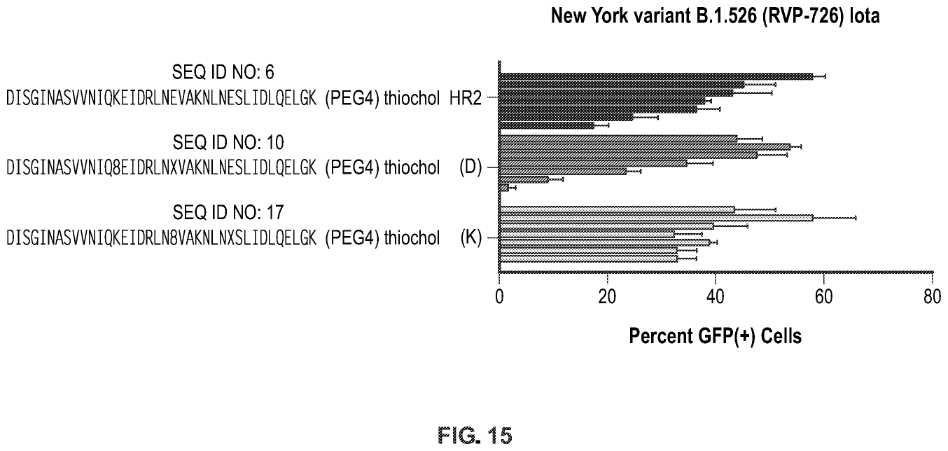

We compared the unstapled (SEQ ID NO: 6) and two stapled constructs bearing i, i+7 staples (SEQ ID NOs: 10 and 17) on the non-interacting hydrophilic face of the HR2 helix in the pseudoviral assay (pseudovirus: B.1.526; cells: 293T-ACE2; serial dilution starting at 1 mM). The stapled HR2 peptide of SEQ ID NO:10 (Staple D) showed the most potent, dose-responsive anti-viral activity followed by the unstapled peptide of SEQ ID NO: 6; notably, the stapled peptide of SEQ ID NO: 17 (Staple K) was the least active in the pseudovirus assay despite its staple also localized to the non-interacting face of the HR2 helix (see, FIG. 15). These data underscore the unpredictability of locating the optimal staple position(s), which must be determined experimentally.

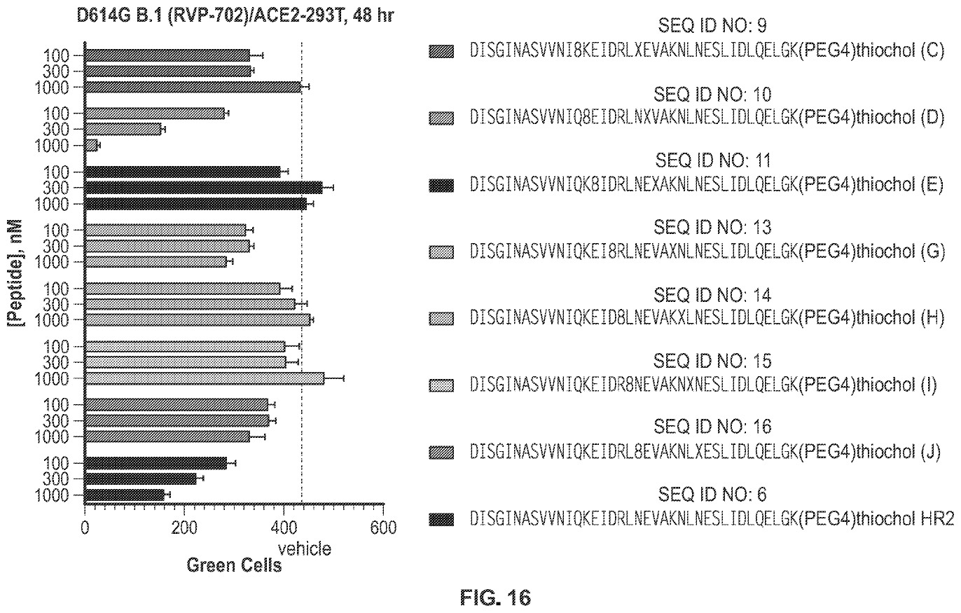

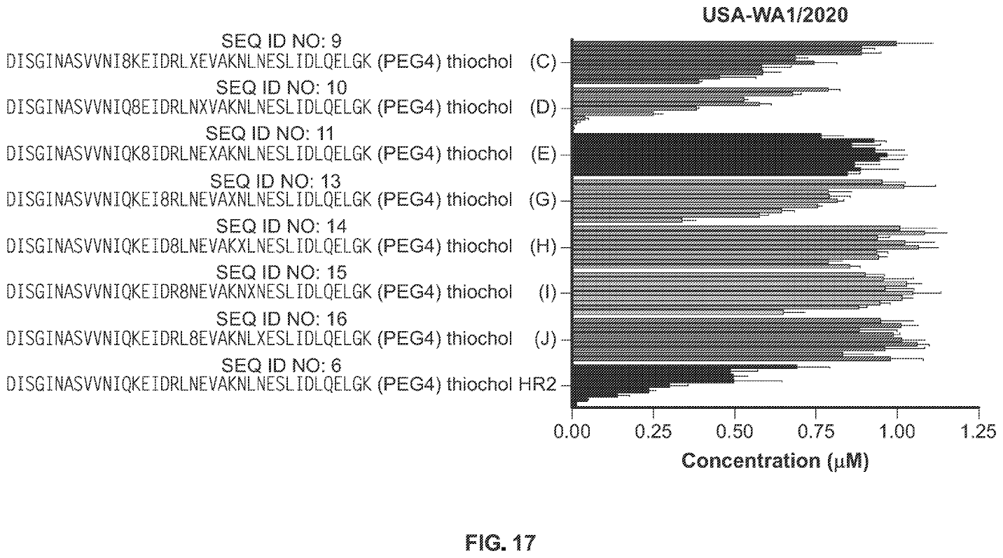

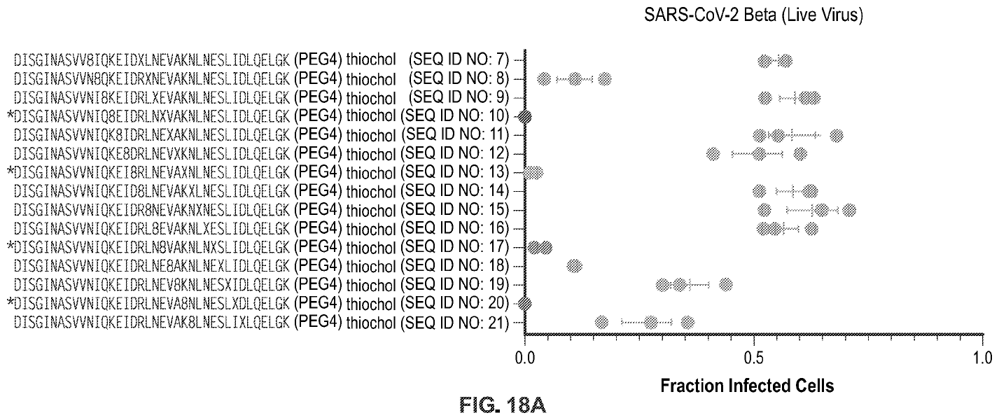

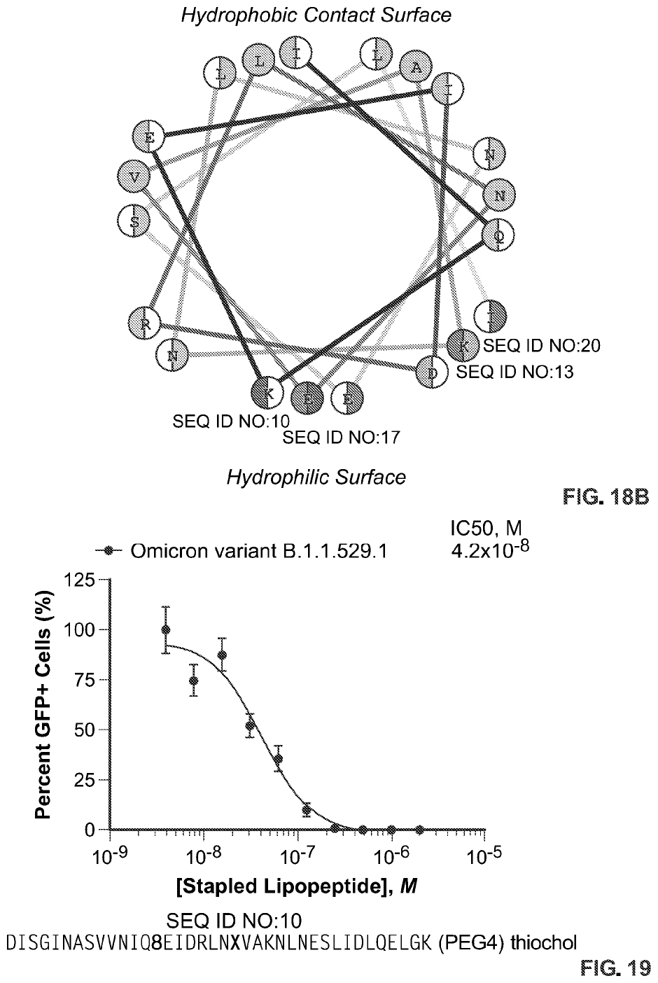

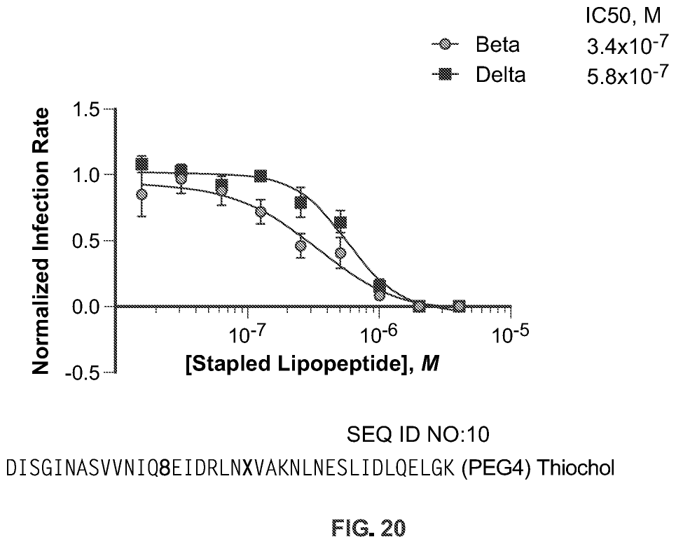

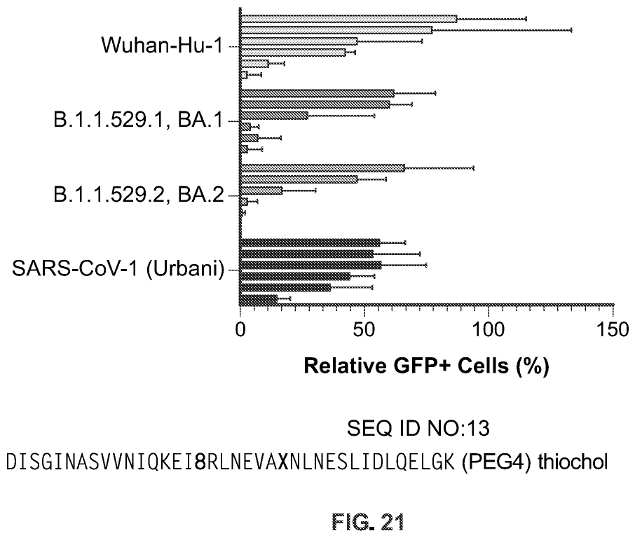

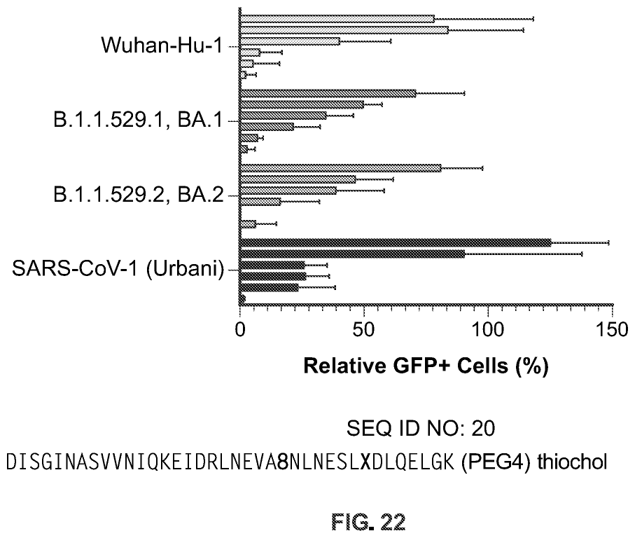

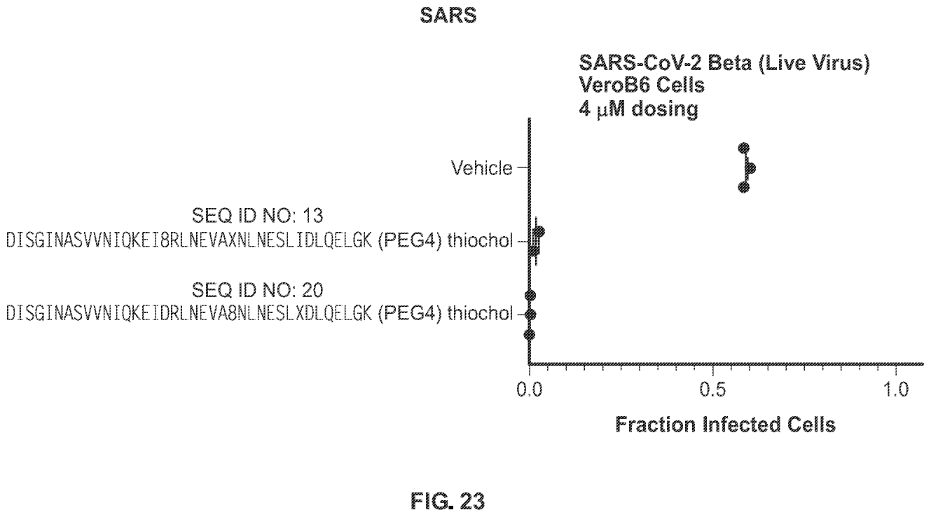

To expand the analysis, we next evaluated the differential antiviral activity of a series of i, i+7 stapled HR2 peptides bearing a C-terminal PEG4-thiocholesterol moiety, as appended on-resin. Whereas peptides of SEQ ID NOs: 11, 14, and 15 (Staples E, H, I, respectively) showed little to no activity and peptides of SEQ ID NOs: 13 and 16 (Staples G and J, respectively) exhibited moderate activity, the peptide of SEQ ID NO: 10 (Staple D) stood out as having uniquely potent activity among the various stapled HR2 peptides in the SARS-CoV-2 pseudovirus assay (pseudovirus: D614G B.1; cells: 293T-ACE2; peptide doses of 100, 300, 1000 nM) (see, FIG. 16). We then sought to corroborate this hierarchy of peptide activity in a live SARS-CoV-2 assay. We tested the differential antiviral activity of the same series of i, i+7 stapled HR2 peptides bearing a C-terminal PEG4-thiocholesterol moiety. Whereas peptides of SEQ ID NOs: 11, 14, 15 (Staples E, H, I) showed little to no activity and peptides of SEQ ID NOs: 13, 16 (Staples G, J) exhibited moderate activity, the peptide of SEQ ID NO: 10 (Staple D), consistent with the pseudovirus assays results shown in FIG. 16, stood out as having uniquely potent activity among the various stapled HR2 peptides in this SARS-CoV-2 live virus assay (live virus: USA-WA1/2020; cells: VeroB6; peptide dose-range 8-1000 nM) (see, FIG. 17). In performing a complete i, i+7 staple scan through the alpha-helical portion of the HR2 sequence and then testing the resultant constructs in a live virus assay using the SARS-CoV-2 Beta strain, we again found that SEQ ID NO:10 (Staple D), demonstrated among the most potent antiviral activity, along with three additional staple positions (SEQ ID NOs: 13, 17, and 20 with respective staple positions G, K, and N) (FIG. 18A). The select few constructs (SEQ ID NOs: 10, 13, 17, 20) that afford potent antiviral activity discretely colocalize to a focal region of the HR2 alpha-helical surface, as demonstrated by a helical wheel depiction (FIG. 18B). Among the SARS-CoV-2 variants tested in a pseudoviral assay, the stapled lipopeptide corresponding to SEQ ID NO:10 (Staple D) bearing a C-terminal PEG4 thiocholesterol moiety was most active against the Omicron variant (FIG. 19), with potent antiviral activity also demonstrated against SARS-CoV-2 live viral strains, including Beta and Delta (FIG. 20). Additional constructs, corresponding to SEQ ID NOs: 13 and 20 bearing a C-terminal PEG4 thiocholesterol moiety also demonstrated consistent antiviral activity against a diversity of SARS-CoV-2 variant and SARS-CoV-1 pseudoviruses and live SARS-CoV-2 Beta strain virus (FIGS. 21-23). The tolerance for mutagenesis within the HR2 sequence, which alternatively has contacts with the HR1 core or is solvent exposed (FIG. 24A), was demonstrated for exemplary alanine substitutions in SEQ ID NO:10 bearing a C-terminal PEG4 thiocholesterol moiety, as revealed by retention of antiviral potency in a SARS-CoV-1 pseudoviral assay (FIG. 24B).

Taken together, these data show that (1) appending a PEG4-thiocholesterol moiety using on-resin methodology to the C-terminus of an HR2 peptide can endow potent, dose-responsive antiviral activity; (2) stapling can not only enhance peptide activity compared to the unstapled analog but also achieve antiviral activity whether administered before or after viral inoculation in culture; and (3) corroborative pseudoviral and live virus assays effectively identified a select few uniquely potent stapled HR2 peptides (SEQ ID NOs: 10, 13, 17, and 20) among a complete panel of differentially i, i+7 stapled peptides with lesser or no activity, underscoring that discerning the optimal staple position to achieve potent antiviral activity is unpredictable and must be determined experimentally in SARS-CoV-2 antiviral assays.

### Example 3: Determining the Optimal PEG Linker Length within Stapled SARS-CoV-2 HR2 Peptides Bearing a C-Terminal PEG(n)-Thiocholesterol to Achieve Anti-Viral Activity in Pseudotype and Live Virus Assays

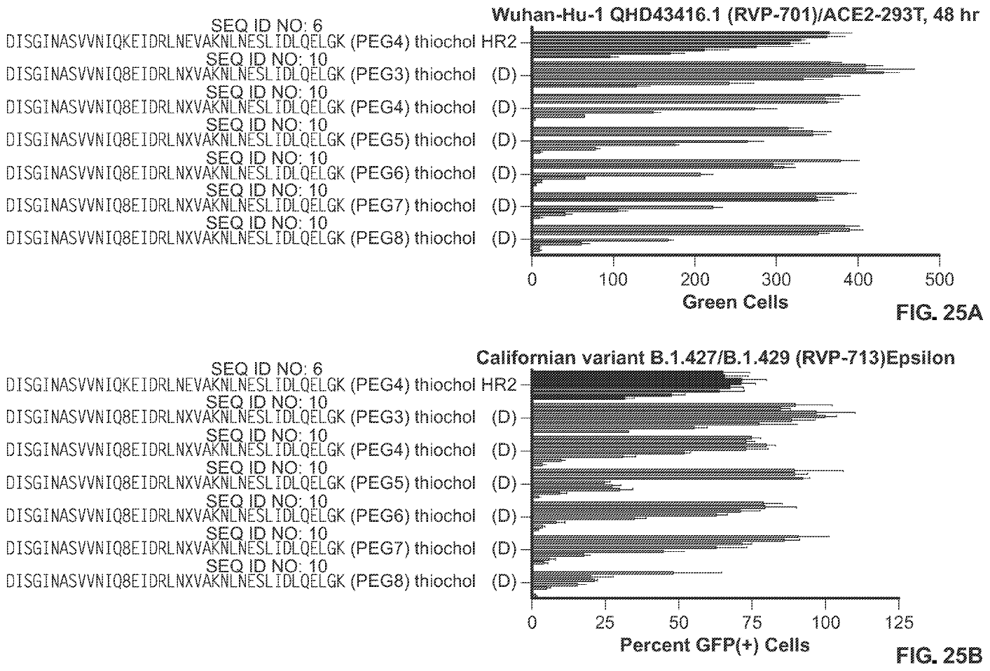

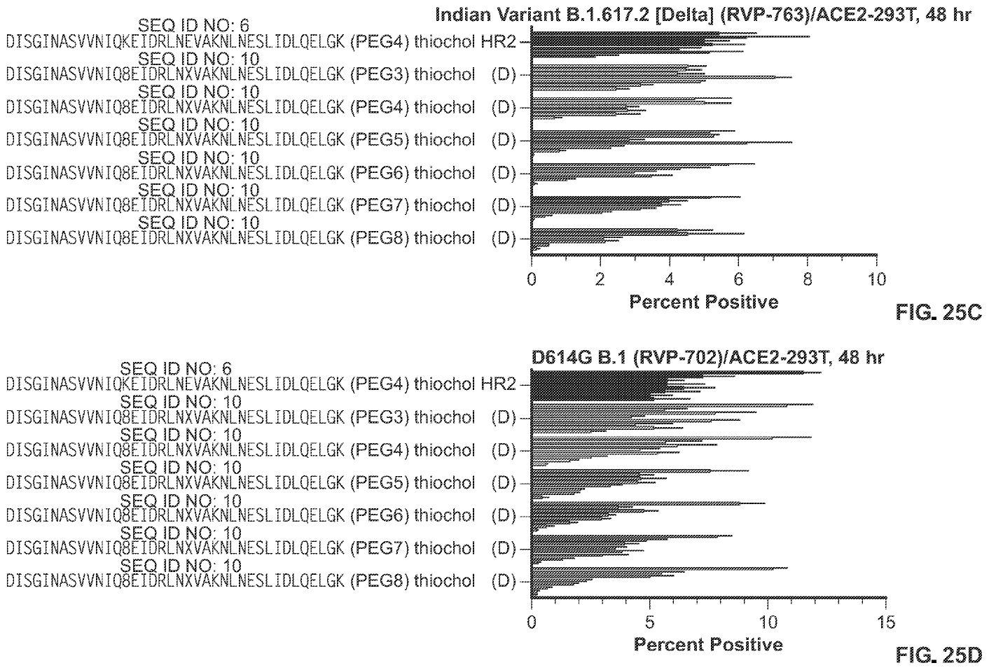

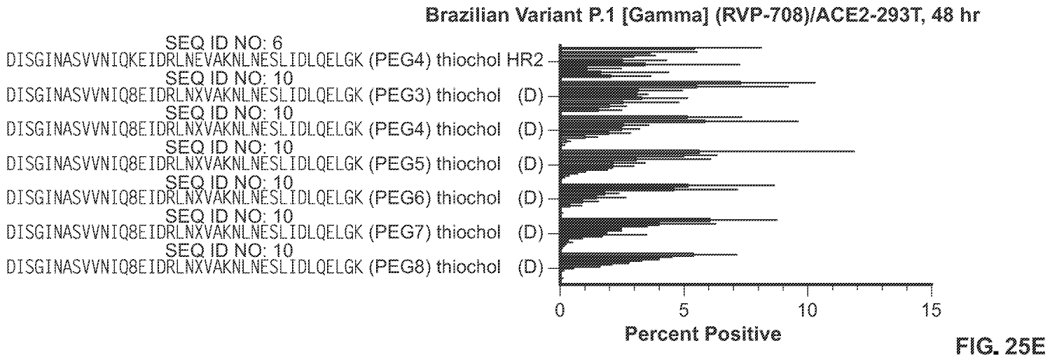

To determine the optimal PEG-chain length to link the stapled HR2 peptide to the thiocholesterol or cholesterol moiety, a series of PEG(n) analogs were generated according to the above-described synthetic method, where n equals 3, 4, 5, 6, 7, and 8. The antiviral activity of this series of i, i+7 stapled HR2 peptides bearing SEQ ID NO: 10 (Staple D) and thiocholesterol spaced by variable length PEG-linkers was examined in a pseudovirus assay across a series of five SARS-CoV-2 variants (pseudoviruses: Wuhan-Hu-1, B.1.427/B.1.429, B.1.617.2, D614G B.1, Brazilian variant P.1; cells: 293T-ACE2; serial dilution starting at 1 μM). The peptide of SEQ ID NO: 10 (Staple D) bearing a PEG8 linker exhibited the most potent, dose-responsive activity in this pseudovirus assay (see, FIGS. 25A-25E).

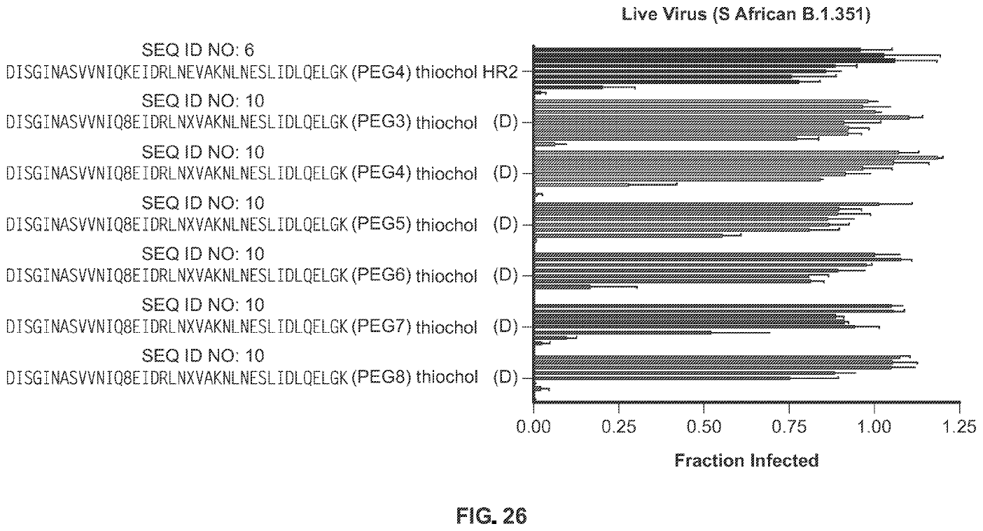

We then sought to corroborate the hierarchy of peptide activity based on PEG(n) linker length in a live SARS-CoV-2 assay. We tested the differential antiviral activity of the same series of i, i+7 stapled HR2 peptides of SEQ ID NO: 10 (Staple D) bearing a PEG(n) linker of n=3, 4, 5, 6, 7, or 8 PEG moieties and appended C-terminal thiocholesterol, as constructed on-resin, in a SARS-CoV-2 live virus assay (live virus: S. African B.1.351; cells: VeroB6; peptide serial 2-fold dilution dose-range from 1000 to 4 nM). As for the pseudovirus assay, the construct bearing a PEG8 linker moiety exhibited the most potent, dose-responsive activity in this SARS-CoV-2 live virus assay (see, FIG. 26).

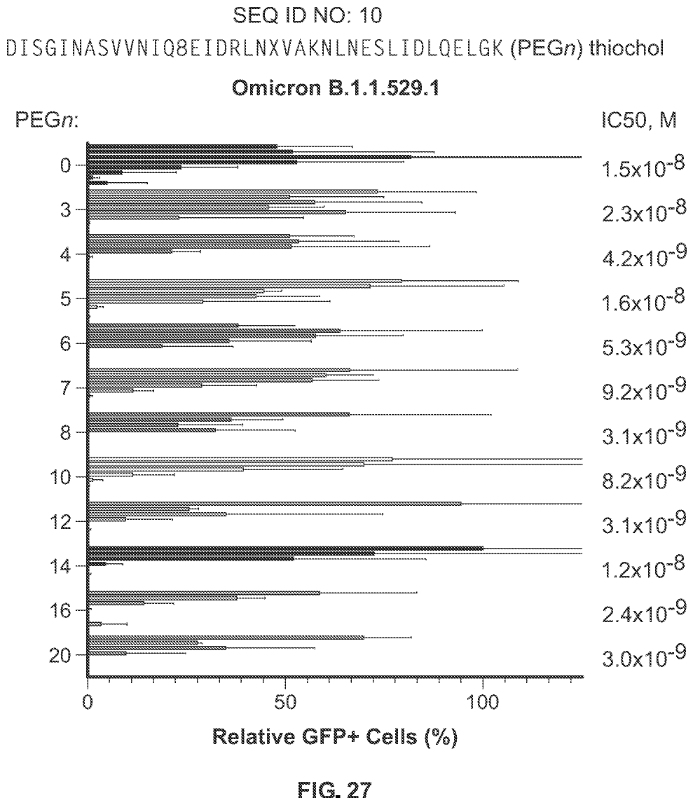

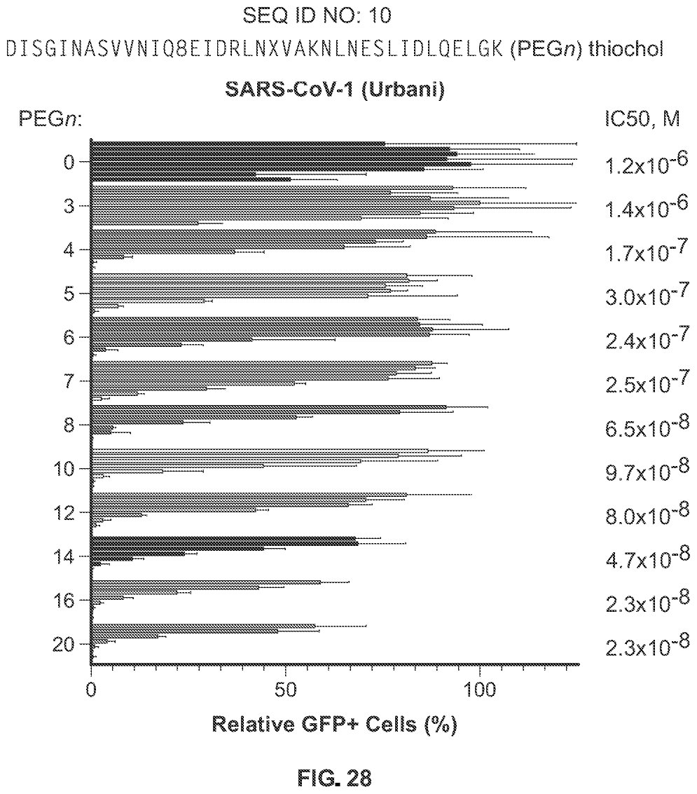

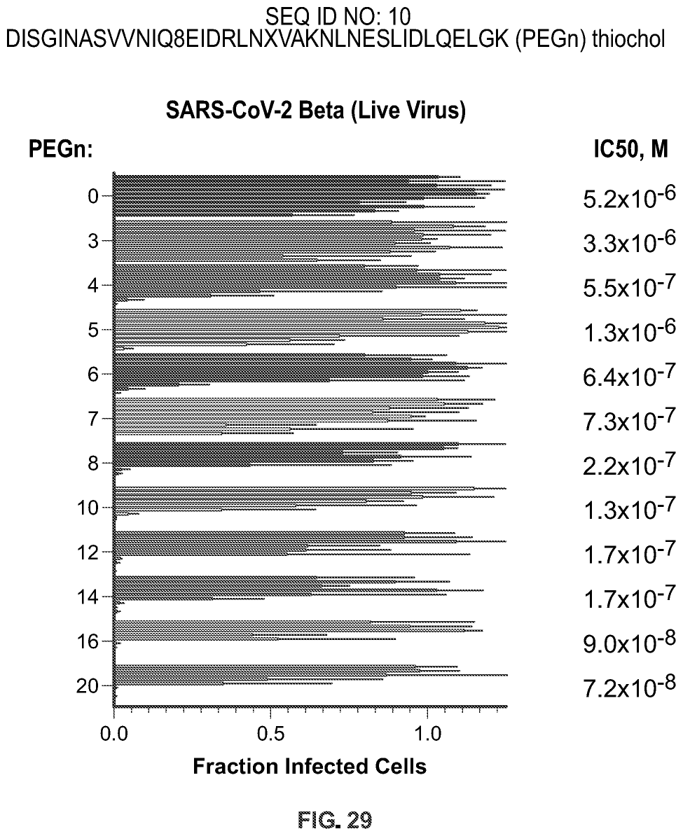

Next, we generated a series of i, i+7 stapled HR2 peptides of SEQ ID NO: 10 (Staple D) bearing even longer PEG(n) linkers, corresponding to n=10, 12, 14, 16, and 20. Across SARS-CoV-2 Omicron (FIG. 27) and SARS-CoV-1 (FIG. 28) pseudoviral assays, and live SARS-CoV-2 Beta strain (FIG. 29) viral assay, the longer linker lengths were as effective, and in some cases even more effective, than the PEG8 linker length. Taken together, we find that the length of the PEG linker between the stapled HR2 peptide and the thiocholesterol derivatization can be optimized to maximize antiviral activity, as evidenced across pseudovirus and live virus assays, with PEG8 and longer PEG lengths having the best activity, and compared to the series of other constructs, PEG0 and PEG3 showing the least potent activity. The superiority of the PEG8 and longer linkers, and the inferiority of PEG3 compared to the PEG4-8 and longer species, was unpredictable and required experimental determination in SARS-CoV-2 antiviral assays.

### Example 4: Specificity of the Anti-Viral Mechanism of Action of Stapled SARS-CoV-2 HR2 Peptides Bearing a C-Terminal PEG(n)-Thiocholesterol

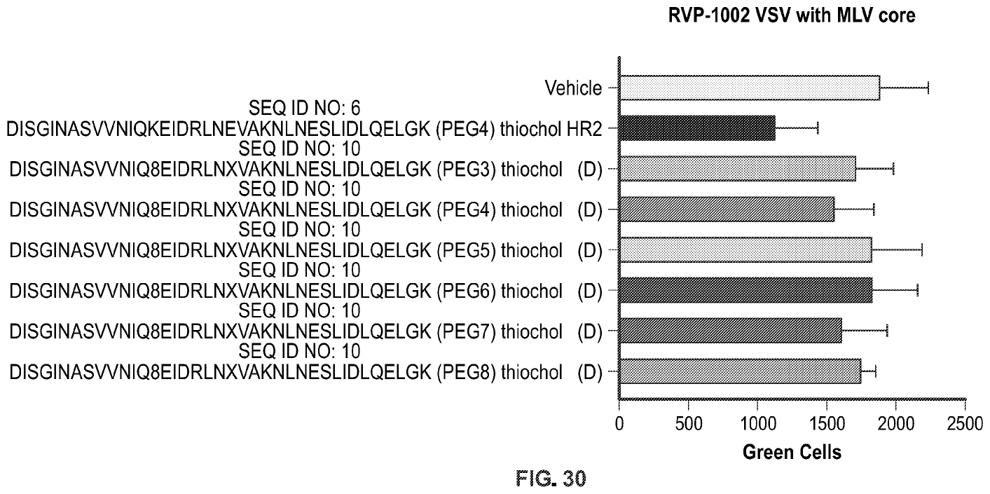

To examine the specificity of the peptides of the invention for SARS-CoV-2 virus, the series of i, i+7 stapled SARS-CoV-2 HR2 peptides bearing a PEG(n)-thiocholesterol moiety of variable PEG length (n=3-8) (Staple D, SEQ ID NO: 10) were tested in a pseudoviral assay of VSV with MLV core. Whereas the corresponding unstapled peptide demonstrated some non-specific anti-viral activity (2.5 M dosing, 48 hours), none of the stapled HR2 constructs bearing a PEG(n)-thiocholesterol with PEG linkers of n=3, 4, 5, 6, 7, or 8 showed any anti-VSV pseudoviral activity (see, FIG. 30).

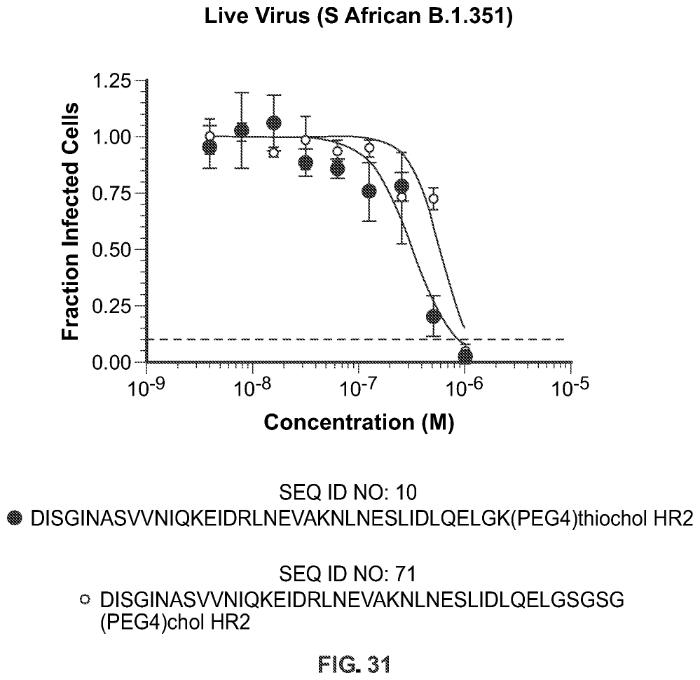

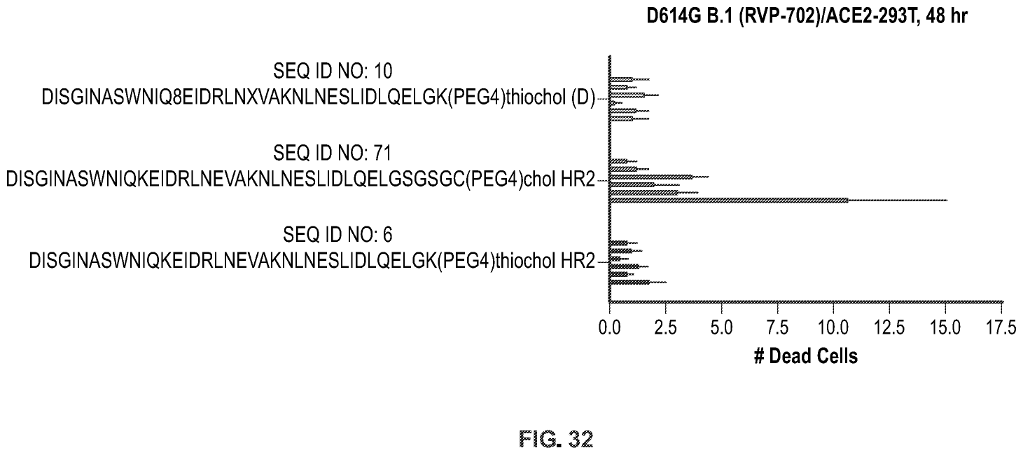

Next, we examined whether unstapled or the stapled SARS-CoV-2 HR2 peptides of the invention bearing a PEG(n)-thiocholesterol moiety generated synthetically on-resin exhibited any cytotoxicity in the antiviral dose-effective range. Comparing peptide of the invention SEQ ID NO:6 with an alternatively synthesized unstapled HR2 construct bearing a GSGSGC linker and PEG4-cholesterol appended at the C-terminus in solution (DeVries et al, Science, 2021), we find that the unstapled HR2 peptide of SEQ ID NO:6, and generated according to our above-described on-resin method, showed comparatively enhanced antiviral activity in a live virus assay (live virus: S. African B.1.351; cells: VeroB6; peptide dose-range 4-1000 nM) (see, FIG. 31). Further, the unstapled HR2 peptide of SEQ ID NO:6 and the corresponding i, i+7 stapled HR2 peptide of SEQ ID NO: 10 (Staple D, PEG4-thicholesterol) showed no cytotoxic activity across the anti-viral dose-effective range, as measured by DRAQ7 and Hoechst 33342 staining, whereas the unstapled HR2 peptide bearing a GSGSGC linker and C-terminally appended PEG4-cholesterol (DeVries et al, Science, 2021) killed cells within the dosing range (pseudovirus: D614G B.1; cells: 293T-ACE2; serial 2-fold dilution starting at 1 μM; read-out: 48 h) (see, FIG. 32).

### Example 5: SARS-CoV-2 5—HB Binding Activity and Broad SARS-CoV-2 Anti-Viral Activity Across Variants of a Stapled SARS-CoV-2 HR2 Peptide Bearing a C-Terminal PEG8-Chol

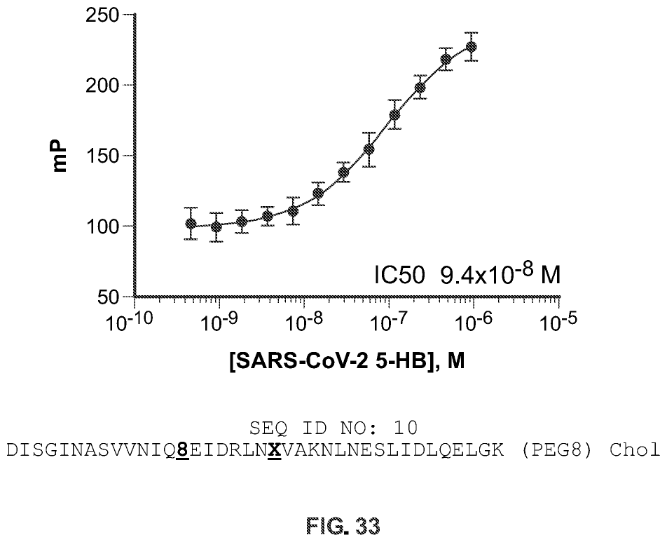

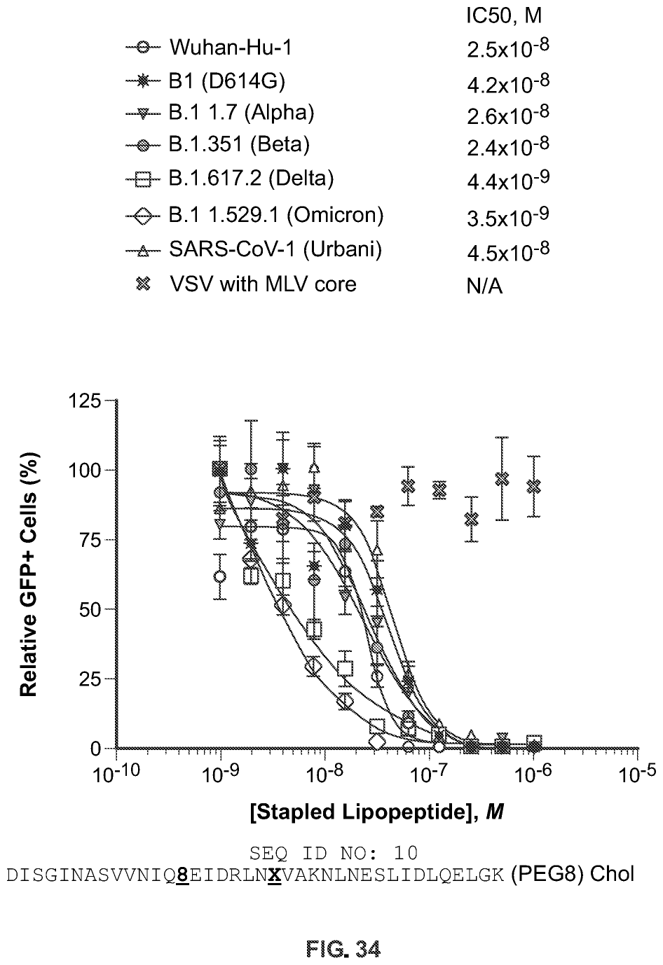

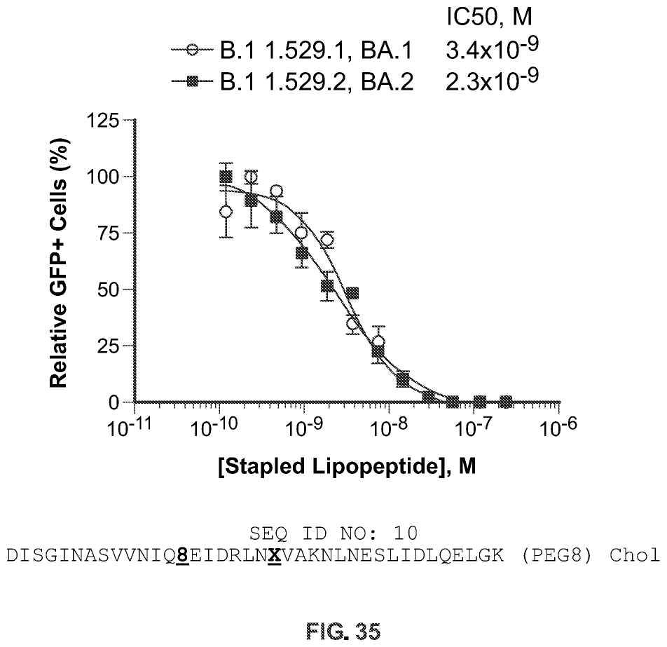

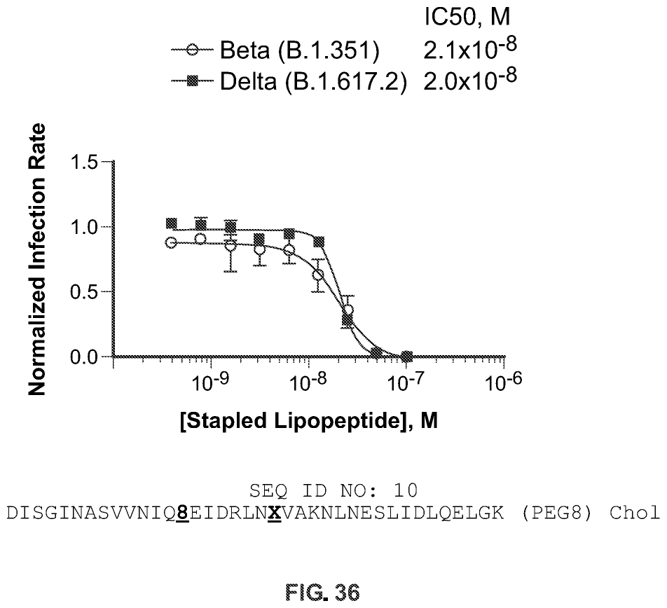

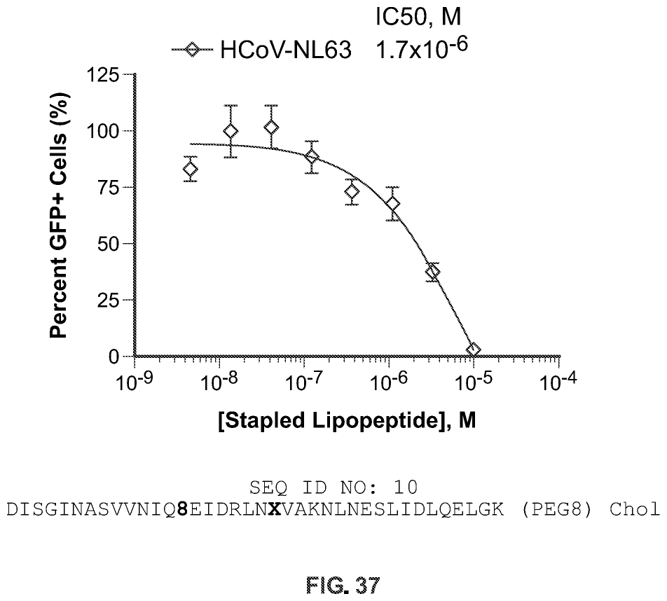

A direct fluorescence polarization binding assay revealed low nanomolar binding affinity of a stapled lipopeptide of composition corresponding to SEQ ID NO: 10 derivatized at the C-terminus with PEG8-Chol and at the N-terminus with FITC-β-Ala in place of the acetyl, to a recombinant five-helix bundle (5-HB) of SARS-CoV-2, with the addition of the FITC-stapled lipopeptide completing the fusogenic six helix bundle (peptide: 5 nM; 5-HB protein of sequence MQKLIANQFNSAIGKIQDSLSSTASALGKLQDVVNQNAQALNTLVKQLSSGGS GGDISGINASVVNIQKEIDRLNEVAKNLNESLIDLQELGSSGGQKLIANQFNSAI GKIQDSLSSTASALGKLQDVVNQNAQALNTLVKQLSSGGSGGDISGINASVVN IQKEIDRLNEVAKNLNESLIDLQELGSSGGQKLIANQFNSAIGKIQDSLSSTASA LGKLQDVVNQNAQALNTLVKQLSSSSGGHHHHHH (SEQ ID NO: 78) serial dilution from 1000 nM) (FIG. 33). The stapled lipopeptide of SEQ ID NO:10 derivatized at the C-terminus with PEG8-Chol demonstrated potent antiviral activity against (a) a spectrum of SARS-CoV-2 variant pseudoviruses (293-ACE2 cells, GFP-expressing pseudoviruses, peptide (serial dilution from 1000 nM), 48 hour read-out) (FIG. 34); (b) Omicron variants (293T-ACE2 cells, GFP-expressing pseudoviruses, peptide (serial dilution from 250 nM), 48 h read-out) (FIG. 35); (c) SARS-CoV-2 beta and delta live viruses (Vero cells, peptide (serial dilution from 100 nM), 48 hour read-out) (FIG. 36); and (d) common human coronaviruses, such as the alphacoronavirus NL63 (FIG. 37).

### Example 6: Preparation of a Stapled SARS-CoV-2 Peptide Derivatized with a C-Terminal PEG(8)-Cholesterol Moiety

The following compound was prepared based on procedures described in Example 1 above: a structurally stabilized peptide conjugate represented by the following formula:

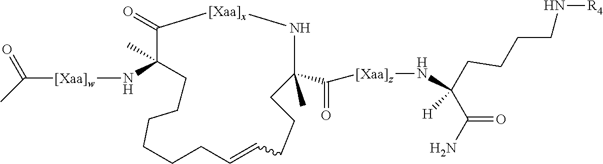

wherein
R₄ is —C(O)—(CH₂CH₂)—[O—CH₂CH_{2]8}—N(H)C(O)—(CH₂)—R₆;
R⁶ is

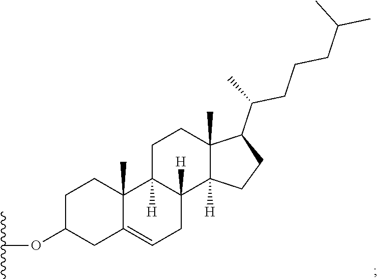

[Xaa]_{w} is DISGINASVVNIQ (SEQ ID NO: 35);
[Xaa]ₓ is EIDRLN (SEQ ID NO: 36); and
[Xaa]_{z} is VAKNLNESLIDLQELG (SEQ ID NO: 77).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A structurally-stabilized peptide comprising an amino acid sequence that is at least 85% identical to sequence set forth in SEQ ID NO: 6 (DISGINASVVNIQKEIDRLNEVAKNLNESLIDLQELGK), wherein amino acids at positions of SEQ ID NO:6 selected from (wherein position 1 is the N-terminal Aspartic Acid and position 38 is the C-terminal Lysine of SEQ ID NO: 6):
(i) positions 14 and 21,
(ii) positions 17 and 24,
(iii) positions 21 and 28, or
(iv) positions 24 and 31;
are replaced by α, α-disubstituted non-natural amino acids with olefinic side chains whose side chains are cross-linked;
wherein the structurally-stabilized peptide is 38 to 60 amino acids in length; and
wherein the structurally-stabilized peptide inhibits infection of a cell by SARS-CoV-2 in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

2.-14. (canceled)

15. A conjugate comprising the structurally-stabilized peptide of claim 1 and (i) polyethylene glycol (PEG) and/or (ii) cholesterol or thiocholesterol, wherein the (i) PEG and/or (ii) cholesterol or thiocholesterol are linked to the C-terminus of the structurally-stabilized peptide.

16.-28. (canceled)

29. A conjugate comprising:
a structurally-stabilized peptide of SEQ ID NO: 10 (DISGINASVVNIQ8EIDRLNXVAKNLNESLIDLQELGK), wherein 8 is a (R)-α-(7′-octenyl)alanine group or a (R)-α-(4′-pentenyl)alanine group, and X is a (S)-α-(4′-pentenyl)alanine group or a (S)-α-(7′-octenyl)alanine group; and
a PEG(n)-cholesterol or a PEG(n)-thiocholesterol moiety linked to the C-terminal lysine of the structurally-stabilized peptide, wherein n is 1-36.

30.-40. (canceled)

41. A structurally-stabilized peptide comprising the formula: 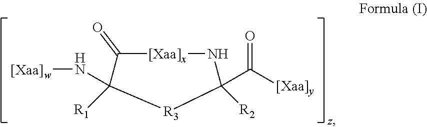
or a pharmaceutically acceptable salt thereof, wherein:
each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
z is 1;
(a) each [Xaa]_{w} is DISGINASVVNIQ (SEQ ID NO: 35), each [Xaa]ₓ is EIDRLN (SEQ ID NO: 36), and each [Xaa]_{y} is VAKNLNESLIDLQELGK (SEQ ID NO: 37);
(b) each [Xaa]_{w} is DISGINASVVNIQKEI (SEQ ID NO: 44), each [Xaa]ₓ is RLNEVA (SEQ ID NO: 45), and each [Xaa]_{y} is NLNESLIDLQELGK (SEQ ID NO: 46);
(c) each [Xaa]_{w} is DISGINASVVNIQKEIDRLN (SEQ ID NO: 56), each [Xaa]ₓ is VAKNLN (SEQ ID NO: 57), and each [Xaa]_{y} is SLIDLQELGK (SEQ ID NO: 58); or
(d) each [Xaa]_{w} is DISGINASVVNIQKEIDRLNEVA (SEQ ID NO: 65), each [Xaa]ₓ is NLNESL (SEQ ID NO: 66), and each [Xaa]_{y} is DLQELGK (SEQ ID NO: 67); and
wherein the structurally-stabilized peptide inhibits infection of a cell by SARS-CoV-2 in in pseudovirus and/or live SARS-CoV-2 virus assays and/or wherein the structurally-stabilized peptide prevents infection of a cell by SARS-CoV-2 in the pseudovirus and/or the live SARS-CoV-2 virus assays.

42.-49. (canceled)

50. A structurally stabilized peptide comprising Formula I-A, wherein Formula I-A is defined by: 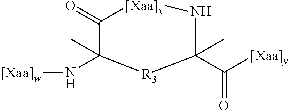
or a pharmaceutically acceptable salt thereof;
R₃ is alkenylene;
[Xaa]_{w} is
(SEQ ID NO: 35)
DISGINASVVNIQ;
[Xaa]ₓ is
(SEQ ID NO: 36)
EIDRLN;
and
[Xaa]_{y} is
(SEQ ID NO: 37)
VAKNLNESLIDLQELGK;
wherein optionally the amino group of the N-terminal aspartic acid in [Xaa]_{w} is replaced with —N(H)C(O)—(C₁₋₄ alkyl), and optionally the carboxylic acid group of the C-terminal lysine in [Xaa]_{y} is replaced with —C(O)NH₂.

51.-64. (canceled)

65. A conjugate comprising the structurally-stabilized peptide or pharmaceutically acceptable salt thereof of claim 41 and (i) PEG and/or (ii)cholesterol or thiocholesterol.

66. A conjugate comprising the structurally-stabilized peptide or pharmaceutically acceptable salt thereof of claim 50 and (i) PEG and/or (ii) cholesterol or thiocholesterol.

67.-121. (canceled)

122. A method of making a structurally-stabilized peptide, the method comprising: (a) providing a peptide having the sequence set forth in SEQ ID NO: 6 or a variant thereof, and (b) cross-linking the peptide.

123-124. (canceled)

125. A method of synthesizing a conjugate comprising a heptad repeat domain 2 (HR2) polypeptide, the method comprising (a) providing the HR2 polypeptide; and (b) derivatizing a resin bound amine of the HR2 polypeptide with (i) PEG and/or (ii) cholesterol or thiocholesterol containing a carboxylic acid on a resin.

126.-150. (canceled)

151. A linker comprising:
(a) a PEG(n)-thiocholesterol comprising the formula: 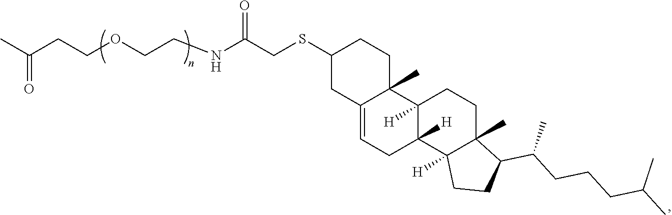
wherein n is 1-36; or
(b) a PEG(n)-cholesterol comprising the formula: 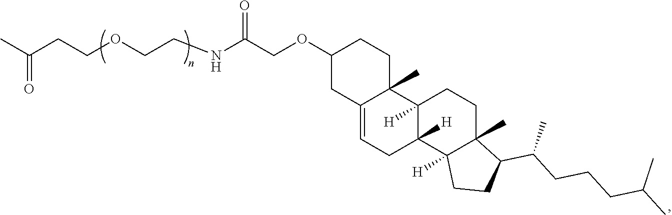
wherein n is 1-36.

152.-154. (canceled)

155. A pharmaceutical composition comprising the structurally-stabilized peptide of claim 1, and a pharmaceutically acceptable carrier.

156. A pharmaceutical composition comprising the structurally-stabilized peptide of claim 41, and a pharmaceutically acceptable carrier.

157. A pharmaceutical composition comprising the structurally-stabilized peptide of claim 50, and a pharmaceutically acceptable carrier.

158. A pharmaceutical composition comprising the conjugate of claim 15, and a pharmaceutically acceptable carrier.

159. A pharmaceutical composition comprising the conjugate of claim 29, and a pharmaceutically acceptable carrier.

160. A pharmaceutical composition comprising the conjugate of claim 65, and a pharmaceutically acceptable carrier.

161. A pharmaceutical composition comprising the conjugate of claim 66, and a pharmaceutically acceptable carrier.

162. A method of treating a coronavirus infection in a subject in need thereof, the method comprising administering to the subject a therapeutically-effective amount of the structurally-stabilized peptide of claim 1, wherein the subject is selected from a human, a cow, a pig, a horse, a cat, a dog, a rat, a mouse, or a bat.

163. A method of treating a coronavirus infection in a subject in need thereof, the method comprising administering to the subject a therapeutically-effective amount of the structurally-stabilized peptide of claim 41, wherein the subject is selected from a human, a cow, a pig, a horse, a cat, a dog, a rat, a mouse, or a bat.

164. A method of treating a coronavirus infection in a subject in need thereof, the method comprising administering to the subject a therapeutically-effective amount of the structurally-stabilized peptide of claim 50, wherein the subject is selected from a human, a cow, a pig, a horse, a cat, a dog, a rat, a mouse, or a bat.

165. A method of treating a coronavirus infection in a subject in need thereof, the method comprising administering to the subject a therapeutically-effective amount of the conjugate of claim 15, wherein the subject is selected from a human, a cow, a pig, a horse, a cat, a dog, a rat, a mouse, or a bat.

166. A method of treating a coronavirus infection in a subject in need thereof, the method comprising administering to the subject a therapeutically-effective amount of the conjugate of claim 29, wherein the subject is selected from a human, a cow, a pig, a horse, a cat, a dog, a rat, a mouse, or a bat.

167. A method of treating a coronavirus infection in a subject in need thereof, the method comprising administering to the subject a therapeutically-effective amount of the conjugate of claim 65, wherein the subject is selected from a human, a cow, a pig, a horse, a cat, a dog, a rat, a mouse, or a bat.

168. A method of treating a coronavirus infection in a subject in need thereof, the method comprising administering to the subject a therapeutically-effective amount of the conjugate of claim 66, wherein the subject is selected from a human, a cow, a pig, a horse, a cat, a dog, a rat, a mouse, or a bat.

169. A nanoparticle composition comprising the structurally-stabilized peptide of claim 1.

170. A nanoparticle composition comprising the structurally-stabilized peptide of claim 41.

171. A nanoparticle composition comprising the structurally-stabilized peptide of claim 50.

172. A nanoparticle composition comprising the conjugate of claim 15.

173. A nanoparticle composition comprising the conjugate of claim 29.

174. A nanoparticle composition comprising the conjugate of claim 65.

175. A nanoparticle composition comprising the conjugate of claim 66.


SEQUENCE LISTING (78 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 73 aa, Severe acute respiratory syndrome-related coronavirus
TQNVLYENQKLIANQFNSAIGKIQDSLSSTASALGKLQDVVNQNAQALNTLVKQLSSNFGAISSVLNDILSRL

SEQ ID NO: 3 - protein, 51 aa, Severe acute respiratory syndrome-related coronavirus
DVDLGDISGINASVVNIQKEIDRLNEVAKNLNESLIDLQELGKYBQYIKWP

SEQ ID NO: 4 - protein, 19 aa, Severe acute respiratory syndrome-related coronavirus
IQKEIDRLNEVAKNLNESL

SEQ ID NO: 5 - protein, 36 aa, synthetic construct
DISGINASVVNIQKEIDRLNEVAKNLNESLIDLQEL

SEQ ID NO: 6 - protein, 38 aa, synthetic construct
DISGINASVVNIQKEIDRLNEVAKNLNESLIDLQELGK

SEQ ID NO: 7 - protein, 37 aa, synthetic construct
  source 1..37: note = X = Any stapling amino acid
DISGINASVVIQKEIDXLNEVAKNLNESLIDLQELGK

SEQ ID NO: 8 - protein, 37 aa, synthetic construct
  source 1..37: note = X = Any stapling amino acid
DISGINASVVNQKEIDRXNEVAKNLNESLIDLQELGK

SEQ ID NO: 9 - protein, 37 aa, synthetic construct
  source 1..37: note = X =Any stapling amino acid
DISGINASVVNIKEIDRLXEVAKNLNESLIDLQELGK

SEQ ID NO: 10 - protein, 37 aa, synthetic construct
  source 1..37: note = X = Any stapling amino acid
DISGINASVVNIQEIDRLNXVAKNLNESLIDLQELGK

SEQ ID NO: 11 - protein, 37 aa, synthetic construct
  source 1..37: note = X = Any stapling amino acid
DISGINASVVNIQKIDRLNEXAKNLNESLIDLQELGK

SEQ ID NO: 12 - protein, 37 aa, synthetic construct
  source 1..37: note = X = Any stapling amino acid
DISGINASVVNIQKEDRLNEVXKNLNESLIDLQELGK

SEQ ID NO: 13 - protein, 37 aa, synthetic construct
  source 1..37: note = X = Any stapling amino acid
DISGINASVVNIQKEIRLNEVAXNLNESLIDLQELGK

SEQ ID NO: 14 - protein, 37 aa, synthetic construct
  source 1..37: note = X = Any stapling amino acid
DISGINASVVNIQKEIDLNEVAKXLNESLIDLQELGK

SEQ ID NO: 15 - protein, 37 aa, synthetic construct
  source 1..37: note = X = Any stapling amino acid
DISGINASVVNIQKEIDRNEVAKNXNESLIDLQELGK

SEQ ID NO: 16 - protein, 37 aa, synthetic construct
  source 1..37: note = X = Any stapling amino acid
DISGINASVVNIQKEIDRLEVAKNLXESLIDLQELGK

SEQ ID NO: 17 - protein, 37 aa, synthetic construct
  source 1..37: note = X = Any stapling amino acid
DISGINASVVNIQKEIDRLNVAKNLNXSLIDLQELGK

SEQ ID NO: 18 - protein, 37 aa, synthetic construct
  source 1..37: note = X = Any stapling amino acid
DISGINASVVNIQKEIDRLNEAKNLNEXLIDLQELGK

SEQ ID NO: 19 - protein, 37 aa, synthetic construct
  source 1..37: note = X = Any stapling amino acid
DISGINASVVNIQKEIDRLNEVKNLNESXIDLQELGK

SEQ ID NO: 20 - protein, 37 aa, synthetic construct
  source 1..37: note = X = Any stapling amino acid
DISGINASVVNIQKEIDRLNEVANLNESLXDLQELGK

SEQ ID NO: 21 - protein, 37 aa, synthetic construct
  source 1..37: note = X = Any stapling amino acid
DISGINASVVNIQKEIDRLNEVAKLNESLIXLQELGK

SEQ ID NO: 22 - protein, 319 aa, synthetic construct
MKDNTVPLTLISILADGEFHSGEQLGEQLGMSRAAINKHIKTLRDWGVDVFRVQGKGYCLPEPIQLLDEEKIRQQLDEGSVTVLPVIDSTNQYLLDRLDELTSGDVCIAEYQQAGRGSRGRKWFSPFGANLYLSMYWRLEQGPAAAMGLSLVIGIVMAETLQKLGADGVRVKWPNDLYLNDRKLAGILVEMTGKTGDAAHIVIGAGINLSMREPETDEVDQSWINLQEAGITIDRNQLAARLIKDLRSALRQFEQQGLAPFLSRWEALDNFINRPVKLIIGDREIHGIARGINEQGALLLEQDGVIKPWIGGEISLRSA

SEQ ID NO: 23 - protein, 319 aa, synthetic construct
MSEQWSRKREILALLSSGHFVSGEELATQLGISRTAVSKHIAALEEYGVDIYSVKGKGYKLANPISLIDESKLKSAINNRCFYFDEIPSTNGFMLKHAEELKSGDICVAEYQSAGRGRRGRTWVSPYGCHLYFSLYWRFPQGMAQAMGLSLVVACSLVKVLKSFGVDGVGVKWPNDIYLNHKKLAGVLIEMSGQADSECHLVIGIGINMAMSEQQGKKIDQPWSDLSSLTSMPDKTELLIALQKQLKQDLELFEREGLKAFQPRWQEADLFYGKQIKLLMGENQVEGICRGIDEQGAVLLETDDGIQAFIGGEISLRAA

SEQ ID NO: 24 - DNA, 957 bp, synthetic construct
  source 1..957: mol_type = other DNA
atgtctgagcagtggtctaggaagagagagatccttgctctcctcagctctggacatttcgtgtctggtgaggaacttgctactcagctcggaatctctagaaccgctgtgagcaaacatatcgctgcccttgaagagtacggcgtggacatctatagcgtgaaaggtaagggatacaagctcgcgaacccgatctctcttatcgacgagtctaagctcaagagcgccatcaacaaccgttgcttctacttcgacgagatcccgtctaccaacggcttcatgcttaaacacgctgaggaactcaagtccggggatatctgtgttgctgagtaccagtctgctggaagaggacgtagaggaagaacttgggtttcaccttacggatgccacctctacttcagcctttattggaggttcccgcaaggtatggctcaggctatgggactttctctcgttgttgcttgcagcctcgttaaggtgctcaagtctttcggagttgatggtgtgggagtgaagtggcctaacgacatctacctcaaccataagaaactcgccggtgtgctcatcgagatgtctggacaagctgattctgagtgccatctcgttatcggcatcgggatcaacatggctatgtctgaacagcaggggaagaagatcgatcagccttggtctgacctctcctctctcacttctatgcctgacaagaccgagctgcttatcgctctccaaaagcagcttaagcaggacctcgagcttttcgagagagagggacttaaggctttccagcctagatggcaagaggctgatctcttctacgggaagcagatcaagcttctcatgggagagaatcaggtcgagggaatctgcagaggaattgatgagcagggtgctgttctcctcgagactgatgatggaatccaggcttttatcggcggggagatctctttgagagctgct

SEQ ID NO: 25 - protein, 304 aa, synthetic construct
MRPFPLLRLLSDGEFHSGQALAEALGVSRASIWNALRNAEALGVDVHAVRGRGYRLSEPLDWLDEAIVARHLGEKASFFDLHVLDSVDSTNTALMERALQGAPHGTCVAAERQTAGRGRRGRAWHAVLGGSLTFSLLWRFNLGLGSLSGLSLAVGLAVVRALNKLGVHGARLKWPNDVLTDYRKLAGILIELQGDMLGPAAAVIGIGLNVRLSEAARNAVDQAVVDLHSLCGAPADRNTLLADLLRELAAMLTAFEQDGFAPLRAEWEAHHAYQDKAVRLLLPDGAGVQGVARGVDEDGALLLE

SEQ ID NO: 26 - protein, 10 aa, synthetic construct
DISGINASVV

SEQ ID NO: 27 - protein, 6 aa, synthetic construct
IQKEID

SEQ ID NO: 28 - protein, 20 aa, synthetic construct
LNEVAKNLNESLIDLQELGK

SEQ ID NO: 29 - protein, 11 aa, synthetic construct
DISGINASVVN

SEQ ID NO: 30 - protein, 6 aa, synthetic construct
QKEIDR

SEQ ID NO: 31 - protein, 19 aa, synthetic construct
NEVAKNLNESLIDLQELGK

SEQ ID NO: 32 - protein, 12 aa, synthetic construct
DISGINASVVNI

SEQ ID NO: 33 - protein, 6 aa, synthetic construct
KEIDRL

SEQ ID NO: 34 - protein, 18 aa, synthetic construct
EVAKNLNESLIDLQELGK

SEQ ID NO: 35 - protein, 13 aa, synthetic construct
DISGINASVVNIQ

SEQ ID NO: 36 - protein, 6 aa, synthetic construct
EIDRLN

SEQ ID NO: 37 - protein, 17 aa, synthetic construct
VAKNLNESLIDLQELGK

SEQ ID NO: 38 - protein, 14 aa, synthetic construct
DISGINASVVNIQK

SEQ ID NO: 39 - protein, 6 aa, synthetic construct
IDRLNE

SEQ ID NO: 40 - protein, 16 aa, synthetic construct
AKNLNESLIDLQELGK

SEQ ID NO: 41 - protein, 15 aa, synthetic construct
DISGINASVVNIQKE

SEQ ID NO: 42 - protein, 6 aa, synthetic construct
DRLNEV

SEQ ID NO: 43 - protein, 15 aa, synthetic construct
KNLNESLIDLQELGK

SEQ ID NO: 44 - protein, 16 aa, synthetic construct
DISGINASVVNIQKEI

SEQ ID NO: 45 - protein, 6 aa, synthetic construct
RLNEVA

SEQ ID NO: 46 - protein, 14 aa, synthetic construct
NLNESLIDLQELGK

SEQ ID NO: 47 - protein, 17 aa, synthetic construct
DISGINASVVNIQKEID

SEQ ID NO: 48 - protein, 6 aa, synthetic construct
LNEVAK

SEQ ID NO: 49 - protein, 13 aa, synthetic construct
LNESLIDLQELGK

SEQ ID NO: 50 - protein, 18 aa, synthetic construct
DISGINASVVNIQKEIDR

SEQ ID NO: 51 - protein, 6 aa, synthetic construct
NEVAKN

SEQ ID NO: 52 - protein, 12 aa, synthetic construct
NESLIDLQELGK

SEQ ID NO: 53 - protein, 19 aa, synthetic construct
DISGINASVVNIQKEIDRL

SEQ ID NO: 54 - protein, 6 aa, synthetic construct
EVAKNL

SEQ ID NO: 55 - protein, 11 aa, synthetic construct
ESLIDLQELGK

SEQ ID NO: 56 - protein, 20 aa, synthetic construct
DISGINASVVNIQKEIDRLN

SEQ ID NO: 57 - protein, 6 aa, synthetic construct
VAKNLN

SEQ ID NO: 58 - protein, 10 aa, synthetic construct
SLIDLQELGK

SEQ ID NO: 59 - protein, 21 aa, synthetic construct
DISGINASVVNIQKEIDRLNE

SEQ ID NO: 60 - protein, 6 aa, synthetic construct
AKNLNE

SEQ ID NO: 61 - protein, 9 aa, synthetic construct
LIDLQELGK

SEQ ID NO: 62 - protein, 22 aa, synthetic construct
DISGINASVVNIQKEIDRLNEV

SEQ ID NO: 63 - protein, 6 aa, synthetic construct
KNLNES

SEQ ID NO: 64 - protein, 8 aa, synthetic construct
IDLQELGK

SEQ ID NO: 65 - protein, 23 aa, synthetic construct
DISGINASVVNIQKEIDRLNEVA

SEQ ID NO: 66 - protein, 6 aa, synthetic construct
NLNESL

SEQ ID NO: 67 - protein, 7 aa, synthetic construct
DLQELGK

SEQ ID NO: 68 - protein, 24 aa, synthetic construct
DISGINASVVNIQKEIDRLNEVAK

SEQ ID NO: 69 - protein, 6 aa, synthetic construct
LNESLI

SEQ ID NO: 70 - protein, 6 aa, synthetic construct
LQELGK

SEQ ID NO: 71 - protein, 42 aa, synthetic construct
DISGINASVVNIQKEIDRLNEVAKNLNESLIDLQELGSGSGC

SEQ ID NO: 72-73 - skipped (2 entries)

SEQ ID NO: 74 - protein, 36 aa, synthetic construct
DISGINASVVNIQEIDRLNXVAKNLNESLIDLQELG

SEQ ID NO: 75 - protein, 13 aa, synthetic construct
DISGINASVVNIQ

SEQ ID NO: 76 - protein, 16 aa, synthetic construct
VAKNLNESLIDLQELG

SEQ ID NO: 77 - protein, 16 aa, synthetic construct
VAKNLNESLIDLQELG

SEQ ID NO: 78 - protein, 250 aa, synthetic construct
MQKLIANQFNSAIGKIQDSLSSTASALGKLQDVVNQNAQALNTLVKQLSSGGSGGDISGINASVVNIQKEIDRLNEVAKNLNESLIDLQELGSSGGQKLIANQFNSAIGKIQDSLSSTASALGKLQDVVNQNAQALNTLVKQLSSGGSGGDISGINASVVNIQKEIDRLNEVAKNLNESLIDLQELGSSGGQKLIANQFNSAIGKIQDSLSSTASALGKLQDVVNQNAQALNTLVKQLSSSSGGHHHHHH